SEQUENCE LISTING (13 sequences)

SEQ ID NO: 1 - protein, 3008 aa, Hepatitis C virus
MSTNPKPQRKTKRNTNRRPMDVKFPGGGQIVGGVYLLPRRGPRLGVRATRKTSERSQPRGRRQPIPKARRPEGRSWAQPGYPWPLYGNEGCGWAGWLLSPRGSRPSWGPNDPRRRSRNLGKVIDTLTCGFADLMGYIPLVGAPVGGVARALAHGVRALEDGINYATGNLPGCSFSIFLLALLSCLTVPASAVNYRNVSGIYHVTNDCPNSSIVYEADHHILHLPGCVPCVREGNQSRCWVALTPTVAAPYIGAPLESLRSHVDLMVGAATVCSGLYIGDLCGGLFLVGQMFSFRPRRHWTTQDCNCSIYTGHITGHRMAWDMMMNWSPTTTLVLAQVMRIPTTLVDLLSGGHWGVLVGVAYFSMQANWAKVILVLFLFAGVDAETHVSGAAVGRSTAGLANLFSSGSKQNLQLINSNGSWHINRTALNCNDSLNTGFLASLFYTHKFNSSGCSERLACCKSLDSYGQGWGPLGVANISGSSDDRPYCWHYAPRPCGIVPASSVCGPVYCFTPSPVVVGTTDHVGVPTYTWGENETDVFLLNSTRPPHGAWFGCVWMNSTGFTKTCGAPPCEVNTNNGTWHCPTDCFRKHPETTYAKCGSGPWITPRCLIDYPYRLWHFPCTANFSVFNIRTFVGGIEHRMQAACNWTRGEVCGLEHRDRVELSPLLLTTTAWQILPCSFTTLPALSTGLIHLHQNIVDVQYLYGVGSAVVSWALKWEYVVLAFLLLADARVSACLWMMFMVSQVEAALSNLININAASAAGAQGFWYAILFICIVWHVKGRFPAAAAYAACGLWPLFLLLLMLPERAYAYDQEVAGSLGGAIVVMLTILTLSPHYKLWLARGLWWIQYFIARTEAVLHVYIPSFNVRGPRDSVIVLAVLVCPHLVFDITKYLLAILGPLHILQASLLRIPYFVRAQALVKICSLLRGVVYGKYFQMVVLKAGALTGTYIYDHLTPMSDWAATGLRDLAVALEPVVFTPMEKKVIVWGADTAACGDIIRGLPVSARLGNEILLGPADTETSKGWRLLAPITAYAQQTRGLFSTIVTSLTGRDTNENCGEVQVLSTATQSFLGTAVNGVMWTVYHGAGAKTISGPKGPVNQMYTNVDQDLVGWPAPPGVRSLAPCTCGSADLYLVTRHADVIPVRRRGDTRGALLSPRPISTLKGSSGGPLLCPMGHAAGIFRAAVCTRGVAKAVDFVPVESLETTMRSPVFTDNSTPPAVPQTYQVAHLHAPTGSGKSTKVPAAYAAQGYKVLVLNPSVAATLGFGVYMSKAYGIDPNIRSGVRTITTGAPITYSTYGKFLADGGCSGGAYDIIICDECHSTDSTTILGIGTVLDQAETAGVRLTVLATATPPGSVTTPHSNIEEVALPTTGEIPFYGKAIPLELIKGGRHLIFCHSKKKCDELARQLTSLGLNAVAYYRGLDVSVIPTSGDVVVCATDALMTGFTGDFDSVIDCNTSVIQTVDFSLDPTFSIETTTVPQDAVSRSQRRGRTGRGRLGTYRYVTPGERPSGMFDTAVLCECYDAGCAWYELTPAETTTRLKAYFDTPGLPVCQDHLEFWESVFTGLTHIDGHFLSQTKQSGENFPYLVAYQATVCAKALAPPPSWDTMWKCLIRLKPTLHGPTPLLYRLGSVQNEVVLTHPITKYIMACMSADLEVVTSTWVLVGGVLAALAAYCLSVGSVVIVGRVVLSGQPAVIPDREVLYQQFDEMEECSKHLPLVEHGLQLAEQFKQKALGLLNFAGKQAQEATPVIQSNFAKLEQFWAKHMWNFISGIQYLAGLSTLPGNPAIASLMSFTAAVTSPLTTQQTLLFNILGGWVASQIATPTASTAFVVSGLAGAAVGSVGLGKILVDILAGYGAGVAGAVVTFKIMSGEMPSTEDLVNLLPAILSPGALVVGVVCAAILRRHVGPGEGAVQWMNRLIAFASRGNHVSPTHYVPESDAAARVTTILSSLTVTSLLRRLHKWINEDCSTPCAESWLWEVWDWVCTVLSDFKTWLKAKLLPLMPGIPFLSCQRGYKGEWRGDGVMHTTCPCGADLAGHIKNGSMRITGPKTCSNTWHGTFPINAYTTGPGVPIPAPNYKFALWRVSAEDYVEVRRVGDFHYVTGVTQDNIKCPCQVPAPEFFTEVDGIRLHRHAPKCKPLLRDEVSFSVGLNSFVVGSQLPCEPEPDVAVLTSMLTDPSHITAESARRRLARGSRPSLASSSASQLSAPSLKATCTAPHDSPGTDLLEANLLWGSTATRVETDEKVIILDSFESCVAEPNDDREVSVAAEILRPTKKFPPALPIWARPDYNPPLTETWKQQDYKPPTVHGCALPPGKQPPVPPPRRKRTVQLTESVVSTALAELAAKTFGQSEPSSDRDTDLTTPTETTDSGPIVVDDASDDGSYSSMPPLEGEPGDPDLTSDSWSTVSGSEDVVCCSMSYSWTGALVTPCAAEESKLPISPLSNSLLRHHNMVYATTTRSAVTRQKKVTFDRLQVVDSHYNEVLKEIKARASRVKARLLTTEEACDLTPPHSARSKFGYGAKDVRSHSRKAINHISSVWKDLLDDNNTPIPTTIMAKNEVFAVNPAKGGRKPARLIVYPDLGVRVCEKRALHDVIKKLPEAVMGAAYGFQYSPAQRVEFLLTAWKSKKTPMGFSYDTRCFDSTVTEKDIRVEEEVYQCCDLEPEARKVITALTDRLYVGGPMHNSKGDLCGYRRCRASGVYTTSFGNTLTCYLKATAAIRAAGLRDCTMLVCGDDLVVIAESDGVEEDNRALRAFTEAMTRYSAPPGDAPQPAYDLELITSCSSNVSVAHDVTGKKVYYLTRDPETPLARAAWETVRHTPVNSWLGNIIVYAPTIWVRMILMTHFFSILQSQEALEKALDFDMYGVTYSITPLDLPAIIQRLHGLSAFTLHGYSPHELNRVAGALRKLGVPPLRAWRHRARAVRAKLIAQGGRAKICGIYLFNWAVKTKLKLTPLPAAAKLDLSGWFTVGAGGGDIYHSMSHARPRYLLLCLLLLTVGVGIFLLPAR

SEQ ID NO: 2 - protein, 3008 aa, Hepatitis C virus
MSTNPKPQRKTKRNTNRRPMDVKFPGGGQIVGGVYLLPRRGPRLGVRATRKTSERSQPRGRRQPIPKARRPEGRSWAQPGYPWPLYGNEGCGWAGWLLSPRGSRPSWGPNDPRRRSRNLGKVIDTLTCGFADLMGYIPLVGAPVGGVARALAHGVRALEDGINYATGNLPGCSFSIFLLALLSCLTVPASAVNYRNVSGIYHVTNDCPNSSIVYEADHHILHLPGCVPCVREGNQSRCWVALTPTVAAPYIGAPLESLRSHVDLMVGAATGCSGLYIGDLCGGLFLVGQMFSFRPRRHWTTQDCNCSIYTGHITGHRMAWDMMMNWSPTTTLVLAQVMRIPTTLVDLLSGGHWGVLVGVAYFSMQANWAKVILVLFLFAGVDAETHVSGAAVGRSTAGLANLFSSGSKQNLQLINSNGSWHINRTALNCNDSLNTGFLASLFYTHKFNSSGCSERLARCKSLDSYGQGWGPLGVANISGSSDDRPYCWHYAPRPCGIVPASSVCGPVYCFTPSPVVVGTTDHVGVPTYTWGENETDVFLLNSTRPPHGAWFGCVWMNSTGFTKTCGAPPCEVNTNNGTWHCPTDCFRKHPETTYAKCGSGPWITPRCLIDYPYRLWHFPCTANFSVFNIRTFVGGIEHRMQAACNWTRGEVCGLEHRDRVELSPLLLTTTAWQILPCSFTTLPALSTGLIHLHQNIVDVQYLYGVGSAVVSWALKWEYVVLAFLLLADARVSACLWMMFMVSQVEAALSNLININAASAAGAQGFWYAILFICIVWHVKGRFPAAAAYAACGLWPLFLLLLMLPERAYAYDQEVAGSLGGAIVVMLAILTLSPHYKLWLARGLWWIQCFIARTEAVLHVYIPSFNVRGPRDSVIVLAVLVCPHLVFDITKYLLAILGPLHILQASLLRIPYFVRAQALVKICSLLRGVVYGKYFQMVVLKAGALTGTYIYDHLTPMSDWAATGLRDLAVALEPVVFTPMEKKVIVWGADTAACGDIIRGLPVSARLGNEILLGPADTETSKGWRLLAPITAYAQQTRGLFSTIVTSLTGRDTNENCGEVQVLSTATQSFLGTAVNGVMWTVYHGAGAKTISGPKGPVNQMYTNVDQDLVGWPAPPGVRSLAPCTCGSADLYLVTRHADVIPVRRRGDTRGALLSPRPISTLKGSSGGPLLCPMGHAAGIFRAAVCTRGVAKAVDFVPVESLETTMRSPVFTDNSTPPAVPQTYQVAHLHAPTGSGKSTKVPAAYAAQGYKVLVLNPSVAATLGFGVYMSKAYGIDPNIRSGVRTITTGAPVTYSTYGKFLADGGCSGGAYDIIICDECHSTDSTTILGIGTVLDQAETAGVRLTVLATATPPGSVTTPHSNIEEVALPTTGEIPFYGKAIPLELIKGGRHLIFCHSKKKCDELARQLTSLGLNAVAYYRGLDVSVIPTSGDVVVCATDALMTGFTGDFDSVIDCNTSVIQTVDFGMDPTFSIETTTVPQDAVSRSQRRGRTGRGRLGTYRYVTPGERPSGMFDTAVLCECYDAGCAWYELTPAETTTRLKAYFDTPGLPVCQDHLEFWESVFTGLTHIDGHLLSQTKQSGENFPYLVAYQATVCAKALAPPPSWDTMWKCLIRLKPTLHGPTPLLYRLGSVQNEVVLTHPITKYIMACMSADLEVVTSTWVLVGGVLAALASYCLSVGSVVIVGRVVLSGQPAVIPDREVLYQQFDEMEECSKHLPLVEHGLQLAEQFKQKALGLLNFAGKQAQEATPVIQSNFAKLEQFWAKHMWNFISGIQYLAGLSTLPGNPVIASLMSFTAAVTSPLTTQQTLLFNILGGWVASQIAAPTASTAFVVSGLAGAAVGSVGLGKILVDILAGYGAGVAGAVVAFKIMNGEMPSTEDLVNLLPAILSPGALVVGVVCAAILRRHVGPAEGAVQWMNRLIAFASRGNHVSPTHYVPESDAAARVTTILSSLTVTSLLRRLHKWINEDCSTPCTESWLWEVWDWVCTVLSDFKTWLKAKLLPLMPGIPFLSCQRGYKGEWRGDGVMHTTCPCGADLAGHIKNGSMRITGPKTCSNTWHGTFPINAYTTGPGVPIPAPNYKFALWRVSAEDYVEVRRVGDFHYVTGVTQDNIKCPCQVPAPEFFTEVDGIRLHRHAPKCKPLLRDEVSFSVGLNSFVVGSQLPCEPEPDVAVLTSMLTDPSHITAESARRRLARGSRPSLASSSASQLSAPSLKATCTAPHDSPGTDLLEANLLWGSTATRVETDEKVIILDSFESCVTEPNDDREVSVAAEILRPTKKFPPALPIWARPDYNPPLTETWKQQDYKPPTVHGCALPPGKQPPVPPPRRKRAVQLTESVVSTALAELAAKTFGQSEPSSDRDTDLTTPTETTDSGPIVVDDASDDGSYSSMPPLEGEPGDPDLTSDSWSTVSGSEGVVCCSMSYSWTGALVTPCAAEESKLPISPLSNSLLRHHNMVYATTTRSAVTRQKKVTFDRLQVVDSHYNEVLKEIKARASRVKARLLTTEEACDLTPPHSARSKFGYGAKDVRSHSRKAINHISSVWKDLLEDNNTPIPTTIMAKNEVFAVNPAKGGRKPARLIVYPDLGVRVCEKRALHDVINKLPEAVMGAAYGFQYSPAQRVEFLLTAWKSKKTPMGFSYDTRCFDSTVTEKDIRVEEEVYQCCDLEPEARKVITALTERLYVGGPMHNSKGDLCGYRRCRASGVYTTSFGNTLTCYLKATAAIRAAGLRDCTMLVCGDDLVVIAESDGVEEDNRALRAFTEAMTRYSAPPGDAPQPAYDLELITSCSSNVSVAHDATGKRVYYLTRDPTTPLARAAWETVRHTPVNSWLGNIIVYAPTIWVRMILMTHFFSILQSQEALEKALDFDMYGVTYSITPLDLPAIIQRLHGLSAFTLHGYSPHELNRVAGALRKLGVPPLRVWRHRARAVRAKLIAQGGRAKICGIYLFNWAVKTKLKLTPLPAAAKLDLSGWFTVGAGGGGIFHSVPHARPRYLLRYLLLLTVGVGIFLLPAR

SEQ ID NO: 3 - protein, 3008 aa, Hepatitis C virus; identical to SEQ ID NO: 2

SEQ ID NO: 4 - protein, 3008 aa, Hepatitis C virus
MSTNPKPQRKTKRNTNRRPMDVKFPGGGQIVGGVYLLPRRGPRLGVRATRKTSERSQPRGRRQPIPKARRPEGRSWAQPGYPWPLYGNEGCGWAGWLLSPRGSRPSWGPNDPRRRSRNLGKVIDTLTCGFADLMGYIPLVGAPVGGVARALAHGVRALEDGINYATGNLPGCSFSIFLLALLSCLTVPASAVNYRNVSGIYHVTNDCPNSSIVYEADHHILHLPGCVPCVREGNQSRCWVALTPTVAAPYIGAPLESLRSHVDLMVGAATGCSGLYIGDLCGGLFLVGQMFSFRPRRHWTTQDCNCSIYTGHITGHRMAWDMMMNWSPTTTLVLAQVMRIPTTLVDLLSGGHWGVLVGVAYFSMQANWAKVILVLFLFAGVDAETHVSGAAVGRSTAGLANLFSSGSKQNLQLINSNGSWHINRTALNCNDSLNTGFLASLFYTHKFNSSGCSERLARCKSLDSYGQGWGPLGVANISGSSDDRPYCWHYAPRPCGIVPASSVCGPVYCFTPSPVVVGTTDHVGVPTYTWGENETDVFLLNSTRPPHGAWFGCVWMNSTGFTKTCGAPPCEVNTNNGTWHCPTDCFRKHPETTYAKCGSGPWITPRCLIDYPYRLWHFPCTANFSVFNIRTFVGGIEHRMQAACNWTRGEVCGLEHRDRVELSPLLLTTTAWQILPCSFTTLPALSTGLIHLHQNIVDVQYLYGVGSAVVSWALKWEYVVLAFLLLADARVSACLWMMFMVSQVEAALSNLININAASAAGAQGFWYAILFICIVWHVKGRFPAAAAYAACGLWPLFLLLLMLPERAYAYDQEVAGSLGGAIVVMLAILTLSPHYKLWLARGLWWIQCFIARTEAVLHVYIPSFNVRGPRDSVIVLAVLVCPHLVFDITKYLLAILGPLHILQASLLRIPYFVRAQALVKICSLLRGVVYGKYFQMVVLKAGALTGTYIYDHLTPMSDWAATGLRDLAVALEPVVFTPMEKKVIVWGADTAACGDIIRGLPVSARLGNEILLGPADTETSKGWRLLAPITAYAQQTRGLFSTIVTSLTGRDTNENCGEVQVLSTATQSFLGTAVNGVMWTVYHGAGAKTISGPKGPVNQMYTNVDQDLVGWPAPPGVRSLAPCTCGSADLYLVTRHADVIPVRRRGDTRGALLSPRPISTLKGSSGGPLLCPMGHAAGIFRAAVCTRGVAKAVDFVPVESLETTMRSPVFTDNSTPPAVPQTYQVAHLHAPTGSGKSTKVPAAYAAQGYKVLVLNPSVAATLGFGVYMSKAYGIDPNIRSGVRTITTGAPVTYSTYGKFLADGGCSGGAYDIIICDECHSTDSTTILGIGTVLDQAETAGVRLTVLATATPPGSVTTPHSNIEEVALPTTGEIPFYGKAIPLELIKGGRHLIFCHSKKKCDELARQLTSLGLNAVAYYRGLDVSVIPTSGDVVVCATDALMTGFTGDFDSVIDCNTSVIQTVDFGMDPTFSIETTTVPQDAVSRSQRRGRTGRGRLGTYRYVTPGERPSGMFDTAVLCECYDAGCAWYELTPAETTTRLKAYFDTPGLPVCQDHLEFWESVFTGLTHIDGHLLSQTKQSGENFPYLVAYQATVCAKALAPPPSWDTMWKCLIRLKPTLHGPTPLLYRLGSVQNEVVLTHPITKYIMACMSADLEVVTSTWVLVGGVLAALASYCLSVGSVVIVGRVVLSGQPAVIPDREVLYQQFDEMEECSKHLPLVEHGLQLAEQFKQKALGLLNFAGKQAQEATPVIQSNFAKLEQFWAKHMWNFISGIQYLAGLSTLPGNPVIASLMSFTAAVTSPLTTQQTLLFNILGGWVASQIAAPTASTAFVVSGLAGAAVGSVGLGKILVDILAGYGAGVAGAVVAFKIMNGEMPSTEDLVNLLPAILSPGALVVGVVCAAILRRHVGPAEGAVQWMNRLIAFASRGNHVSPTHYVPESDAAARVTTILSSLTVTSLLRRLHKWINEDCSTPCAESWLWEVWDWVCTVLSDFKTWLKAKLLPLMPGIPFLSCQRGYKGEWRGDGVMHTTCPCGADLAGHIKNGSMRITGPKTCSNTWHGTFPINAYTTGPGVPIPAPNYKFALWRVSAEDYVEVRRVGDFHYVTGVTQDNIKCPCQVPAPEFFTEVDGIRLHRHAPKCKPLLRDEVSFSVGLNSFVVGSQLPCEPEPDVAVLTSMLTDPSHITAESARRRLARGSRPSLASSSASQLSAPSLKATCTAPHDSPGTDLLEANLLWGSTATRVETDEKVIILDSFESCVTEPNDDREVSVAAEILRPTKKFPPALPIWARPDYNPPLTETWKQQDYKPPTVHGCALPPGKQPPVPPPRRKRAVQLTESVVSTALAELAAKTFGQSEPSSDRDTDLTTPTETTDSGPIVVDDASDDGSYSSMPPLEGEPGDPDLTSDSWSTVSGSEGVVCCSMSYSWTGALVTPCAAEESKLPISPLSNSLLRHHNMVYATTTRSAVTRQKKVTFDRLQVVDSHYNEVLKEIKARASRVKARLLTTEEACDLTPPHSARSKFGYGAKDVRSHSRKAINHISSVWKDLLEDNNTPIPTTIMAKNEVFAVNPAKGGRKPARLIVYPDLGVRVCEKRALHDVINKLPEAVMGAAYGFQYSPAQRVEFLLTAWKSKKTPMGFSYDTRCFDSTVTEKDIRVEEEVYQCCDLEPEARKVITALTERLYVGGPMHNSKGDLCGYRRCRASGVYTTSFGNTLTCYLKATAAIRAAGLRDCTMLVCGDDLVVIAESDGVEEDNRALRAFTEAMTRYSAPPGDAPQPAYDLELITSCSSNVSVAHDATGKRVYYLTRDPTTPLARAAWETVRHTPVNSWLGNIIVYAPTIWVRMILMTHFFSILQSQEALEKALDFDMYGVTYSITPLDLPAIIQRLHGLSAFTLHGYSPHELNRVAGALRKLGVPPLRVWRHRARAVRAKLIARGGRAKICGIYLFNWAVKTKLKLTPLPAAAKLDLSGWFTVGAGGGGIFHSVPHARPRYLLRYLLLLTVGVGIFLLPAR

SEQ ID NO: 5 - protein, 3008 aa, Hepatitis C virus
MSTNPKPQRKTKRNTNRRPMDVKFPGGGQIVGGVYLLPRRGPRLGVRATRKTSERSQPRGRRQPIPKARRPEGRSWAQPGYPWPLYGNEGCGWAGWLLSPRGSRPSWGPNDPRRRSRNLGKVIDTLTCGFADLMGYIPLVGAPVGGVARALAHGVRALEDGINYATGNLPGCSFSIFLLALLSCLTVPASAVNYRNVSGIYHVTNDCPNSSIVYEADHHILHLPGCVPCVREGNQSRCWVALTPTVAAPYIGAPLESLRSHVDLMVGAATVCSGLYIGDLCGGLFLVGQMFSFRPRRHWTTQDCNCSIYTGHITGHRMAWDMMMNWSPTTTLVLAQVMRIPTTLVDLLSGGHWGVLVGVAYFSMQANWAKVILVLFLFAGVDAETHVSGAAVGRSTAGLANLFSSGSKQNLQLINSNGSWHINRTALNCNDSLNTGFLASLFYTHKFNSSGCSERLACCKSLDSYGQGWGPLGVANISGSSDDRPYCWHYAPRPCGIVPASSVCGPVYCFTPSPVVVGTTDHVGVPTYTWGENETDVFLLNSTRPPHGAWFGCVWMNSTGFTKTCGAPPCEVNTNNGTWHCPTDCFRKHPETTYAKCGSGPWITPRCLIDYPYRLWHFPCTANFSVFNIRTFVGGIEHRMQAACNWTRGEVCGLEHRDRVELSPLLLTTTAWQILPCSFTTLPALSTGLIHLHQNIVDVQYLYGVGSAVVSWALKWEYVVLAFLLLADARVSACLWMMFMVSQVEAALSNLININAASAAGAQGFWYAILFICIVWHVKGRFPAAAAYAACGLWPLFLLLLMLPERAYAYDQEVAGSLGGAIVVMLAILTLSPHYKLWLARGLWWIQYFIARTEAVLHVYIPSFNVRGPRDSVIVLAVLVCPHLVFDITKYLLAILGPLHILQASLLRIPYFVRAQALVKICSLLRGVVYGKYFQMVVLKAGALTGTYIYDHLTPMSDWAATGLRDLAVALEPVVFTPMEKKVIVWGADTAACGDIIRGLPVSARLGNEILLGPADTETSKGWRLLAPITAYAQQTRGLFSTIVTSLTGRDTNENCGEVQVLSTATQSFLGTAVNGVMWTVYHGAGAKTISGPKGPVNQMYTNVDQDLVGWPAPPGVRSLAPCTCGSADLYLVTRHADVIPVRRRGDTRGALLSPRPISTLKGSSGGPLLCPMGHAAGIFRAAVCTRGVAKAVDFVPVESLETTMRSPVFTDNSTPPAVPQTYQVAHLHAPTGSGKSTKVPAAYAAQGYKVLVLNPSVAATLGFGVYMSKAYGIDPNIRSGVRTITTGAPVTYSTYGKFLADGGCSGGAYDIIICDECHSTDSTTILGIGTVLDQAETAGVRLTVLATATPPGSVTTPHSNIEEVALPTTGEIPFYGKAIPLELIKGGRHLIFCHSKKKCDELARQLTSLGLNAVAYYRGLDVSVIPTSGDVVVCATDALMTGFTGDFDSVIDCNTSVIQTVDFGLDPTFSIETTTVPQDAVSRSQRRGRTGRGRLGTYRYVTPGERPSGMFDTAVLCECYDAGCAWYELTPAETTTRLKAYFDTPGLPVCQDHLEFWESVFTGLTHIDGHFLSQTKQSGENFPYLVAYQATVCAKALAPPPSWDTMWKCLIRLKPTLHGPTPLLYRLGSVQNEVVLTHPITKYIMACMSADLEVVTSTWVLVGGVLAALASYCLSVGSVVIVGRVVLSGQPAVIPDREVLYQQFDEMEECSKHLPLVEHGLQLAEQFKQKALGLLNFAGKQAQEATPVIQSNFAKLEQFWAKHMWNFISGIQYLAGLSTLPGNPVIASLMSFTAAVTSPLTTQQTLLFNILGGWVASQIAAPTASTAFVVSGLAGAAVGSVGLGKILVDILAGYGAGVAGAVVAFKIMNGEMPSTEDLVNLLPAILSPGALVVGVVCAAILRRHVGPGEGAVQWMNRLIAFASRGNHVSPTHYVPESDAAARVTTILSSLTVTSLLRRLHKWINEDCSTPCAESWLWEVWDWVCTVLSDFKTWLKAKLLPLMPGIPFLSCQRGYKGEWRGDGVMHTTCPCGADLAGHIKNGSMRITGPKTCSNTWHGTFPINAYTTGPGVPIPAPNYKFALWRVSAEDYVEVRRVGDFHYVTGVTQDNIKCPCQVPAPEFFTEVDGIRLHRHAPKCKPLLRDEVSFSVGLNSFVVGSQLPCEPEPDVAVLTSMLTDPSHITAESARRRLARGSRPSLASSSASQLSAPSLKATCTAPHDSPGTDLLEANLLWGSTATRVETDEKVIILDSFESCVAEPNDDREVSVAAEILRPTKKFPPALPIWARPDYNPPLTETWKQQDYKPPTVHGCALPPGKQPPVPPPRRKRTVQLTESVVSTALAELAAKTFGQSEPSSDRDTDLTTPTETTDSGPIVVDDASDDGSYSSMPPLEGEPGDPDLTSDSWSTVSGSEGVVCCSMSYSWTGALVTPCAAEESKLPISPLSNSLLRHHNMVYATTTRSAVTRQKKVTFDRLQVVDSHYNEVLKEIKARASRVKARLLTTEEACDLTPPHSARSKFGYGAKDVRSHSRKAINHISSVWKDLLEDNNTPIPTTIMAKNEVFAVNPAKGGRKPARLIVYPDLGVRVCEKRALHDVIKKLPEAVMGAAYGFQYSPAQRVEFLLTAWKSKKTPMGFSYDTRCFDSTVTEKDIRVEEEVYQCCDLEPEARKVITALTERLYVGGPMHNSKGDLCGYRRCRASGVYTTSFGNTLTCYLKATAAIRAAGLRDCTMLVCGDDLVVIAESDGVEEDNRALRAFTEAMTRYSAPPGDAPQPAYDLELITSCSSNVSVAHDVTGKRVYYLTRDPTTPLARAAWETVRHTPVNSWLGNIIVYAPTIWVRMILMTHFFSILQSQEALEKALDFDMYGVTYSITPLDLPAIIQRLHGLSAFTLHGYSPHELNRVAGALRKLGVPPLRVWRHRARAVRAKLIARGGRAKICGIYLFNWAVKTKLKLTPLPAAAKLDLSGWFTVGAGGGGIFHSVSHARPRYLLRYLLLLTVGVGIFLLPAR

SEQ ID NO: 6 - DNA, 9497 bp, Hepatits C virus
acctgctctctatgagagcaacactccaccatgaaccgctcccctgtgaggaactactgtcttcacgcagaaagcgtctagccatggcgttagtatgagtgttgtacagcctccaggaccccccctcccgggagagccatagtggtctgcggaaccggtgagtacaccggaatcgccgggatgaccgggtcctttcttggattaacccgctcaatgcccggaaatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgccgcccaatggacgttaagttcccgggtggtggccagatcgttggcggagtttacttgttgccgcgcaggggccccagattgggtgtgcgcgcgactcggaagacttcggagcggtcgcaacctcgtggaagacgccaacctatccccaaggcgcgtcgacccgagggaaggtcctgggcacaaccaggatatccatggcctctttacggtaatgagggttgtgggtgggcaggatggctcttgtccccccgtggctctcgaccgtcttggggcccaaatgatccccggcggaggtcccgcaatttgggtaaggtcatcgataccctaacctgcggcttcgccgacctcatgggatacatcccgctcgtaggcgcccccgtgggtggcgtcgccagggccctggcacatggtgtcagggctttggaggacgggatcaattatgcaacagggaatctccccggttgctccttttctatcttcctcttggcacttctttcgtgcctgactgtccccgcttcggccgttaactatcgcaatgtctcgggcatctaccatgtcaccaatgactgcccgaattcaagcatagtgtatgaggccgaccatcacatcttgcaccttccaggttgcgtgccctgcgtgagagaggggaatcagtcacgctgctgggtggcccttactcctaccgtcgcagcgccatacatcggcgcaccgcttgagtccttacggagtcatgtggatttgatggtgggggccgccactgtttgctcgggtctttacatcggggacctgtgtggcggcttgttcctagttggccagatgttttcattccgaccacggcgccactggaccacccaggattgcaattgttccatctacacagggcacattacaggccacagaatggcctgggacatgatgatgaactggagtccaacaaccaccttagttctcgcccaggtcatgaggatcccaaccactctggtagacttactctctggaggccactggggtgtcctcgtgggagtggcctatttcagcatgcaggccaattgggccaaagtcatcttggtcctattcctctttgcaggggttgatgccgagactcacgtgtctggggcagcagtcggccggagtaccgccggcttggctaacctcttttcttctgggtctaagcagaatttacagctcatcaacagcaatgggagctggcatataaataggactgcccttaactgcaatgacagcttaaacactgggttcttggctagcttgttctacacccacaagtttaacagctcagggtgttccgaacggctcgcgtgctgcaagagccttgacagctacggccaaggctggggcccactcggggtcgctaacatcagcggctcgtctgatgacaggccttattgctggcactacgcgcctcggccgtgcgggattgtgccagcatccagtgtgtgtggccccgtgtattgtttcactcccagccctgtcgtggtcggtactactgatcacgtcggggtccctacttacacctggggggagaatgagactgatgtcttccttttgaactcraccagaccgccgcatggtgcgtggtttggatgygtgtggatgaacagtaccgggttcaccaaaacctgtggcgcccctccatgcgaggttaacaccaataatgggacctggcactgccccaccgattgcttcaggaagcatccggagactacctacgccaagtgcggatcagggccttggatcacaccgcgctgcctgattgattacccgtaccggctgtggcatttcccgtgcaccgccaacttctccgtctttaacatcaggacatttgtcggcggtatagagcatcggatgcaagcggcatgcaactggaccaggggggaagtctgtggcttggagcacagggatcgcgtagagctatcacccctgctccttaccactacagcgtggcagatcctcccctgctctttcaccactttacctgccctctccaccggcttgatccacctccaccaaaatatcgtggacgtccagtacctctatggtgttggatctgcagtggtatcttgggcccttaagtgggaatatgtggtgctcgcgttcctgcttctcgcggacgcgagagtctctgcctgcctatggatgatgtttatggtaagtcaagttgaggcggctctgtccaacctaattaacatcaatgctgcttcagccgctggtgcccaaggcttctggtacgccatcctcttcatctgcattgtctggcatgtcaagggccggttcccagctgctgctgcctacgcagcctgcgggctgtggcccctgtttctcctgcttctgatgctgcctgagagggcttatgcatacgaccaggaagtggcagggtcccttggcggcgccatcgttgtcatgctgaccattctgacactgtctccgcactacaagttatggctggctaggggattgtggtggatccaatattttatagctaggaccgaggctgtgctgcatgtctatattccatccttcaacgtgcgcgggcctcgcgactcagtgattgttcttgcagtcctggtctgtccacacctagtatttgacatcacaaaatatcttctggccatcttagggcccctccacatactccaggcctcgctcctacgcatcccttactttgtgagggcacaagcgctggttaagatctgcagcttgttgcgtggggtagtttatggcaagtacttccaaatggtcgtgcttaaagcaggggccctgactggtacttacatctatgaccaccttactcccatgtcagattgggccgctacgggcctccgcgatttggcggtggccctagagccagttgtgttcacgcccatggagaagaaagtcatcgtctggggcgctgacaccgctgcgtgcggagacatcataaggggattacctgtttcggccaggttgggcaatgaaatcttgctcggaccagccgatacagaaacatcaaaggggtggagactccttgcccccatcacagcatacgcgcagcagacccgcggcttgttcagcaccatcgtaacgagcctcactggcagggacaccaatgagaattgtggcgaagtgcaggtcttatccaccgctacgcagtccttcctgggtactgcggttaacggcgtgatgtggaccgtctaccacggggcgggtgccaagaccatcagcggcccgaagggacctgtcaatcaaatgtacactaatgttgaccaagacttggtggggtggccagcaccccccggagtcagatctcttgctccgtgcacctgcggctcggcagacttgtatctagtcaccaggcacgcrgatgtaatacccgtgcgcaggagaggagacaccagaggagctctcttgagccctagaccaatatccactcttaagggatcttccggaggtccgctgctgtgccccatgggacacgccgcyggcatattccgtgcggcggtgtgtactcgaggggtagccaaggcggtagacttcgtcccggttgaatctcttgagactaccatgagatcaccagtgttcactgacaactcaacacccccagcagtgccccagacctaccaggtcgcgcacctacacgcaccaacaggaagtggcaagagcaccaaagtcccggcggcgtatgctgcccaaggctataaagtgctagtgctcaatccttcggttgcggccacactgggttttggggtatacatgtccaaggcatatggcatcgacccgaacatccggtcgggagtcaggaccatcaccacgggtgcgccaatcacgtactcaacgtatggtaagttcctggctgatggaggttgcagcggaggggcatacgacataatcatctgtgacgagtgccattccactgactccacaacgatccttggcataggcacagtcctggaccaagcggagaccgctggagtgcgcctcaccgtgctcgcgactgctactccgccagggtcagtgactacacctcattccaacatagaggaggtcgccctgccaacaacgggggaaatacccttttacggcaaggcgatccctctggagctgattaaggggggcagacatctcatcttctgccactcaaagaaaaagtgtgatgaactggccagacaactgacatctcttggtctgaatgccgtagcctactacagaggcttagacgtttcggtgattcccacgtctggggacgtcgtggtatgcgccacggacgccctcatgacgggttttaccggcgactttgactcagtgatagactgcaatacatctgtgatacagactgttgacttcagcttggaccccaccttctccatagagactacaaccgttccccaggacgcggtatcccgcagccagcggagaggccgcactggtagggggaggttgggcacataccggtatgtcaccccgggagagagaccatcaggcatgtttgacactgcagtgctttgcgagtgctacgatgccgggtgcgcctggtacgagctgacacctgctgaaaccacaacaaggctgaaagcttacttcgacacaccaggccttcctgtgtgccaagaccatctggagttctgggagagcgtctttacagggttaacccacatagacggtcatttcctatcccagaccaagcaatcgggtgagaatttcccgtatcttgttgcttaccaagccacggtgtgcgccaaggctctggcgcctccaccaagctgggacaccatgtggaagtgcctaattcgccttaagcccaccctgcacgggcccacacccctcctctacagactggggtctgtgcagaatgaagtggtgctcacccatcccatcaccaaatacatcatggcttgcatgtcagctgatctcgaggtagtgacaagtacgtgggtcttggtgggcggcgtcctggcagctctggctgcttactgtctttcagtgggcagcgtagtgattgttgggagagtcgtcctgtcgggccaacctgctgtcattcctgatcgcgaagtgctctaccaacagttcgacgaaatggaggagtgttccaaacacctcccactagtcgagcacgggttacaactggctgagcagttcaagcagaaggccttaggtctcctaaatttcgctggcaagcaagcccaagaggcaacaccagtgatccagtctaacttcgctaaacttgagcagttttgggcgaagcacatgtggaatttcatcagcggcattcaatatctcgctggactgtctaccttgccaggcaatcctgccattgcttccctcatgtcctttactgctgctgttacaagccctctgaccacccaacaaaccctcctttttaacatcttggggggatgggtggcctcgcagattgcgactccgacggcttctaccgcattcgtcgtgagcggcttggcgggggcggcagttggcagtgtgggccttggcaaaattttggtggacattctcgccggttacggcgccggcgtagctggcgctgtggttaccttcaagatcatgagcggcgagatgccttccacagaggacttggtaaatttgctcccggccattctatcgcccggagcattggtagtgggggtggtatgcgcggcgattttgcgccgccacgtgggcccgggcgaaggggctgtgcagtggatgaaccgtctaattgcgttcgcatcgcgaggcaatcacgtgtctcccacgcattacgtccctgagtccgacgcggcagcccgcgtgaccaccatactatcatccctcactgtgacatcccttctcagacgcctccacaagtggatcaatgaagattgctccaccccatgtgccgaatcttggctatgggaggtatgggattgggtctgcaccgtgctgagtgacttcaagacgtggctaaaagccaagttgctgcccctcatgccaggcatccccttcctctcatgccagaggggctataagggagagtggcgcggagatggcgtgatgcataccacatgcccctgcggagcagatctggcaggtcacatcaagaacggctcgatgagaatcaccgggccgaaaacctgcagcaacacatggcatggtaccttccccatcaatgcttacaccacaggccctggtgtacccatcccggcgccgaactacaagttcgcgctttggagggtgtccgccgaggactacgtggaggttcgcagagtgggtgatttccattatgtcaccggggtaacacaagacaacatcaagtgcccctgccaagttccggccccagagttcttcacggaagtggacggcatcaggctacaccgccacgccccgaagtgcaaacccttgctgcgggacgaagtgtcgttctcagtaggactcaattcgttcgtagtgggatcacaactcccatgcgagccagagccggacgtggcagtgctaacatccatgctgacagacccatcacacataacggcggaatcggcgcgtcggagattggctcgagggtcacgaccctcgctagctagttcctcggcgagtcagctttccgccccgtctctcaaggccacgtgtaccgctccccatgactcccctggtactgatctcctcgaggctaacctcttgtgggggtctaccgctaccagggttgagacggacgagaaggtaataatactagactcttttgagtcatgtgtggctgagccaaatgatgacagggaagtctcggttgccgcggaaatcctgcgtccgaccaagaagttccctccagcactaccgatctgggcccggccggattacaatccacctcttaccgagacgtggaagcagcaggactacaagcctccgaccgtccacgggtgcgctctgcctcccggcaagcagccccccgttcctcctcccaggaggaaacggacggtacagctcactgagtccgttgtttctaccgctttggcagagctggccgcaaagacctttggccagtcagagccgagctcagaccgtgatacagaccttaccaccccaactgagaccacagactcgggccccatcgtcgtggatgatgcatccgatgacggatcttattcgtcaatgcctccactagagggggagcccggtgacccggacttgacatcagactcttggtccactgttagcggatcggaggacgtcgtgtgctgctcaatgtcatattcatggactggggcgcttgtaacaccttgcgcggctgaagaatcaaagctgccaattagccccctgagcaattcacttttgcgccatcacaatatggtgtatgccacgaccacccgttctgctgtgacacggcagaagaaggtgaccttcgaccgcctgcaggtggtggacagtcactacaatgaagtgcttaaggagataaaggcacgagcatccagagtgaaggcacgcttgcttaccacagaggaagcttgcgacctgacgcccccccactcagccagatcaaagttcggctacggggcgaaggatgttcggagccattcccgcaaggccattaaccacatcagctccgtgtggaaggacttgctggacgacaacaataccccaataccaacaacaatcatggccaaaaatgaggtcttcgctgtgaacccagcgaagggaggtcggaagcctgctcgcctgatcgtgtatccggatctcggggtccgggtttgcgagaagagagcgcttcacgacgtcatcaaaaaactgcctgaggccgtgatgggagccgcttatggcttccaatactccccagcgcagcgggtggaatttcttctgactgcttggaagtcgaagaagaccccaatggggttctcttatgatacccgctgctttgactccactgtaaccgaaaaggacatcagggtcgaggaagaggtctatcagtgttgtgacctggagcccgaagcccgcaaagtcatcaccgccctcacagatagactctatgtgggcggccctatgcacaacagcaagggagacctttgtgggtatcggagatgtcgcgcaagcggcgtctacaccaccagcttcgggaacacgctgacgtgctatctcaaagccacggccgccatcagggcggcggggctgagagactgcactatgttggtttgcggtgatgacttagtcgtcatcgctgagagcgacggcgtagaggaggacaaccgagccctccgagccttcacggaggctatgacgagatactcggctcccccaggtgacgccccgcagccagcatatgacctggaactaataacatcatgttcatccaacgtctcagtcgcgcacgacgtgacgggtaaaaaggtatattacctaacccgagaccctgaaactcccttggcgcgagccgcatgggagacagtccgacacactccagtcaattcctggttgggaaacatcatagtctacgctcccacaatatgggtgcgcatgatattgatgacccactttttctcaatactccagagccaggaagcccttgagaaagcactcgacttcgatatgtacggagtcacctactctatcactccgctggatttaccggcaatcattcaaagactccatggcttaagcgcgttcacgctgcacggatactctccacacgaactcaaccgggtggccggagccctcagaaaacttggggtacccccgctgagagcgtggagacatcgggcccgagcagtccgcgctaagcttatcgcccagggaggtagagccaaaatatgtggcatatacctctttaactgggcggtaaaaaccaaactcaaactcactccattgcctgccgctgccaaactcgatttatcgggttggtttacggtaggcgccggcgggggagacatttatcacagcatgtctcatgcccgaccccgctatttactcctgtgcctactcctacttacagtaggggtaggcatcttcctgctgcctgctcggtaggcagcttaacactccgaccttagggtccccttgttttttttttttttttttttttttttttttttttttttttttttttcctttccttctttcctttcctaatctttctttcttggtggctccatcttag

SEQ ID NO: 7 - DNA, 9577 bp, Hepatitis C virus
acctgctctctatgagagcaacactccaccatgaaccactcccctgtgaggaactactgtcttcacgcagaaagcgtctagccatggcgttagtatgagtgttgtacagcctccaggaccccccctcccgggagagccatagtggtctgcggaaccggtgagtacaccggaatcgccgggatgaccgggtcctttcttggattaacccgctcaatgcccggaaatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgccgcccaatggacgttaagttcccgggtggtggccagatcgttggcggagtttacttgttgccgcgcaggggccccagattgggtgtgcgcgcgactcggaagacttcggagcggtcgcaacctcgtggaagacgccaacctatccccaaggcgcgtcgacccgagggaaggtcctgggcacaaccaggatatccatggcctctttacggtaatgagggttgtgggtgggcaggatggctcttgtccccccgtggctctcgaccgtcttggggcccaaatgatccccggcggaggtcccgcaatttgggtaaggtcatcgataccctaacctgcggcttcgccgacctcatgggatacatcccgctcgtaggcgcccccgtgggtggcgtcgccagggccctggcacatggtgtcagggctttggaggacgggatcaattatgcaacagggaatctccccggttgctccttttctatcttcctcttggcacttctttcgtgcctgactgtccccgcttcggccgttaactatcgcaatgtctcgggcatctaccatgtcaccaatgactgcccgaattcaagcatagtgtatgaggccgaccatcacatcttgcaccttccaggttgcgtgccctgcgtgagagaggggaatcagtcacgctgctgggtggcccttactcctaccgtcgcagcgccatacatcggcgcaccgcttgagtccttacggagtcatgtggatttgatggtgggggccgccactggttgctcgggtctttacatcggggacctgtgtggcggcttgttcctagttggccagatgttttcattccgaccacggcgccactggaccacccaggattgcaattgttccatctacacagggcacattacaggccacagaatggcctgggacatgatgatgaactggagtccaacaaccaccttagttctcgcccaggtcatgaggatcccaaccactctggtagacttactctctggaggccactggggtgtcctcgtgggagtggcctatttcagcatgcaggccaattgggccaaagtcatcttggtcctattcctctttgcaggggttgatgccgagactcacgtgtctggggcagcagtcggccggagtaccgccggcttggctaacctcttttcttctgggtctaagcagaatttacagctcatcaacagcaatgggagctggcatataaataggactgcccttaactgcaatgacagcttaaacactgggttcttggctagcttgttctacacccacaagtttaacagctcagggtgttccgaacggctcgcgcgctgcaagagccttgacagctacggccaaggctggggcccactcggggtcgctaacatcagcggctcgtctgatgacaggccttattgctggcactacgcgcctcggccgtgcgggattgtgccagcatccagtgtgtgtggccccgtgtattgtttcactcccagccctgtcgtggtcggtactactgatcacgtcggggtccctacttacacctggggggagaatgagactgatgtcttccttttgaactcgaccagaccgccgcatggtgcgtggtttggatgcgtgtggatgaacagtaccgggttcaccaaaacctgtggcgcccctccatgcgaggttaacaccaataatgggacctggcactgccccaccgattgcttcaggaagcatccggagactacctacgccaagtgcggatcagggccttggatcacaccgcgctgcctgattgattacccgtaccggctgtggcatttcccgtgcaccgccaacttctccgtctttaacatcaggacatttgtcggcggtatagagcatcggatgcaagcggcatgcaactggaccaggggggaagtctgtggcttggagcacagggatcgcgtagagctatcacccctgctccttaccactacagcgtggcagatcctcccctgctctttcaccactttacctgccctctccaccggcttgatccacctccaccaaaatatcgtggacgtccagtacctctatggtgttgggtctgcagtggtatcttgggcccttaagtgggaatatgtggtgctcgcgttcctgcttctcgcggacgcgagagtctctgcctgcctatggatgatgtttatggtaagtcaagttgaggcggctctgtccaacctgattaacatcaatgctgcttcagccgctggtgcccaaggcttctggtacgccatcctcttcatctgcattgtctggcatgtcaagggccggttcccagctgctgctgcctacgcagcctgcgggctgtggcccctgtttctcctgcttctgatgctgcctgagagggcttatgcatacgaccaggaagtggcagggtcccttggcggcgccatcgttgtcatgctggccattctgacactgtctccgcactacaagttatggctggctaggggattgtggtggatccaatgttttatagctaggaccgaggctgtgctgcatgtctatattccatccttcaacgtgcgcgggcctcgcgactcagtgattgttcttgcagtcctggtctgtccacacctagtatttgacatcacaaaatatcttctggccatcttagggcccctccacatactccaggcctcgctcctacgcatcccttactttgtgagggcacaagcgctggttaagatctgcagcttgttgcgtggggtagtttatggcaagtacttccaaatggtcgtgcttaaagcaggggccctgactggtacttacatctatgaccaccttactcccatgtcagattgggccgctacgggcctccgcgatttggcggtggccctagagccagttgtgttcacgcccatggagaagaaagtcatcgtctggggcgctgacaccgctgcgtgcggagacatcataaggggattacctgtttcggccaggttgggcaatgaaatcttgctcggaccagccgatacagaaacatcaaaggggtggagactccttgcccccatcacagcatacgcgcagcagacccgcggcttgttcagcaccatcgtaacgagcctcactggcagggacaccaatgagaattgtggcgaagtgcaggtcttatccaccgctacgcagtccttcctgggtactgcggttaacggcgtgatgtggaccgtctaccacggggcgggtgccaagaccatcagcggcccgaagggacctgtcaatcaaatgtacactaatgttgaccaagacttggtggggtggccagcaccccccggagtcagatctcttgctccgtgcacctgcggctcggcagacttgtatctagtcaccaggcacgcagatgtaatacccgtgcgcaggagaggagacaccagaggagctctcttgagccctagaccaatatccactcttaagggatcttccggaggtccgctgctgtgccccatgggacacgccgccggcatattccgtgcggcggtgtgtactcgaggggtagccaaggcggtagacttcgtcccggttgaatctcttgagactaccatgagatcaccagtgttcactgacaactcaacacctccagcagtgccccagacctaccaggtcgcgcacctacacgcaccaacaggaagtggcaagagcaccaaagtcccggcggcgtatgctgcccaaggctataaagtgctagtgctcaatccttcggttgcggccacactgggttttggggtatacatgtccaaggcatatggcatcgacccgaacatccggtcgggagtcaggaccatcaccacgggtgcgccagtcacgtactcaacgtatggtaagttcctggctgatggaggttgcagcggaggggcatacgacataatcatctgtgacgagtgccattccactgactccacaacgatccttggcataggcacagtcctggaccaagcggagaccgctggagtgcgcctcaccgtgctcgcgactgctactccgccagggtcagtgactacacctcattccaacatagaggaggtcgccctgccaacaacgggggaaatccccttttacggcaaggcgatccctctggagctgattaaggggggcagacatctcatcttctgccactcaaagaaaaagtgtgatgaactggccagacaactgacatctcttggtctgaatgccgtagcctactacagaggcttagacgtttcggtgattcccacgtctggggacgtcgtggtatgcgccacggacgccctcatgacgggttttaccggcgactttgactcagtgatagactgcaatacatctgtgatacagactgttgacttcggcatggaccccaccttctccatagagactacaaccgttccccaggacgcggtatcccgcagccagcggagaggccgcactggtagggggaggttgggcacataccggtatgtcaccccgggagagagaccatcaggcatgtttgacactgcagtgctttgcgagtgctacgatgccgggtgtgcctggtacgagctgacacctgctgaaaccacaacaaggctgaaagcttacttcgacacaccaggccttcctgtgtgccaagaccatctggagttctgggagagcgtctttacagggttaacccacatagacggtcatctcctatcccagaccaagcaatcgggtgagaatttcccgtatcttgttgcttaccaagccacggtgtgcgccaaggctctggcgcctccaccaagctgggacaccatgtggaagtgcctaattcgccttaagcccaccctgcacgggcccacacccctcctctacagactggggtctgtgcagaatgaagtggtgctcacccatcccatcaccaaatacatcatggcttgcatgtcagctgatctcgaggtagtgacaagtacgtgggtcttggtgggcggcgtcctggcagctctggcttcttactgtctttcagtgggcagcgtagtgattgttgggagagtcgtcctgtcgggccaacctgctgtcattcccgatcgcgaagtgctctaccaacagttcgacgaaatggaggagtgttccaaacacctcccactagtcgagcacgggttacaactggctgagcagttcaagcagaaggccttaggtctcctaaatttcgctggcaagcaagcccaagaggcaacaccagtgatccagtctaacttcgctaaacttgagcagttttgggcgaagcacatgtggaatttcatcagcggcattcaatatctcgctggactgtctaccttgccaggcaatcctgtcattgcttccctcatgtcctttactgctgctgttacaagccctctgaccacccaacaaaccctcctttttaacatcttggggggatgggtggcctcgcagattgcggctccgacggcttctaccgcattcgtcgtgagcggcttggcgggggcggcagttggcagtgtgggccttggcaaaattttggtggacattctcgccggttacggcgccggcgtagctggcgctgtggttgccttcaagatcatgaacggcgagatgccttccacagaggacttggtaaatttgctcccggccattctatcgcccggagcattggtagtgggggtggtatgcgcggcgattttgcgccgccacgtgggcccggccgaaggggctgtgcagtggatgaaccgtctaattgcgttcgcatcgcgaggcaatcacgtgtctcccacgcattacgtccctgagtccgacgcggcagcccgcgtgaccaccatactatcatccctcactgtgacatcccttctcagacgcctccacaagtggatcaatgaagattgctccaccccatgtaccgaatcttggctatgggaggtatgggattgggtctgcaccgtgctgagtgacttcaagacgtggctaaaagccaagttgctgcccctcatgccaggcatccccttcctctcatgccagaggggctataagggagagtggcgcggagatggcgtgatgcataccacatgcccctgcggagcagatctggcaggtcacatcaagaacggctcgatgagaatcaccgggccgaaaacctgcagcaacacatggcatggtaccttccccatcaatgcttacaccacaggccctggtgtacccatcccggcgccgaactacaagttcgcgctttggagggtgtccgccgaggactacgtggaggttcgcagagtgggtgatttccattatgtcaccggggtaacacaagacaacatcaagtgcccctgccaagttccggccccagagttcttcacggaagtggacggcatcaggctacaccgccacgccccgaagtgcaaacccttgctgcgggacgaagtgtcgttctcagtaggactcaattcgttcgtagtgggatcacaactcccatgcgagccagagccggacgtggcagtgctaacatccatgctgacagacccatcacacataacggcggaatcggcgcgtcggagattggctcgagggtcacgaccctcgctagctagttcctcggcgagtcagctttccgccccgtctctcaaggccacgtgtaccgctccccatgactcccctggtactgatctcctcgaggctaacctcttgtgggggtctaccgctaccagggttgagacggacgagaaggtaataatactagactcttttgagtcatgtgtgactgagccaaatgatgacagggaagtctcggttgccgcggaaatcctgcgtccgaccaagaagttccctccagcactaccgatctgggcccggccggattacaatccacctcttaccgagacgtggaagcagcaggactacaagcctccgaccgtccacgggtgcgctctgcctcccggcaagcagccccccgttcctcctcccaggaggaaacgggcggtacagctcactgagtccgttgtttctaccgctttggcagagctggccgcaaagacctttggccagtcagagccgagctcagaccgtgatacagaccttaccaccccaactgagaccacagactcgggccccatcgtcgtggatgatgcatccgatgacggatcttattcgtcaatgcctccactagagggggagcccggtgacccggacttgacatcagactcttggtccactgttagcggatcggagggcgtcgtgtgctgctcaatgtcatattcatggactggggcgcttgtaacaccttgcgcggctgaagaatcaaagctgccaattagccccctgagcaattcacttttgcgccatcacaatatggtgtatgccacgaccacccgttctgctgtgacacggcagaagaaggtgaccttcgaccgcctgcaggtggtggacagtcactacaatgaagtgcttaaggagataaaggcacgagcatccagagtgaaggcacgcttgcttaccacagaggaagcttgcgacctgacgcccccccactcagccagatcaaagttcggctacggggcgaaggatgttcggagccattcccgcaaggccattaaccacatcagctccgtgtggaaggacttgctggaagacaacaataccccaataccaacaacaatcatggccaaaaatgaggtcttcgctgtgaacccagcgaagggaggtcggaagcctgctcgcctgatcgtgtatccggatctcggggtccgggtttgcgagaagagagcgcttcacgacgtcatcaataaactgcctgaggccgtgatgggagccgcttatggcttccaatactccccagcgcagcgggtggaatttcttctgactgcttggaagtcgaagaagaccccaatggggttctcttatgatacccgctgctttgactccactgtaaccgaaaaggacatcagggtcgaggaagaggtctatcagtgttgtgacctggagcccgaagcccgcaaagtcatcaccgccctcacagagagactctatgtgggcggccctatgcacaacagcaagggagacctttgtgggtatcggagatgtcgcgcaagcggcgtctacaccaccagcttcgggaacacgctgacgtgctatctcaaagccacggccgccatcagggcggcggggctgagagactgcactatgttggtttgcggtgatgacttagtcgtcatcgctgagagcgacggcgtagaggaggacaaccgagccctccgagccttcacggaggctatgacgagatactcggctcccccaggtgacgccccgcagccagcatatgacctggaactaataacatcatgttcatccaacgtctcagtcgcgcacgacgcgacgggtaaaagggtatattacctaacccgagaccctacaactcccttggcgcgagccgcatgggagacagtccgacacactccagtcaattcctggttgggaaacatcatagtctacgctcccacaatatgggtgcgcatgatattgatgacccactttttctcaatactccagagccaggaagcccttgagaaagcactcgacttcgatatgtacggagtcacctactctatcactccgctggatttaccggcaatcattcaaagactccatggcttaagcgcgttcacgctgcacggatactctccacacgaactcaaccgggtggccggagccctcagaaaacttggggtacccccgctgagagtgtggagacatcgggcccgagcagtccgcgctaagcttatcgcccagggaggtagagccaaaatatgtggcatatacctctttaactgggcggtaaaaaccaaactcaaactcactccattgcctgccgctgccaaactcgatttatcgggttggtttacggtaggcgccggcgggggaggcatttttcacagcgtgcctcatgcccgaccccgctatttactccggtacctactcctacttacagtaggggtaggcatcttcctgctgcctgctcggtaggcagcttaacactccgaccttagggtccccttgttttttttttttttttttttttttttttttttttttttttttttcctttccttctttcctttcctaatctttctttcttggtggctccatcttagccctagtcacggctagctgtgaaaggtccgtgagccgcatgactgcagagagtgctgatactggcctctctgcagatcatgt

SEQ ID NO: 8 - DNA, 9578 bp, Hepatitis C virus
acctgctctctatgagagcaacactccaccatgaaccgctcccctgtgaggaactactgtcttcacgcagaaagcgtctagccatggcgttagtatgagtgttgtacagcctccaggaccccccctcccgggagagccatagtggtctgcggaaccggtgagtacaccggaatcgccgggatgaccgggtcctttcttggattaacccgctcaatgcccggaaatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgccgcccaatggacgttaagttcccgggtggtggccagatcgttggcggagtttacttgttgccgcgcaggggccccagattgggtgtgcgcgcgactcggaagacttcggagcggtcgcaacctcgtggaagacgccaacctatccccaaggcgcgtcgacccgagggaaggtcctgggcacaaccaggatatccatggcctctttacggtaatgagggttgtgggtgggcaggatggctcttgtccccccgtggctctcgaccgtcttggggcccaaatgatccccggcggaggtcccgcaatttgggtaaggtcatcgataccctaacctgcggcttcgccgacctcatgggatacatcccgctcgtaggcgcccccgtgggtggcgtcgccagggccctggcacatggtgtcagggctttggaggacgggatcaattatgcaacagggaatctccccggttgctccttttctatcttcctcttggcacttctttcgtgcctgactgtccccgcttcggccgttaactatcgcaatgtctcgggcatctaccatgtcaccaatgactgcccgaattcaagcatagtgtatgaggccgaccatcacatcttgcaccttccaggttgcgtgccctgcgtgagagaggggaatcagtcacgctgctgggtggcccttactcctaccgtcgcagcgccatacatcggcgcaccgcttgagtccttacggagtcatgtggatttgatggtgggggccgccactggttgctcgggtctttacatcggggacctgtgtggcggcttgttcctagttggccagatgttttcattccgaccacggcgccactggaccacccaggattgcaattgttccatctacacagggcacattacaggccacagaatggcctgggacatgatgatgaactggagtccaacaaccaccttagttctcgcccaggtcatgaggatcccaaccactctggtagacttactctctggaggccactggggtgtcctcgtgggagtggcctatttcagcatgcaggccaattgggccaaagtcatcttggtcctattcctctttgcaggggttgatgccgagactcacgtgtctggggcagcagtcggccggagtaccgccggcttggctaacctcttttcttctgggtctaagcagaatttacagctcatcaacagcaatgggagctggcatataaataggactgcccttaactgcaatgacagcttaaacactgggttcttggctagcttgttctacacccacaagtttaacagctcagggtgttccgaacggctcgcgcgctgcaagagccttgacagctacggccaaggctggggcccactcggggtcgctaacatcagcggctcgtctgatgacaggccttattgctggcactacgcgcctcggccgtgcgggattgtgccagcatccagtgtgtgtggccccgtgtattgtttcactcccagccctgtcgtggtcggtactactgatcacgtcggggtccctacttacacctggggggagaatgagactgatgtcttccttttgaactcgaccagaccgccgcatggtgcgtggtttggatgcgtgtggatgaacagtaccgggttcaccaaaacctgtggcgcccctccatgcgaggttaacaccaataatgggacctggcactgccccaccgattgcttcaggaagcatccggagactacctacgccaagtgcggatcagggccttggatcacaccgcgctgcctgattgattacccgtaccggctgtggcatttcccgtgcaccgccaacttctccgtctttaacatcaggacatttgtcggcggtatagagcatcggatgcaagcggcatgcaactggaccaggggggaagtctgtggcttggagcacagggatcgcgtagagctatcacccctgctccttaccactacagcgtggcagatcctcccctgctctttcaccactttacctgccctctccaccggcttgatccacctccaccaaaatatcgtggacgtccagtacctctatggtgttgggtctgcagtggtatcttgggcccttaagtgggaatatgtggtgctcgcgttcctgcttctcgcggacgcgagagtctctgcctgcctatggatgatgtttatggtaagtcaagttgaggcggctctgtccaacctgattaacatcaatgctgcttcagccgctggtgcccaaggcttctggtacgccatcctcttcatctgcattgtctggcatgtcaagggccggttcccagctgctgctgcctacgcagcctgcgggctgtggcccctgtttctcctgcttctgatgctgcctgagagggcttatgcatacgaccaggaagtggcagggtcccttggcggcgccatcgttgtcatgctggccattctgacactgtctccgcactacaagttatggctggctaggggattgtggtggatccaatgttttatagctaggaccgaggctgtgctgcatgtctatattccatccttcaacgtgcgcgggcctcgcgactcagtgattgttcttgcagtcctggtctgtccacacctagtatttgacatcacaaaatatcttctggccatcttagggcccctccacatactccaggcctcgctcctacgcatcccttactttgtgagggcacaagcgctggttaagatctgcagcttgttgcgtggggtagtttatggcaagtacttccaaatggtcgtgcttaaagcaggggccctgactggtacttacatctatgaccaccttactcccatgtcagattgggccgctacgggcctccgcgatttggcggtggccctagagccagttgtgttcacgcccatggagaagaaagtcatcgtctggggcgctgacaccgctgcgtgcggagacatcataaggggattacctgtttcggccaggttgggcaatgaaatcttgctcggaccagccgatacagaaacatcaaaggggtggagactccttgcccccatcacagcatacgcgcagcagacccgcggcttgttcagcaccatcgtaacgagcctcactggcagggacaccaatgagaattgtggcgaagtgcaggtcttatccaccgctacgcagtccttcctgggtactgcggttaacggcgtgatgtggaccgtctaccacggggcgggtgccaagaccatcagcggcccgaagggacctgtcaatcaaatgtacactaatgttgaccaagacttggtggggtggccagcaccccccggagtcagatctcttgctccgtgcacctgcggctcggcagacttgtatctagtcaccaggcacgcagatgtaatacccgtgcgcaggagaggagacaccagaggagctctcttgagccctagaccaatatccactcttaagggatcttccggaggtccgctgctgtgccccatgggacacgccgccggcatattccgtgcggcggtgtgtactcgaggggtagccaaggcggtagacttcgtcccggttgaatctcttgagactaccatgagatcaccagtgttcactgacaactcaacacctccagcagtgccccagacctaccaggtcgcgcacctacacgcaccaacaggaagtggcaagagcaccaaagtcccggcggcgtatgctgcccaaggctataaagtgctagtgctcaatccttcggttgcggccacactgggttttggggtatacatgtccaaggcatatggcatcgacccgaacatccggtcgggagtcaggaccatcaccacgggtgcgccagtcacgtactcaacgtatggtaagttcctggctgatggaggttgcagcggaggggcatacgacataatcatctgtgacgagtgccattccactgactccacaacgatccttggcataggcacagtcctggaccaagcggagaccgctggagtgcgcctcaccgtgctcgcgactgctactccgccagggtcagtgactacacctcattccaacatagaggaggtcgccctgccaacaacgggggaaatccccttttacggcaaggcgatccctctggagctgattaaggggggcagacatctcatcttctgccactcaaagaaaaagtgtgatgaactggccagacaactgacatctcttggtctgaatgccgtagcctactacagaggcttagacgtttcggtgattcccacgtctggggacgtcgtggtatgcgccacggacgccctcatgacgggttttaccggcgactttgactcagtgatagactgcaatacatctgtgatacagactgttgacttcggcatggaccccaccttctccatagagactacaaccgttccccaggacgcggtatcccgcagccagcggagaggccgcactggtagggggaggttgggcacataccggtatgtcaccccgggagagagaccatcaggcatgtttgacactgcagtgctttgcgagtgctacgatgccgggtgtgcctggtacgagctgacacctgctgaaaccacaacaaggctgaaagcttacttcgacacaccaggccttcctgtgtgccaagaccatctggagttctgggagagcgtctttacagggttaacccacatagacggtcatctcctatcccagaccaagcaatcgggtgagaatttcccgtatcttgttgcttaccaagccacggtgtgcgccaaggctctggcgcctccaccaagctgggacaccatgtggaagtgcctaattcgccttaagcccaccctgcacgggcccacacccctcctctacagactggggtctgtgcagaatgaagtggtgctcacccatcccatcaccaaatacatcatggcttgcatgtcagctgatctcgaggtagtgacaagtacgtgggtcttggtgggcggcgtcctggcagctctggcttcttactgtctttcagtgggcagcgtagtgattgttgggagagtcgtcctgtcgggccaacctgctgtcattcccgatcgcgaagtgctctaccaacagttcgacgaaatggaggagtgttccaaacacctcccactagtcgagcacgggttacaactggctgagcagttcaagcagaaggccttaggtctcctaaatttcgctggcaagcaagcccaagaggcaacaccagtgatccagtctaacttcgctaaacttgagcagttttgggcgaagcacatgtggaatttcatcagcggcattcaatatctcgctggactgtctaccttgccaggcaatcctgtcattgcttccctcatgtcctttactgctgctgttacaagccctctgaccacccaacaaaccctcctttttaacatcttggggggatgggtggcctcgcagattgcggctccgacggcttctaccgcattcgtcgtgagcggcttggcgggggcggcagttggcagtgtgggccttggcaaaattttggtggacattctcgccggttacggcgccggcgtagctggcgctgtggttgccttcaagatcatgaacggcgagatgccttccacagaggacttggtaaatttgctcccggccattctatcgcccggagcattggtagtgggggtggtatgcgcggcgattttgcgccgccacgtgggcccggccgaaggggctgtgcagtggatgaaccgtctaattgcgttcgcatcgcgaggcaatcacgtgtctcccacgcattacgtccctgagtccgacgcggcagcccgcgtgaccaccatactatcatccctcactgtgacatcccttctcagacgcctccacaagtggatcaatgaagattgctccaccccatgtaccgaatcttggctatgggaggtatgggattgggtctgcaccgtgctgagtgacttcaagacgtggctaaaagccaagttgctgcccctcatgccaggcatccccttcctctcatgccagaggggctataagggagagtggcgcggagatggcgtgatgcataccacatgcccctgcggagcagatctggcaggtcacatcaagaacggctcgatgagaatcaccgggccgaaaacctgcagcaacacatggcatggtaccttccccatcaatgcttacaccacaggccctggtgtacccatcccggcgccgaactacaagttcgcgctttggagggtgtccgccgaggactacgtggaggttcgcagagtgggtgatttccattatgtcaccggggtaacacaagacaacatcaagtgcccctgccaagttccggccccagagttcttcacggaagtggacggcatcaggctacaccgccacgccccgaagtgcaaacccttgctgcgggacgaagtgtcgttctcagtaggactcaattcgttcgtagtgggatcacaactcccatgcgagccagagccggacgtggcagtgctaacatccatgctgacagacccatcacacataacggcggaatcggcgcgtcggagattggctcgagggtcacgaccctcgctagctagttcctcggcgagtcagctttccgccccgtctctcaaggccacgtgtaccgctccccatgactcccctggtactgatctcctcgaggctaacctcttgtgggggtctaccgctaccagggttgagacggacgagaaggtaataatactagactcttttgagtcatgtgtgactgagccaaatgatgacagggaagtctcggttgccgcggaaatcctgcgtccgaccaagaagttccctccagcactaccgatctgggcccggccggattacaatccacctcttaccgagacgtggaagcagcaggactacaagcctccgaccgtccacgggtgcgctctgcctcccggcaagcagccccccgttcctcctcccaggaggaaacgggcggtacagctcactgagtccgttgtttctaccgctttggcagagctggccgcaaagacctttggccagtcagagccgagctcagaccgtgatacagaccttaccaccccaactgagaccacagactcgggccccatcgtcgtggatgatgcatccgatgacggatcttattcgtcaatgcctccactagagggggagcccggtgacccggacttgacatcagactcttggtccactgttagcggatcggagggcgtcgtgtgctgctcaatgtcatattcatggactggggcgcttgtaacaccttgcgcggctgaagaatcaaagctgccaattagccccctgagcaattcacttttgcgccatcacaatatggtgtatgccacgaccacccgttctgctgtgacacggcagaagaaggtgaccttcgaccgcctgcaggtggtggacagtcactacaatgaagtgcttaaggagataaaggcacgagcatccagagtgaaggcacgcttgcttaccacagaggaagcttgcgacctgacgcccccccactcagccagatcaaagttcggctacggggcgaaggatgttcggagccattcccgcaaggccattaaccacatcagctccgtgtggaaggacttgctggaagacaacaataccccaataccaacaacaatcatggccaaaaatgaggtcttcgctgtgaacccagcgaagggaggtcggaagcctgctcgcctgatcgtgtatccggatctcggggtccgggtttgcgagaagagagcgcttcacgacgtcatcaataaactgcctgaggccgtgatgggagccgcttatggcttccaatactccccagcgcagcgggtggaatttcttctgactgcttggaagtcgaagaagaccccaatggggttctcttatgatacccgctgctttgactccactgtaaccgaaaaggacatcagggtcgaggaagaggtctatcagtgttgtgacctggagcccgaagcccgcaaagtcatcaccgccctcacagagagactctatgtgggcggccctatgcacaacagcaagggagacctttgtgggtatcggagatgtcgcgcaagcggcgtctacaccaccagcttcgggaacacgctgacgtgctatctcaaagccacggccgccatcagggcggcggggctgagagactgcactatgttggtttgcggtgatgacttagtcgtcatcgctgagagcgacggcgtagaggaggacaaccgagccctccgagccttcacggaggctatgacgagatactcggctcccccaggtgacgccccgcagccagcatatgacctggaactaataacatcatgttcatccaacgtctcagtcgcgcacgacgcgacgggtaaaagggtatattacctaacccgagaccctacaactcccttggcgcgagccgcatgggagacagtccgacacactccagtcaattcctggttgggaaacatcatagtctacgctcccacaatatgggtgcgcatgatattgatgacccactttttctcaatactccagagccaggaagcccttgagaaagcactcgacttcgatatgtacggagtcacctactctatcactccgctggatttaccggcaatcattcaaagactccatggcttaagcgcgttcacgctgcacggatactctccacacgaactcaaccgggtggccggagccctcagaaaacttggggtacccccgctgagagtgtggagacatcgggcccgagcagtccgcgctaagcttatcgcccagggaggtagagccaaaatatgtggcatatacctctttaactgggcggtaaaaaccaaactcaaactcactccattgcctgccgctgccaaactcgatttatcgggttggtttacggtaggcgccggcgggggaggcatttttcacagcgtgcctcatgcccgaccccgctatttactccggtacctactcctacttacagtaggggtaggcatcttcctgctgcctgctcggtaggcagcttaacactccgaccttagggtccccttgtttttttttttttttttttttttttttttttttttttttttttttcctttccttctttcctttcctaatctttctttcttggtggctccatcttagccctagtcacggctagctgtgaaaggtccgtgagccgcatgactgcagagagtgctgatactggcctctctgcagatcatgt

SEQ ID NO: 9 - DNA, 9577 bp, Hepatitis C virus
acctgctctctatgagagcaacactccaccatgaaccgctcccctgtgaggaactactgtcttcacgcagaaagcgtctagccatggcgttagtatgagtgttgtacagcctccaggaccccccctcccgggagagccatagtggtctgcggaaccggtgagtacaccggaatcgccgggatgaccgggtcctttcttggattaacccgctcaatgcccggaaatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgccgcccaatggacgttaagttcccgggtggtggccagatcgttggcggagtttacttgttgccgcgcaggggccccagattgggtgtgcgcgcgactcggaagacttcggagcggtcgcaacctcgtggaagacgccaacctatccccaaggcgcgtcgacccgagggaaggtcctgggcacaaccaggatatccatggcctctttacggtaatgagggttgtgggtgggcaggatggctcttgtccccccgtggctctcgaccgtcttggggcccaaatgatccccggcggaggtcccgcaatttgggtaaggtcatcgataccctaacctgcggcttcgccgacctcatgggatacatcccgctcgtaggcgcccccgtgggtggcgtcgccagggccctggcacatggtgtcagggctttggaggacgggatcaattatgcaacagggaatctccccggttgctccttttctatcttcctcttggcacttctttcgtgcctgactgtccccgcttcggccgttaactatcgcaatgtctcgggcatctaccatgtcaccaatgactgcccgaattcaagcatagtgtatgaggccgaccatcacatcttgcaccttccaggttgcgtgccctgcgtgagagaggggaatcagtcacgctgctgggtggcccttactcctaccgtcgcagcgccatacatcggcgcaccgcttgagtccttacggagtcatgtggatttgatggtgggggccgccactggttgctcgggtctttacatcggggacctgtgtggcggcttgttcctagttggccagatgttttcattccgaccacggcgccactggaccacccaggattgcaattgttccatctacacagggcacattacaggccacagaatggcctgggacatgatgatgaactggagtccaacaaccaccttagttctcgcccaggtcatgaggatcccaaccactctggtagacttactctctggaggccactggggtgtcctcgtgggagtggcctatttcagcatgcaggccaattgggccaaagtcatcttggtcctattcctctttgcaggggttgatgccgagactcacgtgtctggggcagcagtcggccggagtaccgccggcttggctaacctcttttcttctgggtctaagcagaatttacagctcatcaacagcaatgggagctggcatataaataggactgcccttaactgcaatgacagcttaaacactgggttcttggctagcttgttctacacccacaagtttaacagctcagggtgttccgaacggctcgcgcgctgcaagagccttgacagctacggccaaggctggggcccactcggggtcgctaacatcagcggctcgtctgatgacaggccttattgctggcactacgcgcctcggccgtgcgggattgtgccagcatccagtgtgtgtggccccgtgtattgtttcactcccagccctgtcgtggtcggtactactgatcacgtcggggtccctacttacacctggggggagaatgagactgatgtcttccttttgaactcgaccagaccgccgcatggtgcgtggtttggatgcgtgtggatgaacagtaccgggttcaccaaaacctgtggcgcccctccatgcgaggttaacaccaataatgggacctggcactgccccaccgattgcttcaggaagcatccggagactacctacgccaagtgcggatcagggccttggatcacaccgcgctgcctgattgattacccgtaccggctgtggcatttcccgtgcaccgccaacttctccgtctttaacatcaggacatttgtcggcggtatagagcatcggatgcaagcggcatgcaactggaccaggggggaagtctgtggcttggagcacagggatcgcgtagagctatcacccctgctccttaccactacagcgtggcagatcctcccctgctctttcaccactttacctgccctctccaccggcttgatccacctccaccaaaatatcgtggacgtccagtacctctatggtgttgggtctgcagtggtatcttgggcccttaagtgggaatatgtggtgctcgcgttcctgcttctcgcggacgcgagagtctctgcctgcctatggatgatgtttatggtaagtcaagttgaggcggctctgtccaacctgattaacatcaatgctgcttcagccgctggtgcccaaggcttctggtacgccatcctcttcatctgcattgtctggcatgtcaagggccggttcccagctgctgctgcctacgcagcctgcgggctgtggcccctgtttctcctgcttctgatgctgcctgagagggcttatgcatacgaccaggaagtggcagggtcccttggcggcgccatcgttgtcatgctggccattctgacactgtctccgcactacaagttatggctggctaggggattgtggtggatccaatgttttatagctaggaccgaggctgtgctgcatgtctatattccatccttcaacgtgcgcgggcctcgcgactcagtgattgttcttgcagtcctggtctgtccacacctagtatttgacatcacaaaatatcttctggccatcttagggcccctccacatactccaggcctcgctcctacgcatcccttactttgtgagggcacaagcgctggttaagatctgcagcttgttgcgtggggtagtttatggcaagtacttccaaatggtcgtgcttaaagcaggggccctgactggtacttacatctatgaccaccttactcccatgtcagattgggccgctacgggcctccgcgatttggcggtggccctagagccagttgtgttcacgcccatggagaagaaagtcatcgtctggggcgctgacaccgctgcgtgcggagacatcataaggggattacctgtttcggccaggttgggcaatgaaatcttgctcggaccagccgatacagaaacatcaaaggggtggagactccttgcccccatcacagcatacgcgcagcagacccgcggcttgttcagcaccatcgtaacgagcctcactggcagggacaccaatgagaattgtggcgaagtgcaggtcttatccaccgctacgcagtccttcctgggtactgcggttaacggcgtgatgtggaccgtctaccacggggcgggtgccaagaccatcagcggcccgaagggacctgtcaatcaaatgtacactaatgttgaccaagacttggtggggtggccagcaccccccggagtcagatctcttgctccgtgcacctgcggctcggcagacttgtatctagtcaccaggcacgcagatgtaatacccgtgcgcaggagaggagacaccagaggagctctcttgagccctagaccaatatccactcttaagggatcttccggaggtccgctgctgtgccccatgggacacgccgccggcatattccgtgcggcggtgtgtactcgaggggtagccaaggcggtagacttcgtcccggttgaatctcttgagactaccatgagatcaccagtgttcactgacaactcaacacctccagcagtgccccagacctaccaggtcgcgcacctacacgcaccaacaggaagtggcaagagcaccaaagtcccggcggcgtatgctgcccaaggctataaagtgctagtgctcaatccttcggttgcggccacactgggttttggggtatacatgtccaaggcatatggcatcgacccgaacatccggtcgggagtcaggaccatcaccacgggtgcgccagtcacgtactcaacgtatggtaagttcctggctgatggaggttgcagcggaggggcatacgacataatcatctgtgacgagtgccattccactgactccacaacgatccttggcataggcacagtcctggaccaagcggagaccgctggagtgcgcctcaccgtgctcgcgactgctactccgccagggtcagtgactacacctcattccaacatagaggaggtcgccctgccaacaacgggggaaatccccttttacggcaaggcgatccctctggagctgattaaggggggcagacatctcatcttctgccactcaaagaaaaagtgtgatgaactggccagacaactgacatctcttggtctgaatgccgtagcctactacagaggcttagacgtttcggtgattcccacgtctggggacgtcgtggtatgcgccacggacgccctcatgacgggttttaccggcgactttgactcagtgatagactgcaatacatctgtgatacagactgttgacttcggcatggaccccaccttctccatagagactacaaccgttccccaggacgcggtatcccgcagccagcggagaggccgcactggtagggggaggttgggcacataccggtatgtcaccccgggagagagaccatcaggcatgtttgacactgcagtgctttgcgagtgctacgatgccgggtgtgcctggtacgagctgacacctgctgaaaccacaacaaggctgaaagcttacttcgacacaccaggccttcctgtgtgccaagaccatctggagttctgggagagcgtctttacagggttaacccacatagacggtcatctcctatcccagaccaagcaatcgggtgagaatttcccgtatcttgttgcttaccaagccacggtgtgcgccaaggctctggcgcctccaccaagctgggacaccatgtggaagtgcctaattcgccttaagcccaccctgcacgggcccacacccctcctctacagactggggtctgtgcagaatgaagtggtgctcacccatcccatcaccaaatacatcatggcttgcatgtcagctgatctcgaggtagtgacaagtacgtgggtcttggtgggcggcgtcctggcagctctggcttcttactgtctttcagtgggcagcgtagtgattgttgggagagtcgtcctgtcgggccaacctgctgtcattcccgatcgcgaagtgctctaccaacagttcgacgaaatggaggagtgttccaaacacctcccactagtcgagcacgggttacaactggctgagcagttcaagcagaaggccttaggtctcctaaatttcgctggcaagcaagcccaagaggcaacaccagtgatccagtctaacttcgctaaacttgagcagttttgggcgaagcacatgtggaatttcatcagcggcattcaatatctcgctggactgtctaccttgccaggcaatcctgtcattgcttccctcatgtcctttactgctgctgttacaagccctctgaccacccaacaaaccctcctttttaacatcttggggggatgggtggcctcgcagattgcggctccgacggcttctaccgcattcgtcgtgagcggcttggcgggggcggcagttggcagtgtgggccttggcaaaattttggtggacattctcgccggttacggcgccggcgtagctggcgctgtggttgccttcaagatcatgaacggcgagatgccttccacagaggacttggtaaatttgctcccggccattctatcgcccggagcattggtagtgggggtggtatgcgcggcgattttgcgccgccacgtgggcccggccgaaggggctgtgcagtggatgaaccgtctaattgcgttcgcatcgcgaggcaatcacgtgtctcccacgcattacgtccctgagtccgacgcggcagcccgcgtgaccaccatactatcatccctcactgtgacatcccttctcagacgcctccacaagtggatcaatgaagattgctccaccccatgtgccgaatcttggctatgggaggtatgggattgggtctgcaccgtgctgagtgacttcaagacgtggctaaaagccaagttgctgcccctcatgccaggcatccccttcctctcatgccagaggggctataagggagagtggcgcggagatggcgtgatgcataccacatgcccctgcggagcagatctggcaggtcacatcaagaacggctcgatgagaatcaccgggccgaaaacctgcagcaacacatggcatggtaccttccccatcaatgcttacaccacaggccctggtgtacccatcccggcgccgaactacaagttcgcgctttggagggtgtccgccgaggactacgtggaggttcgcagagtgggtgatttccattatgtcaccggggtaacacaagacaacatcaagtgcccctgccaagttccggccccagagttcttcacggaagtggacggcatcaggctacaccgccacgccccgaagtgcaaacccttgctgcgggacgaagtgtcgttctcagtaggactcaattcgttcgtagtgggatcacaactcccatgcgagccagagccggacgtggcagtgctaacatccatgctgacagacccatcacacataacggcggaatcggcgcgtcggagattggctcgagggtcacgaccctcgctagctagttcctcggcgagtcagctttccgccccgtctctcaaggccacgtgtaccgctccccatgactcccctggtactgatctcctcgaggctaacctcttgtgggggtctaccgctaccagggttgagacggacgagaaggtaataatactagactcttttgagtcatgtgtgactgagccaaatgatgacagggaagtctcggttgccgcggaaatcctgcgtccgaccaagaagttccctccagcactaccgatctgggcccggccggattacaatccacctcttaccgagacgtggaagcagcaggactacaagcctccgaccgtccacgggtgcgctctgcctcccggcaagcagccccccgttcctcctcccaggaggaaacgggcggtacagctcactgagtccgttgtttctaccgctttggcagagctggccgcaaagacctttggccagtcagagccgagctcagaccgtgatacagaccttaccaccccaactgagaccacagactcgggccccatcgtcgtggatgatgcatccgatgacggatcttattcgtcaatgcctccactagagggggagcccggtgacccggacttgacatcagactcttggtccactgttagcggatcggagggcgtcgtgtgctgctcaatgtcatattcatggactggggcgcttgtaacaccttgcgcggctgaagaatcaaagctgccaattagccccctgagcaattcacttttgcgccatcacaatatggtgtatgccacgaccacccgttctgctgtgacacggcagaagaaggtgaccttcgaccgcctgcaggtggtggacagtcactacaatgaagtgcttaaggagataaaggcacgagcatccagagtgaaggcacgcttgcttaccacagaggaagcttgcgacctgacgcccccccactcagccagatcaaagttcggctacggggcgaaggatgttcggagccattcccgcaaggccattaaccacatcagctccgtgtggaaggacttgctggaagacaacaataccccaataccaacaacaatcatggccaaaaatgaggtcttcgctgtgaacccagcgaagggaggtcggaagcctgctcgcctgatcgtgtatccggatctcggggtccgggtttgcgagaagagagcgcttcacgacgtcatcaataaactgcctgaggccgtgatgggagccgcttatggcttccaatactccccagcgcagcgggtggaatttcttctgactgcttggaagtcgaagaagaccccaatggggttctcttatgatacccgctgctttgactccactgtaaccgaaaaggacatcagggtcgaggaagaggtctatcagtgttgtgacctggagcccgaagcccgcaaagtcatcaccgccctcacagagagactctatgtgggcggccctatgcacaacagcaagggagacctttgtgggtatcggagatgtcgcgcaagcggcgtctacaccaccagcttcgggaacacgctgacgtgctatctcaaagccacggccgccatcagggcggcggggctgagagactgcactatgttggtttgcggtgatgacttagtcgtcatcgctgagagcgacggcgtagaggaggacaaccgagccctccgagccttcacggaggctatgacgagatactcggctcccccaggtgacgccccgcagccagcatatgacctggaactaataacatcatgttcatccaacgtctcagtcgcgcacgacgcgacgggtaaaagggtatattacctaacccgagaccctacaactcccttggcgcgagccgcatgggagacagtccgacacactccagtcaattcctggttgggaaacatcatagtctacgctcccacaatatgggtgcgcatgatattgatgacccactttttctcaatactccagagccaggaagcccttgagaaagcactcgacttcgatatgtacggagtcacctactctatcactccgctggatttaccggcaatcattcaaagactccatggcttaagcgcgttcacgctgcacggatactctccacacgaactcaaccgggtggccggagccctcagaaaacttggggtacccccgctgagagtgtggagacatcgggcccgagcagtccgcgctaagcttatcgcccggggaggtagagccaaaatatgtggcatatacctctttaactgggcggtaaaaaccaaactcaaactcactccattgcctgccgctgccaaactcgatttatcgggttggtttacggtaggcgccggcgggggaggcatttttcacagcgtgcctcatgcccgaccccgctatttactccggtacctactcctacttacagtaggggtaggcatcttcctgctgcctgctcggtaggcagcttaacactccgaccttagggtccccttgttttttttttttttttttttttttttttttttttttttttttttcctttccttctttcctttcctaatctttctttcttggtggctccatcttagccctagtcacggctagctgtgaaaggtccgtgagccgcatgactgcagagagtgctgatactggcctctctgcagatcatgt

SEQ ID NO: 10 - DNA, 9578 bp, Hepatitis C virus
acctgctctctatgagagcaacactccaccatgaaccgctcccctgtgaggaactactgtcttcacgcagaaagcgtctagccatggcgttagtatgagtgttgtacagcctccaggaccccccctcccgggagagccatagtggtctgcggaaccggtgagtacaccggaatcgccgggatgaccgggtcctttcttggattaacccgctcaatgcccggaaatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgccgcccaatggacgttaagttcccgggtggtggccagatcgttggcggagtttacttgttgccgcgcaggggccccagattgggtgtgcgcgcgactcggaagacttcggagcggtcgcaacctcgtggaagacgccaacctatccccaaggcgcgtcgacccgagggaaggtcctgggcacaaccaggatatccatggcctctttacggtaatgagggttgtgggtgggcaggatggctcttgtccccccgtggctctcgaccgtcttggggcccaaatgatccccggcggaggtcccgcaatttgggtaaggtcatcgataccctaacctgcggcttcgccgacctcatgggatacatcccgctcgtaggcgcccccgtgggtggcgtcgccagggccctggcacatggtgtcagggctttggaggacgggatcaattatgcaacagggaatctccccggttgctccttttctatcttcctcttggcacttctttcgtgcctgactgtccccgcttcggccgttaactatcgcaatgtctcgggcatctaccatgtcaccaatgactgcccgaattcaagcatagtgtatgaggccgaccatcacatcttgcaccttccaggttgcgtgccctgcgtgagagaggggaatcagtcacgctgctgggtggcccttactcctaccgtcgcagcgccatacatcggcgcaccgcttgagtccttacggagtcatgtggatttgatggtgggggccgccactgtttgctcgggtctttacatcggggacctgtgtggcggcttgttcctagttggccagatgttttcattccgaccacggcgccactggaccacccaggattgcaattgttccatctacacagggcacattacaggccacagaatggcctgggacatgatgatgaactggagtccaacaaccaccttagttctcgcccaggtcatgaggatcccaaccactctggtagacttactctctggaggccactggggtgtcctcgtgggagtggcctatttcagcatgcaggccaattgggccaaagtcatcttggtcctattcctctttgcaggggttgatgccgagactcacgtgtctggggcagcagtcggccggagtaccgccggcttggctaacctcttttcttctgggtctaagcagaatttacagctcatcaacagcaatgggagctggcatataaataggactgcccttaactgcaatgacagcttaaacactgggttcttggctagcttgttctacacccacaagtttaacagctcagggtgttccgaacggctcgcgtgctgcaagagccttgacagctacggccaaggctggggcccactcggggtcgctaacatcagcggctcgtctgatgacaggccttattgctggcactacgcgcctcggccgtgcgggattgtgccagcatccagtgtgtgtggccccgtgtattgtttcactcccagccctgtcgtggtcggtactactgatcacgtcggggtccctacttacacctggggggagaatgagactgatgtcttccttttgaactcgaccagaccgccgcatggtgcgtggtttggatgcgtgtggatgaacagtaccgggttcaccaaaacctgtggcgcccctccatgcgaggttaacaccaataatgggacctggcactgccccaccgattgcttcaggaagcatccggagactacctacgccaagtgcggatcagggccttggatcacaccgcgctgcctgattgattacccgtaccggctgtggcatttcccgtgcaccgccaacttctccgtctttaacatcaggacatttgtcggcggtatagagcatcggatgcaagcggcatgcaactggaccaggggggaagtctgtggcttggagcacagggatcgcgtagagctatcacccctgctccttaccactacagcgtggcagatcctcccctgctctttcaccactttacctgccctctccaccggcttgatccacctccaccaaaatatcgtggacgtccagtacctctatggtgttgggtctgcagtggtatcttgggcccttaagtgggaatatgtggtgctcgcgttcctgcttctcgcggacgcgagagtctctgcctgcctatggatgatgtttatggtaagtcaagttgaggcggctctgtccaacctgattaacatcaatgctgcttcagccgctggtgcccaaggcttctggtacgccatcctcttcatctgcattgtctggcatgtcaagggccggttcccagctgctgctgcctacgcagcctgcgggctgtggcccctgtttctcctgcttctgatgctgcctgagagggcttatgcatacgaccaggaagtggcagggtcccttggcggcgccatcgttgtcatgctggccattctgacactgtctccgcactacaagttatggctggctaggggattgtggtggatccaatattttatagctaggaccgaggctgtgctgcatgtctatattccatccttcaacgtgcgcgggcctcgcgactcagtgattgttcttgcagtcctggtctgtccacacctagtatttgacatcacaaaatatcttctggccatcttagggcccctccacatactccaggcctcgctcctacgcatcccttactttgtgagggcacaagcgctggttaagatctgcagcttgttgcgtggggtagtttatggcaagtacttccaaatggtcgtgcttaaagcaggggccctgactggtacttacatctatgaccaccttactcccatgtcagattgggccgctacgggcctccgcgatttggcggtggccctagagccagttgtgttcacgcccatggagaagaaagtcatcgtctggggcgctgacaccgctgcgtgcggagacatcataaggggattacctgtttcggccaggttgggcaatgaaatcttgctcggaccagccgatacagaaacatcaaaggggtggagactccttgcccccatcacagcatacgcgcagcagacccgcggcttgttcagcaccatcgtaacgagcctcactggcagggacaccaatgagaattgtggcgaagtgcaggtcttatccaccgctacgcagtccttcctgggtactgcggttaacggcgtgatgtggaccgtctaccacggggcgggtgccaagaccatcagcggcccgaagggacctgtcaatcaaatgtacactaatgttgaccaagacttggtggggtggccagcaccccccggagtcagatctcttgctccgtgcacctgcggctcggcagacttgtatctagtcaccaggcacgcagatgtaatacccgtgcgcaggagaggagacaccagaggagctctcttgagccctagaccaatatccactcttaagggatcttccggaggtccgctgctgtgccccatgggacacgccgccggcatattccgtgcggcggtgtgtactcgaggggtagccaaggcggtagacttcgtcccggttgaatctcttgagactaccatgagatcaccagtgttcactgacaactcaacacctccagcagtgccccagacctaccaggtcgcgcacctacacgcaccaacaggaagtggcaagagcaccaaagtcccggcggcgtatgctgcccaaggctataaagtgctagtgctcaatccttcggttgcggccacactgggttttggggtatacatgtccaaggcatatggcatcgacccgaacatccggtcgggagtcaggaccatcaccacgggtgcgccagtcacgtactcaacgtatggtaagttcctggctgatggaggttgcagcggaggggcatacgacataatcatctgtgacgagtgccattccactgactccacaacgatccttggcataggcacagtcctggaccaagcggagaccgctggagtgcgcctcaccgtgctcgcgactgctactccgccagggtcagtgactacacctcattccaacatagaggaggtcgccctgccaacaacgggggaaatccccttttacggcaaggcgatccctctggagctgattaaggggggcagacatctcatcttctgccactcaaagaaaaagtgtgatgaactggccagacaactgacatctcttggtctgaatgccgtagcctactacagaggcttagacgtttcggtgattcccacgtctggggacgtcgtggtatgcgccacggacgccctcatgacgggttttaccggcgactttgactcagtgatagactgcaatacatctgtgatacagactgttgacttcggcttggaccccaccttctccatagagactacaaccgttccccaggacgcggtatcccgcagccagcggagaggccgcactggtagggggaggttgggcacataccggtatgtcaccccgggagagagaccatcaggcatgtttgacactgcagtgctttgcgagtgctacgatgccgggtgtgcctggtacgagctgacacctgctgaaaccacaacaaggctgaaagcttacttcgacacaccaggccttcctgtgtgccaagaccatctggagttctgggagagcgtctttacagggttaacccacatagacggtcatttcctatcccagaccaagcaatcgggtgagaatttcccgtatcttgttgcttaccaagccacggtgtgcgccaaggctctggcgcctccaccaagctgggacaccatgtggaagtgcctaattcgccttaagcccaccctgcacgggcccacacccctcctctacagactggggtctgtgcagaatgaagtggtgctcacccatcccatcaccaaatacatcatggcttgcatgtcagctgatctcgaggtagtgacaagtacgtgggtcttggtgggcggcgtcctggcagctctggcttcttactgtctttcagtgggcagcgtagtgattgttgggagagtcgtcctgtcgggccaacctgctgtcattcccgatcgcgaagtgctctaccaacagttcgacgaaatggaggagtgttccaaacacctcccactagtcgagcacgggttacaactggctgagcagttcaagcagaaggccttaggtctcctaaatttcgctggcaagcaagcccaagaggcaacaccagtgatccagtctaacttcgctaaacttgagcagttttgggcgaagcacatgtggaatttcatcagcggcattcaatatctcgctggactgtctaccttgccaggcaatcctgtcattgcttccctcatgtcctttactgctgctgttacaagccctctgaccacccaacaaaccctcctttttaacatcttggggggatgggtggcctcgcagattgcggctccgacggcttctaccgcattcgtcgtgagcggcttggcgggggcggcagttggcagtgtgggccttggcaaaattttggtggacattctcgccggttacggcgccggcgtagctggcgctgtggttgccttcaagatcatgaacggcgagatgccttccacagaggacttggtaaatttgctcccggccattctatcgcccggagcattggtagtgggggtggtatgcgcggcgattttgcgccgccacgtgggcccgggcgaaggggctgtgcagtggatgaaccgtctaattgcgttcgcatcgcgaggcaatcacgtgtctcccacgcattacgtccctgagtccgacgcggcagcccgcgtgaccaccatactatcatccctcactgtgacatcccttctcagacgcctccacaagtggatcaatgaagattgctccaccccatgtgccgaatcttggctatgggaggtatgggattgggtctgcaccgtgctgagtgacttcaagacgtggctaaaagccaagttgctgcccctcatgccaggcatccccttcctctcatgccagaggggctataagggagagtggcgcggagatggcgtgatgcataccacatgcccctgcggagcagatctggcaggtcacatcaagaacggctcgatgagaatcaccgggccgaaaacctgcagcaacacatggcatggtaccttccccatcaatgcttacaccacaggccctggtgtacccatcccggcgccgaactacaagttcgcgctttggagggtgtccgccgaggactacgtggaggttcgcagagtgggtgatttccattatgtcaccggggtaacacaagacaacatcaagtgcccctgccaagttccggccccagagttcttcacggaagtggacggcatcaggctacaccgccacgccccgaagtgcaaacccttgctgcgggacgaagtgtcgttctcagtaggactcaattcgttcgtagtgggatcacaactcccatgcgagccagagccggacgtggcagtgctaacatccatgctgacagacccatcacacataacggcggaatcggcgcgtcggagattggctcgagggtcacgaccctcgctagctagttcctcggcgagtcagctttccgccccgtctctcaaggccacgtgtaccgctccccatgactcccctggtactgatctcctcgaggctaacctcttgtgggggtctaccgctaccagggttgagacggacgagaaggtaataatactagactcttttgagtcatgtgtggctgagccaaatgatgacagggaagtctcggttgccgcggaaatcctgcgtccgaccaagaagttccctccagcactaccgatctgggcccggccggattacaatccacctcttaccgagacgtggaagcagcaggactacaagcctccgaccgtccacgggtgcgctctgcctcccggcaagcagccccccgttcctcctcccaggaggaaacggacggtacagctcactgagtccgttgtttctaccgctttggcagagctggccgcaaagacctttggccagtcagagccgagctcagaccgtgatacagaccttaccaccccaactgagaccacagactcgggccccatcgtcgtggatgatgcatccgatgacggatcttattcgtcaatgcctccactagagggggagcccggtgacccggacttgacatcagactcttggtccactgttagcggatcggagggcgtcgtgtgctgctcaatgtcatattcatggactggggcgcttgtaacaccttgcgcggctgaagaatcaaagctgccaattagccccctgagcaattcacttttgcgccatcacaatatggtgtatgccacgaccacccgttctgctgtgacacggcagaagaaggtgaccttcgaccgcctgcaggtggtggacagtcactacaatgaagtgcttaaggagataaaggcacgagcatccagagtgaaggcacgcttgcttaccacagaggaagcttgcgacctgacgcccccccactcagccagatcaaagttcggctacggggcgaaggatgttcggagccattcccgcaaggccattaaccacatcagctccgtgtggaaggacttgctggaagacaacaataccccaataccaacaacaatcatggccaaaaatgaggtcttcgctgtgaacccagcgaagggaggtcggaagcctgctcgcctgatcgtgtatccggatctcggggtccgggtttgcgagaagagagcgcttcacgacgtcatcaaaaaactgcctgaggccgtgatgggagccgcttatggcttccaatactccccagcgcagcgggtggaatttcttctgactgcttggaagtcgaagaagaccccaatggggttctcttatgatacccgctgctttgactccactgtaaccgaaaaggacatcagggtcgaggaagaggtctatcagtgttgtgacctggagcccgaagcccgcaaagtcatcaccgccctcacagagagactctatgtgggcggccctatgcacaacagcaagggagacctttgtgggtatcggagatgtcgcgcaagcggcgtctacaccaccagcttcgggaacacgctgacgtgctatctcaaagccacggccgccatcagggcggcggggctgagagactgcactatgttggtttgcggtgatgacttagtcgtcatcgctgagagcgacggcgtagaggaggacaaccgagccctccgagccttcacggaggctatgacgagatactcggctcccccaggtgacgccccgcagccagcatatgacctggaactaataacatcatgttcatccaacgtctcagtcgcgcacgacgtgacgggtaaaagggtatattacctaacccgagaccctacaactcccttggcgcgagccgcatgggagacagtccgacacactccagtcaattcctggttgggaaacatcatagtctacgctcccacaatatgggtgcgcatgatattgatgacccactttttctcaatactccagagccaggaagcccttgagaaagcactcgacttcgatatgtacggagtcacctactctatcactccgctggatttaccggcaatcattcaaagactccatggcttaagcgcgttcacgctgcacggatactctccacacgaactcaaccgggtggccggagccctcagaaaacttggggtacccccgctgagagtgtggagacatcgggcccgagcagtccgcgctaagcttatcgcccggggaggtagagccaaaatatgtggcatatacctctttaactgggcggtaaaaaccaaactcaaactcactccattgcctgccgctgccaaactcgatttatcgggttggtttacggtaggcgccggcgggggaggcatttttcacagcgtgtctcatgcccgaccccgctatttactccggtacctactcctacttacagtaggggtaggcatcttcctgctgcctgctcggtaggcagcttaacactccgaccttagggtccccttgtttttttttttttttttttttttttttttttttttttttttttttcctttccttctttcctttcctaatctttctttcttggtggctccatcttagccctagtcacggctagctgtgaaaggtccgtgagccgcatgactgcagagagtgctgatactggcctctctgcagatcatgt

SEQ ID NO: 11 - DNA, 20 bp, Artificial Sequence
  RT-primer
ctaaggtcggagtgttaagc

SEQ ID NO: 12 - DNA, 20 bp, Artificial Sequence
  PCR-primer
tgcctgatagggtgcttgcg

SEQ ID NO: 13 - DNA, 21 bp, Artificial Sequence
  PCR-primer
aggtcggagtgttaagctgcc

(19) United States
(12) Patent Application Publication
(10) Pub. No.: US 2024/0327801 A1
(43) Pub. Date: Oct. 3, 2024

(54) EFFICIENT CELL CULTURE SYSTEM FOR HEPATITIS C VIRUS GENOTYPE 4a

(71) Applicant: Hvidovre Hospital, Hvidovre (DK)

(72) Inventors: Martin Schou Pedersen, Hvidovre (DK); Jens Bukh, Hvidovre (DK); Long Van Pham, Hvidovre (DK); Santseharay Ramirez Almeida, Hvidovre (DK); Ulrik Fahnøe, Hvidovre (DK); Carlota Fernandez-Antunez, Hvidovre (DK); Daryl Grant Humes, Hvidovre (DK); Kristian Schønning, Hvidovre (DK)

(21) Appl. No.: 18/276,852

(22) PCT Filed: Feb. 11, 2022
(86) PCT No.: PCT/DK2022/050026

§ 371 Date: Aug. 10, 2023

(30) Foreign Application Priority Data
Feb. 15, 2021 (DK) PA 2021 70066

(51) Int. Cl. C12N 7/00; A61K 39/00

(52) U.S. Cl. CPC C12N 7/00; A61K 39/00; A61K 2039/525; C12N 2770/24221; C12N 2770/24234; C12N 2770/24251

(57) ABSTRACT

The present invention relates to nucleic acid sequences that encode hepatitis C viruses (HCV) of genotype da that are useful in the fundamental research of HCV as well as in the search of antivirals and vaccines against HCV. In particular, the present invention relates to nucleic acid sequences that comprise HCV, which are capable of expressing the virus when transfected into cells and are capable of replication or infectivity in cultured cells.

### TECHNICAL FIELD OF THE INVENTION

The present invention provides infectious recombinant hepatitis C genotype 4a viruses (HCV), and vectors, cells and animals comprising the same. The present invention provides methods of producing the infectious recombinant HCV genotype 4a, and their use in identifying anti-HCV therapeutics including use in vaccines.

### BACKGROUND OF THE INVENTION

Hepatitis C virus (HCV) infection remains an important health threat, with 71 million chronically infected people, resulting in 400 thousand deaths yearly. The use of direct-acting antivirals (DAA) targeting the viral nonstructural 3 protease (NS3P), NS5A, and NS5B-polymerase increased cure rates to >90%. However, emergence of resistance-associated substitutions (RAS) compromises efficacy of DAA regimens (Pham et al, 2018; Ramirez et al, 2016). Treatment failures have been widely reported and will increase as patients are treated worldwide.

HCV shows extensive genetic diversity with eight major genotypes and >90 subtypes. Genotype 4 represents ˜8% of infections worldwide, being highly predominant in the Middle-East and North- and Central Africa. Particularly, ˜93% of >5 million HCV infections in Egypt were caused by genotype 4. This genotype is highly heterogeneous with >18 recognized subtypes. In addition, due to human migration and increasing transmission among intravenous drug users and individuals with high-risk sexual practices, the prevalence of genotype 4 is currently increasing in Europe. Subtype 4a is common, particularly in Egypt, the origin of prototype 4a strain ED43 (Gottwein et al, 2010).

Approved DAA-regimens are highly efficient against genotype 4. However, high rates of treatment failure with preexisting and emergent RASs were recently reported in subsets of genotype 4 infected patients. In Egypt alone, ˜2.4 million patients have been treated with DAAs, in particular various NS5A-inhibitors combined with the polymerase-inhibitor sofosbuvir. Although the sustained virologic response (SVR) rates were >90%, treatment failures were previously reported. Antiviral resistance will not necessarily alter the current treatment guidelines, but it could potentially affect treatment options in the future. Although DAA-resistance occurs at low prevalence, the extensive cross-resistance between the same molecular classes of drugs could limit future treatment options, especially since no additional antivirals are being developed for the treatment of HCV. Thus, generating new knowledge on viral resistance to DAAs is of great importance to prevent treatment failure in the future and to avoid the emergence and transmission of DAA-resistant viruses to highly exposed populations. This effort will require detailed understanding of the mechanisms underlying emergence of RASs.

In addition, prophylactic HCV vaccines will be essential for preventing transmission globally. Efficient infectious cell culture systems representing the major HCV genotypes can play an important role in the development and testing of vaccine candidates. Vaccine candidates based on inactivated whole-virus-particles are dependent on the efficient production of the virus in cell culture, which for HCV can only be achieved after virus adaptation, thus understanding such processes is fundamental to generate relevant candidates. In addition, evaluation of the ability of vaccines to induce broad cross-genotype neutralizing antibodies requires the establishment of culture systems representing the genetic heterogeneity of HCV, and here infectious full-length systems are most relevant since they recapitulate the entire viral life cycle (Ramirez et al. 2018; Mathiesen et al. 2015).

Efficient full-length infectious culture systems have been developed for selected strains of genotypes 1a, 2a, 2b, 3a, and 6a after complex adaptation processes (Pham et al. 2018; Ramirez et al. 2016; Li et al. 2012a; Li et al. 2015; Li et al 2012b; Ramirez et al. 2014). For genotype 4, a full-length cell culture system has recently been reported, but with limited propagation in Huh7.5 cells (Watanabe et al. 2020), thus a high titer system is required for most studies on the viral life-cycle, antivirals and vaccine development. Here, we aimed at developing a robust and efficient (high infectivity titer) full-length infectious system for HCV genotype 4a, permitting in-depth analysis of evolutionary networks underlying the emergence of DAA-resistance and assessments of the efficacy and barrier to resistance of clinically relevant DAA-regimens.

Hence, an improved robust and efficient (high infectivity titer) infectious system for HCV genotype 4a would be advantageous such as a full-length infectious system. In particular, an infectious system for HCV genotype 4a for permitting in-depth analysis of evolutionary networks underlying the emergence of DAA-resistance and assessments of the efficacy and barrier to resistance of clinically relevant DAA-regimens would be advantageous.

### SUMMARY OF THE INVENTION

Thus, an object of the present invention relates to the provision of robust and efficient infectious systems for HCV genotype 4a.

In particular, it is an object of the present invention to provide a full-length infectious system that permits in-depth analysis of the emergence of DAA-resistance.

The present invention has adapted a genotype 4a strain to efficient growth in-vitro, permitting relevant studies of viral pathogenesis, HCV inhibitor-efficacy, DAA resistance, and vaccine development.

Thus, one aspect of the invention relates to an isolated nucleic acid molecule which encodes human hepatitis C virus of genotype 4a, strain ED43, wherein said molecule encodes an amino acid sequence with a sequence identity of at least 95% to that of SEQ ID NO: 1 or a fragment hereof, and wherein the molecule
comprises the following adaptive mutations: 11291V, S1465G, A1672S, A1786V, T1822A, T1865A, S1870N, D2413G, D2545E, D2675E, K2797R, E2806T, A2916V, D2976G, Y2978F, M2981V, L2991R and C2992Y according to SEQ ID NO: 1;
optionally, further comprises the following adaptive mutation T827A according to SEQ ID NO: 1; and
one of the following groups of additional adaptive mutations:
   a) Q2931R according to SEQ ID NO: 1;
   b) V271G, C458R, Y848C, L1466M, F1572L, G1909A, A2257T, T2329A, K2597N, V2793A, Q2931R and S2982P according to SEQ ID NO: 1; or
   c) V271G, C458R, Y848C, L1466M, F1572L, G1909A, A1973T, A2257T, T2329A, K2597N, V2793A, and S2982P according to SEQ ID NO: 1.

Another aspect of the present invention relates to a composition comprising a nucleic acid molecule as described herein suspended in a suitable amount of a pharmaceutical acceptable diluent or excipient.

Yet another aspect of the present invention is to provide a cassette vector for cloning viral genomes, comprising, inserted therein, the nucleic acid molecule as described herein and having an active promoter upstream thereof.

Still another aspect of the present invention relates to a cell comprising the nucleic acid molecule as described herein, the composition as described herein or the cassette vector as described herein.

A still further aspect of the present invention relates to a method for producing a hepatitis C virus particle, comprising culturing a cell as described herein to allow the cell to produce the virus.

Yet another aspect of the present invention relates to a hepatitis C virus particle obtainable by the method as described herein.

Still another aspect of the present invention relates to a hepatitis C vaccine comprising a hepatitis C virus particle as described herein or a part thereof.

A still further aspect of the present invention relates to a method for producing a hepatitis C virus vaccine comprising using a hepatitis C virus particle obtained as described herein as an antigen.

An even further aspect relates to an antibody against the hepatitis C virus particle as described herein.

Still another aspect of the present invention relates to a method for producing a cell, which replicates human hepatitis C virus and optionally produces a virus particle comprising introducing a nucleic acid molecule as described herein into a cell.

A further aspect of the present invention relates to a cell obtainable by the method as described herein.

An even further aspect of the present invention relates to a method for producing a hepatitis C virus particle, comprising culturing a cell as described herein to allow the cell to produce the virus.

A still further aspect of the present invention relates to a method for producing a hepatitis C virus replication system, comprising culturing a cell as described herein to allow the cell to replicate the virus genome.

Yet another aspect of the present invention relates to a method for in vitro producing a hepatitis C virus-infected cell comprising culturing a cell as described herein and infecting other cells with the produced virus particle in the culture.

A still further aspect of the present invention relates to a method for screening an anti-hepatitis C virus substance, comprising
a) culturing at least one selected from the group consisting of a cell comprising the nucleic acid molecule as described herein, a cell as described herein, a hepatitis C virus particle obtainable from the method as described herein and a hepatitis C virus replication system obtainable from the method as described herein together with a hepatitis C virus permissive cell, and
b) detecting the replicating RNA or the virus particles in the resulting culture.

### BRIEF DESCRIPTION OF THE FIGURES

FIG. 1 shows a full-length HCV genotype 4a infectious cell-culture system. (A) Schematic overview of the culture adaptation process of strain ED43. Substitutions introduced into the full-length recombinant ED43-20m are shown in black. Adaptive substitutions identified during passage of the ED43-20m virus are indicated in ED43-31m, ED43-31m_opt and ED43cc. (B) HCV infectivity (bars) determined by FFU assays and shown by mean of triplicates±SEM (left y-axis; break indicates the cut-off of the assay), and emergence of substitutions for ED43-20m (lines) during adaptation following transfection (T; samples not available from days 0 to 27) and serial passages (P1, 2, 3, etc.). Only substitutions with single nucleotide polymorphism (SNP) frequencies >20% at any time-point are shown (right y-axis). For substitutions that emerged with similar patterns, means±SEM are shown. The substitutions with similar SNP frequencies are grouped. (C) Clonal ORF sequence analysis of ED43-20m virus obtained from the 6^{th}- and 10^{th} passages; phylogeny was generated as described (Jensen et al. 2019). Subsequently, ancestral reconstruction was performed based on alignment and phylogeny to predict the appearance of respective substitutions. Only substitutions indicated in (B) are shown. (D,E) Infectivity titers (y-axis) of specific ED43 recombinant viruses at indicated days (x-axis) after transfection of Huh7.5 cells. J6/JFH1 was included as a control. #The data were obtained from a separate experiment with similar J6/JFH1 titers. Statistical significance (p<0.001, two-way ANOVA) is carried out for indicated samples and shown with asterisks (***).

FIG. 2 shows substitution networks emerging in NS3P domain under protease inhibitor treatments. (A-C) ED43 full-length cultures were treated with protease inhibitors paritaprevir (A), grazoprevir (B) and glecaprevir (C) and analyzed by NGS. The percentage of HCV-antigen positive cells was determined by immunostaining (line graph, left y-axis). The distribution of haplotypes (bar graphs, right y-axis) was determined by linkage analysis and indicated by different patterns. Treatments were initiated with concentrations equivalent to 8×-EC₅₀, then concentrations were increased to 128×-EC₅₀ at day 16 (paritaprevir), 70 (grazoprevir) and 63 (glecaprevir). Only haplotypes constituting ≥2% of the viral population are shown. Supernatant samples from the last treatment time-points were used for passage. P1 and P2: the first- and second-passages. (D) Efficacy of glecaprevir against indicated ED43 full-length DAA escape viruses. Values are means of triplicates±SEM. (E) EC₅₀ values and 95% confidence intervals (CI) of indicated escape viruses were calculated from data shown in (D) and FIG. 11. Fold changes in EC₅₀ values were compared to the original ED43. (F) Fitness of ED43 recombinant virus harboring NS3P RASs. For details, see FIG. 1D,E legend. The corresponding drug targets and protein specific numbers are indicated for RASs. Other substitutions are shown with polyprotein specific numbers. #The data were obtained from a separate experiment with similar titers of the original virus.

FIG. 3 shows substitution networks emerging in NS5A domain I under treatments with NS5A inhibitors. (A-E) ED43 full-length cultures were treated with ombitasvir (A), elbasvir (B), ledipasvir (C), velpatasvir (D), and pibrentasvir (E). Panel A-C: Inhibitor concentration: 100×-EC₅₀. Panel D: Initial concentration: 10×-EC₅₀; increased to 100×-EC₅₀ at day 28. Panel E: Treatment initiated at 5×-EC₅₀, then increased to 10×- and 100×-EC₅₀ at day 33 and 40, respectively. (F) Efficacy of pibrentasvir against ED43 full-length DAA escape viruses. Values are means of triplicates±SEM. (G) EC₅₀ values and 95% CI of indicated escape viruses were calculated from data shown in (F) and FIG. 13. (H) Fitness of ED43 recombinant viruses harboring NS5A RASs. ^{#}The data were obtained from the experiment shown in FIG. 2F. For details, see FIG. 2 legend.

FIG. 4 shows substitution networks emerging in NS5B-palm domain under sofosbuvir treatment. (A) ED43 full-length cultures were treated with sofosbuvir and analyzed by NGS. Treatment was initiated with 1×-EC₅₀, then increased to 2×-EC₅₀ at day 9. (B) Efficacy of sofosbuvir against indicated ED43 full-length DAA escape viruses. Values are means of triplicates±SEM. (C) Fitness of ED43 recombinant viruses harboring RAS NS5B-S282T. For details, see FIG. 2 legend.

FIG. 5 shows efficacy of DAA combinations containing PIs and NS5A inhibitors against ED43 escape viruses. (A-C) For each DAA combination, concentrations of 5×-EC₅₀ of NS5A inhibitors were used in combination with either 2×-, 4×- or 8×-EC₅₀ of corresponding PIs. Black dashed lines indicate the time when the treatments were stopped. (D-F) NGS analysis of complete ORF sequences of viruses that escaped from DAA combinations. Only SNP of non-synonymous mutations with frequencies >20% in pre-and/or post-treatments are shown. These SNPs showed a frequency of <20% in the original virus that underwent treatments with single inhibitors (FIGS. 12 and 14). The putative RASs are shown in grey. The corresponding drug targets and protein specific numbers are indicated for RASs.

FIG. 6 shows efficacy of DAA combinations containing NS5A inhibitors and sofosbuvir against ED43 escape viruses. (A,B) For each DAA combination, concentrations of 5×-EC₅₀ of NS5A inhibitors were used in combination with either 1×- or 2×-EC₅₀ of sofosbuvir. (C,D) NGS analysis of complete ORF sequences of viruses after combination treatments. For details, see FIG. 5 legend.

FIG. 7 shows evaluation of glecaprevir/pibrentasvir as a re-treatment option against ED43 DAA escape viruses. HCV infections (A) and NGS analysis of complete ORF sequences of viruses (B) after treatments with glecaprevir/pibrentasvir. DAA escape viruses that were not eradicated by other investigated DAA combinations, were all treated with glecaprevir/pibrentasvir. Concentrations of 4×-EC₅₀ of glecaprevir in combination with 5×-EC₅₀ of pibrentasvir were used. For details, see FIG. 5 legend.

FIG. 8 shows alignment of different HCV sequences. (A) Alignment of HCV NS5B genotypes 1-4 and 6. NS5B sequences from JFH1cc (2a) (Wakita et al. 2005), J6cc (2a) (Li et al. 2012b), J8cc (2b) (Li et al. 2012b), DH8cc (2b) (Ramirez et al. 2014), TNcc (1a) (Li et al. 2012a), H77Ccc (1a) (Li et al. 2015), HCV1cc (1a) (Li et al. 2015), DBN3acc (3a) (Ramirez et al. 2016), HK2cc (6a) (Pham et al. 2018), and HK6acc (6a) (Pham et al. 2018) viruses were aligned with corresponding sequence of ED43 (4a) (Gottwein et al. 2010). Black boxes indicate residues of ED43, which are different from JFH1 and all other culture strains included from our prior studies. (B) Alignment of HCV sequences from ED43 consensus clone and ED43cc. The polyprotein sequence of ED43 consensus (Gottwein et al. 2010) was aligned with the corresponding sequence of ED43cc virus. The black boxes indicate the NS3/4A (aa 1027-1711), NS5A (aa 1973-2411), and NS5B (aa 2418-3008) sequences, respectively.

FIG. 9 shows viability of the recombinant ED43-22m following transfection of Huh7.5 cells and emergence of substitutions during virus passages. ED43-22m originated from ED43-20m with the addition of substitutions L1466M (NS3) and K2597N (NS5B), the first dominant changes emerging during passage of ED43-20m viruses (see FIG. 1B). (A) The infectivity titers were determined by FFU assays and shown by mean of triplicates±SEM (y-axis). J6/JFH1 was included as a control. Y-axis break indicates cut-off of the assay. (B) NGS analysis of recovered viruses. Only substitutions that developed in >20% of the virus population at any time points are shown. Samples were not collected during the transfection (T). Black dashed lines showed samples taken during the indicated cell-free passages of supernatant viruses (P1, 2, 3, etc.).

FIG. 10 shows evolution of substitutions and synonymous mutations in culture adaptation of ED43 full-length recombinant ED43-20m, determined by NGS. Only SNPs with frequencies of >20% at any time points are shown. For SNPs that emerged with similar patterns, means±SEM are shown instead. Black dashed lines showed samples taken during the indicated passages (P1, 2, 3, etc.). (A,B) SNP frequencies of substitutions (A) and synonymous mutations (B), determined from intracellular viral RNA. The numbers refer to amino acid positions of the ED43 polyprotein (A) or nucleotide positions of the ED43 genome (B). Samples were not collected during the transfection (T) and serial passages P2-5. (C) SNP frequency of synonymous mutations determined from extracellular viral RNAs. The numbers refer to nucleotide positions of the ED43 genome. Samples were not collected during the transfection (T) from days 0 to 27. See also FIG. 1B.

FIG. 11 shows efficacy of protease inhibitors (PIs) against HCV genotype 4a (ED43) full-length culture viruses. Huh7.5 cells were seeded on 96-well plates overnight, then infected with the indicated viruses for 24 hours. The cells were subsequently treated with specific inhibitors for an additional 48 hours before analysis as described (Pham et al. 2018; Gottwein et al. 2011). Values are means of triplicates±SEM. The original D43 virus was used in these two experiments as a control. TN (1a) virus was included for comparison. See also FIG. 2C,E.

FIG. 12 shows NGS analysis of complete ORFs of ED43 escape viruses from treatments with protease inhibitors. (A-C) The frequencies of non-synonymous mutations in ORFs of the escape viruses under treatments with paritaprevir (A), grazoprevir (B) and glecaprevir (C), were analyzed by NGS as described (Pham et al. 2018). Only SNPs forming less than 20% of the genome population at day 0 that then emerged to represent more than 20% at least one-time point during treatment are shown. The putative RASs are shown in black with protein-specific numbers (in parentheses). Dashed line indicates HCV-antigen positive cells during the treatment. Shaded backgrounds indicate 1^{st}- and 2^{nd} passages without drugs (drug-free) using the samples from the last timepoint in each treatment experiment i.e. the area between the first vertical dotted line to the second dotted line (P1) indicates 1^{st} passage while the area between the second dotted line (P1) and third dotted line (P2) indicates 2^{nd} passage. See also FIG. 2A-C.

FIG. 13 shows efficacy of NS5A inhibitors against HCV genotype 4a (ED43) viruses. Huhu7.5 cells were seeded on 96-well plates overnight, then infected with the indicated viruses for 24 hours. The cells were subsequently treated with specific inhibitors for an additional 48 hours before analysis as described (Pham et al. 2018; Gottwein et al. 2011). Values are means of triplicates±SEM. The original ED43 virus was used as a control. TN (1a) virus was included for comparison. See also FIG. 3D,E.

FIG. 14 shows NGS analysis of complete ORFs of ED43 escape viruses from treatments with NS5A inhibitors. (A-E) The frequencies of non-synonymous mutations in ORFs of the escape viruses under treatments with ombitasvir (A), elbasvir (B), ledipasvir (C), velpatasvir (D) and pibrentasvir (E), were analyzed by NGS as described (Pham et al. 2018). Only SNPs forming less than 20% of the genome population at day 0 that then emerged to represent more than 20% at least one-time point during treatment are shown. The putative RASs are shown in black with protein-specific numbers (in parentheses). Dashed line indicates HCV antigen positive cells during the treatment.

Shaded backgrounds indicate 1^{st}- and 2^{nd} passages without drugs (drug-free) using the samples from the last timepoint in each treatment experiment i.e. the area between the first vertical dotted line to the second dotted line (P1) indicates 1^{st} passage while the area between the second dotted line (P1) and third dotted line (P2) indicates 2^{nd} passage. See also FIG. 3A-E.

FIG. 15 shows NGS analysis of complete ORFs of ED43 escape viruses from treatments with sofosbuvir. The frequencies of non-synonymous mutations in ORFs of the escape viruses under treatment with sofosbuvir, were analyzed by NGS as described (Pham et al. 2018). Only SNPs forming less than 20% of the genome population at day 0 that then emerged to represent more than 20% at least one-time point during treatment are shown. The putative RASs are shown in black with protein-specific numbers (in parentheses). Dashed line indicates HCV-antigen positive cells during the treatment.

Shaded backgrounds indicate 1st- and 2^{nd} passages without drugs (drug-free) using the samples from the last timepoint in each treatment experiment i.e. the area between the first vertical dotted line to the second dotted line (P1) indicates 1^{st} passage while the area between the second dotted line (P1) and third dotted line (P2) indicates 2^{nd} passage. See also FIG. 4A.

FIG. 16 shows distributions of haplotypes in viruses after treatment with DAA combinations containing PIs and NS5A inhibitors. (A-C) Linkage analysis showed distributions of haplotypes in viruses that escaped from treatments with paritaprevir/ombitasvir (A), grazoprevir/elbasvir (B), and glecaprevir/pibrentasvir (C). For each combination, two different concentrations of PIs were used as outlined in FIG. 5. Only haplotypes accounting for ≥2% of the viral population are shown. PAR, OMB, GRA, ELB, GLE, and PIB: paritaprevir, ombitasvir, grazoprevir, elbasvir, glecaprevir, and pibrentasvir, respectively. PAResc, GRAesc, GLEesc, OMBesc, ELBesc, and PIBesc: the virus that escaped from single treatments with paritaprevir, grazoprevir, glecaprevir (as shown in FIG. 2), ombitasvir, elbasvir, and pibrentasvir (as shown in FIG. 3), respectively. See also FIG. 5.

FIG. 17 shows distributions of haplotypes in viruses after treatment with DAA combinations containing NS5A inhibitors and sofosbuvir. (A,B) Linkage analysis showed distributions of viral haplotypes after treatments with ledipasvir/sofosbuvir (A) and velpatasvir/sofosbuvir (B). For each DAA combination, the concentrations of 5×-EC₅₀ of NS5A inhibitors were used in combination with either 1×- or 2×-EC₅₀ of sofosbuvir. For details, see legend of FIG. 16. LED, VEL, and SOF: ledipasvir, velpatasvir, and sofosbuvir, respectively. LEDesc, VELesc, and SOFesc: the virus that escaped from single treatments with ledipasvir, velpatasvir (as shown in FIG. 3), and sofosbuvir (as shown in FIG. 4), respectively. See also FIG. 6.

FIG. 18 shows linkage analysis showing distributions of viral haplotypes after treatment with DAA combination glecaprevir/pibrentasvir. The concentrations of 4×-EC₅₀ of glecaprevir in combination with 5×-EC₅₀ of pibrentasvir were used for treatments. For details, see FIG. 16 legend. GRAesc, PAResc: the virus escaped from single treatments with grazoprevir, paritaprevir (as shown in FIG. 2), respectively. See also FIG. 7.

The present invention will now be described in more detail in the following.

### DETAILED DESCRIPTION OF THE INVENTION

The present invention advantageously provides hepatitis C virus (HCV) of genotype 4a nucleotide sequences capable of replication, expression of functional HCV proteins, and infection in cells for development of antiviral therapeutics, diagnostics, and vaccines.

Nucleic Acid Molecules (cDNA Clones and RNA Transcripts)

The present invention is directed towards an isolated nucleic acid molecule which encodes human hepatitis C virus of genotype 4a, strain ED43, wherein the said molecule encodes an amino acid sequence with a sequence identity of at least 95% to that of

SEQ ID NO: 1 or a fragment hereof, and wherein the molecule
comprises the following adaptive mutations: 11291V, S1465G, A1672S, A1786V, T1822A, T1865A, S1870N, D2413G, D2545E, D2675E, K2797R, E2806T, A2916V, D2976G, Y2978F, M2981V, L2991R and C2992Y according to SEQ ID NO: 1;
optionally, further comprises the following adaptive mutation T827A according to SEQ ID NO: 1; and
one of the following groups of additional adaptive mutations:
   a) Q2931R according to SEQ ID NO: 1;
   b) V271G, C458R, Y848C, L1466M, F1572L, G1909A, A2257T, T2329A, K2597N, V2793A, Q2931R and S2982P according to SEQ ID NO: 1; or
   c) V271G, C458R, Y848C, L1466M, F1572L, G1909A, A1973T, A2257T, T2329A, K2597N, V2793A, and S2982P according to SEQ ID NO: 1.

In one embodiment, the isolated nucleic acid molecule further comprises an adaptive mutation in the 5′UTR region, said adaptive mutation being G38A according to SEQ ID NO: 6. In a still further embodiment, the molecule comprises said additional adaptive mutations of group c).

In one embodiment, the isolated nucleic acid molecule which encodes human hepatitis C virus of genotype 4a, strain ED43, wherein the said molecule encodes an amino acid sequence with a sequence identity of at least 95% to that of SEQ ID NO: 1 or a fragment hereof, and wherein the molecule
comprises the following adaptive mutations: T827A, I1291V, S1465G, A1672S, A1786V, T1822A, T1865A, S1870N, D2413G, D2545E, D2675E, K2797R, E2806T, A2916V, D2976G, Y2978F, M2981V, L2991R and C2992Y according to SEQ ID NO: 1; and
one of the following groups of additional adaptive mutations:
   a) Q2931R according to SEQ ID NO: 1;
   b) V271G, C458R, Y848C, L1466M, F1572L, G1909A, A2257T, T2329A, K2597N, V2793A, Q2931R and S2982P according to SEQ ID NO: 1; or
   c) V271G, C458R, Y848C, L1466M, F1572L, G1909A, A1973T, A2257T, T2329A, K2597N, V2793A, and S2982P according to SEQ ID NO: 1.

In another embodiment, the nucleic acid molecule which encodes human hepatitis C virus of genotype 4a, strain ED43, wherein the said molecule encodes an amino acid sequence with a sequence identity of at least 95% to that of SEQ ID NO: 1 or a fragment hereof, and wherein the molecule comprises the following adaptive mutations: T827A, I1291V, S1465G, A1672S, A1786V, T1822A, T1865A, S1870N, D2413G, D2545E, D2675E, K2797R, E2806T, A2916V, Q2931R, D2976G, Y2978F, M2981V, L2991R and C2992Y according to SEQ ID NO: 1.

In a further embodiment, the nucleic acid molecule encodes human hepatitis C virus of genotype 4a, strain ED43 of SEQ ID NO: 1, wherein the molecule comprises the following adaptive mutations: T827A, I1291V, S1465G, A1672S, A1786V, T1822A, T1865A, S1870N, D2413G, D2545E, D2675E, K2797R, E2806T, A2916V, Q2931R, D2976G, Y2978F, M2981V, L2991R and C2992Y according to SEQ ID NO: 1.

In another embodiment, the nucleic acid molecule which encodes human hepatitis C virus of genotype 4a, strain ED43, wherein the said molecule encodes an amino acid sequence with a sequence identity of at least 95% to that of SEQ ID NO: 1 or a fragment hereof, and wherein the molecule comprises the following adaptive mutations: V271G, C458R, T827A, Y848C, I1291V, S1465G, L1466M, F1572L, A1672S, A1786V, T1822A, T1865A, S1870N, G1909A, A2257T, T2329A, D2413G, D2545E, K2597N, D2675E, V2793A, K2797R, E2806T, A2916V, Q2931R, D2976G, Y2978F, M2981V, S2982P, L2991R and C2992Y according to SEQ ID NO: 1.

In a further embodiment, the nucleic acid molecule, which encodes human hepatitis C virus of genotype 4a, strain ED43 of SEQ ID NO: 1, wherein the molecule comprises the following adaptive mutations: V271G, C458R, T827A, Y848C, I1291V, S1465G, L1466M, F1572L, A1672S, A1786V, T1822A, T1865A, S1870N, G1909A, A2257T, T2329A, D2413G, D2545E, K2597N, D2675E, V2793A, K2797R, E2806T, A2916V, Q2931R, D2976G, Y2978F, M2981V, S2982P, L2991R and C2992Y according to SEQ ID NO: 1.

In another embodiment, the nucleic acid molecule which encodes human hepatitis C virus of genotype 4a, strain ED43, wherein the said molecule encodes an amino acid sequence with a sequence identity of at least 95% to that of SEQ ID NO: 1 or a fragment hereof, and wherein the molecule comprises the following adaptive mutations: V271G, C458R, T827A, Y848C, I1291V, S1465G, L1466M, F1572L, A1672S, A1786V, T1822A, T1865A, S1870N, G1909A, A1973T, A2257T, T2329A, D2413G, D2545E, K2597N, D2675E, V2793A, K2797R, E2806T, A2916V, D2976G, Y2978F, M2981V, S2982P, L2991R and C2992Y according to SEQ ID NO: 1.

In a further embodiment, the nucleic acid molecule which encodes human hepatitis C virus of genotype 4a, strain ED43 of SEQ ID NO: 1, and wherein the molecule comprises the following adaptive mutations: V271G, C458R, T827A, Y848C, I1291V, S1465G, L1466M, F1572L, A1672S, A1786V, T1822A, T1865A, S1870N, G1909A, A1973T, A2257T, T2329A, D2413G, D2545E, K2597N, D2675E, V2793A, K2797R, E2806T, A2916V, D2976G, Y2978F, M2981V, S2982P, L2991R and C2992Y according to SEQ ID NO: 1.

In another embodiment, the nucleic acid molecule which encodes human hepatitis C virus of genotype 4a, strain ED43, wherein the said molecule encodes an amino acid sequence with a sequence identity of at least 95% to that of SEQ ID NO: 1 or a fragment hereof, and wherein the molecule comprises the following adaptive mutations: V271G, C458R, T827A, Y848C, I1291V, S1465G, L1466M, F1572L, A1672S, A1786V, T1822A, T1865A, S1870N, G1909A, A1973T, A2257T, T2329A, D2413G, D2545E, K2597N, D2675E, V2793A, K2797R, E2806T, A2916V, D2976G, Y2978F, M2981V, S2982P, L2991R and C2992Y according to SEQ ID NO: 1 and wherein the nucleic acid molecule further comprises an adaptive mutation in the 5′UTR region, said adaptive mutation being G38A according to SEQ ID NO: 6.

In a further embodiment, the nucleic acid molecule which encodes human hepatitis C virus of genotype 4a, strain ED43 of SEQ ID NO: 1, and wherein the molecule comprises the following adaptive mutations: V271G, C458R, T827A, Y848C, I1291V, S1465G, L1466M, F1572L, A1672S, A1786V, T1822A, T1865A, S1870N, G1909A, A1973T, A2257T, T2329A, D2413G, D2545E, K2597N, D2675E, V2793A, K2797R, E2806T, A2916V, D2976G, Y2978F, M2981V, S2982P, L2991R and C2992Y according to SEQ ID NO: 1 and wherein the nucleic acid molecule further comprises an adaptive mutation in the 5′UTR region, said adaptive mutation being G38A according to SEQ ID NO: 6.

In a further embodiment, the nucleic acid molecule as described encodes an amino acid sequence with a sequence identity of at least 96%, such as 97%, e.g. 98%, such as 99%, e.g. 100% sequence identity to that of SEQ ID NO 1 or a fragment hereof.

In a further aspect, the present invention is directed towards an isolated nucleic acid molecule which encodes a human hepatitis C virus of genotype 4a, strain ED43, wherein said molecule has a nucleic acid sequence with a sequence identity of at least 95% to that of SEQ ID NO: 6 or a fragment hereof and wherein said molecule
comprises the following adaptive mutations: A4211G, A4733G, G5354T, C5697T, A5804G, A5933G, G5949A, A7578G, C7975A, T8365G, A8730G, G8756A, A8757C, C9087T, A9267G, A9273T, A9281G, T9312G and G9315A according to SEQ ID NO: 6;
optionally, further comprises the following adaptive mutation A2819G according to SEQ ID NO: 6; and
one of the following groups of additional adaptive mutations:
   a) A9132G according to SEQ ID NO: 6;
   b) T1152G, T1712C, A2883G, T4736A, T5054C, G6066C, G7109A, A7325G, A8131T, T8718C, A9132G and T9284C according to SEQ ID NO: 6; or
   c) T1152G, T1712C, A2883G, T4736A, T5054C, G6066C, G6257A, G7109A, A7325G, A8131T, T8718C, and T9284C according to SEQ ID NO: 6.

In an embodiment, the nucleic acid molecule which encodes a human hepatitis C virus of genotype 4a, strain ED43, has a nucleic acid sequence with a sequence identity of at least 95% to that of SEQ ID NO: 6 or a fragment hereof and wherein said molecule
comprises the following adaptive mutations: A2819G, A4211G, A4733G, G5354T, C5697T, A5804G, A5933G, G5949A, A7578G, C7975A, T8365G, A8730G, G8756A, A8757C, C9087T, A9267G, A9273T, A9281G, T9312G and G9315A according to SEQ ID NO: 6; and
one of the following groups of additional adaptive mutations:
   a) A9132G according to SEQ ID NO: 6;
   b) T1152G, T1712C, A2883G, T4736A, T5054C, G6066C, G7109A, A7325G, A8131T, T8718C, A9132G and T9284C according to SEQ ID NO: 6; or
   c) T1152G, T1712C, A2883G, T4736A, T5054C, G6066C, G6257A, G7109A, A7325G, A8131T, T8718C, and T9284C according to SEQ ID NO: 6.

In an embodiment, the nucleic acid molecule which encodes a human hepatitis C virus of genotype 4a, strain ED43, has a nucleic acid sequence with a sequence identity of at least 95% to that of SEQ ID NO: 6 or a fragment hereof and comprises the following adaptive mutations: A2819G, A4211G, A4733G, G5354T, C5697T, A5804G, A5933G, G5949A, A7578G, C7975A, T8365G, A8730G, G8756A, A8757C, C9087T, A9132G, A9267G, A9273T, A9281G, T9312G and G9315A according to SEQ ID NO: 6.

In an embodiment, the nucleic acid molecule which encodes a human hepatitis C virus of genotype 4a, strain ED43, has a nucleic acid sequence with a sequence identity of at least 95% to that of SEQ ID NO: 6 or a fragment hereof and comprises the following adaptive mutations: T1152G, T1712C, A2819G, A2883G, A4211G, A4733G, T4736A, T5054C, G5354T, C5697T, A5804G, A5933G, G5949A, G6066C, G7109A, A7325G, A7578G, C7975A, A8131T, T8365G, T8718C, A8730G, G8756A, A8757C, C9087T, A9132G, A9267G, A9273T, A9281G, T9284C, T9312G and G9315A according to SEQ ID NO: 6.

In an embodiment, the nucleic acid molecule which encodes a human hepatitis C virus of genotype 4a, strain ED43, has a nucleic acid sequence with a sequence identity of at least 95% to that of SEQ ID NO: 6 or a fragment hereof and comprises the following adaptive mutations: T1152G, T1712C, A2819G, A2883G, A4211G, A4733G, T4736A, T5054C, G5354T, C5697T, A5804G, A5933G, G5949A, G6066C, G6257A, G7109A, A7325G, A7578G, C7975A, A8131T, T8365G, T8718C, A8730G, G8756A, A8757C, C9087T, A9267G, A9273T, A9281G, T9284C, T9312G and G9315A according to SEQ ID NO: 6.

In a further embodiment, the nucleic acid molecule further comprises the following mutation: G38A according to SEQ ID NO: 6.

In an embodiment, the nucleic acid molecule which encodes a human hepatitis C virus of genotype 4a, strain ED43, has a nucleic acid sequence with a sequence identity of at least 95% to that of SEQ ID NO: 6 or a fragment hereof and comprises the following adaptive mutations: G38A, T1152G, T1712C, A2819G, A2883G, A4211G, A4733G, T4736A, T5054C, G5354T, C5697T, A5804G, A5933G, G5949A, G6066C, G6257A, G7109A, A7325G, A7578G, C7975A, A8131T, T8365G, T8718C, A8730G, G8756A, A8757C, C9087T, A9267G, A9273T, A9281G, T9284C, T9312G and G9315A according to SEQ ID NO: 6.

In a further embodiment, the nucleic acid molecule as described has a nucleic acid sequence with a sequence identity of at least 96%, such as 97%, e.g. 98%, such as 99%, e.g. 100% sequence identity to that of SEQ ID NO 6 or a fragment hereof.

Throughout the description the substitutions as described herein is to be interpreted as for example for A1672S that alanine (A) at amino acid position 1672 is changed to serine(S), L2991R that leucine (L) at the amino acid position 2991 is changed to arginine (R) and so forth.

Thus, A1672S according to SEQ ID NO 1 is to be interpreted that alanine (A) at amino acid position 1672 in SEQ ID NO 1 would be changed to serine(S).

Throughout the description the meaning of the adaptive mutations as described herein is to be interpreted as for example for A9132G that adenine (A) at nucleic acid position 9132 is changed to guanine (G) and so forth.

Thus, A9132G according to SEQ ID NO 6 is to be interpreted that adenine (A) at nucleotide position which would align to nucleotide position 9132 in SEQ ID NO 6 would be changed to guanine (G).

The terms “isolate” and “strain” are used herein interchangeably.

Thus, one aspect of the present invention relates to an isolated nucleic acid molecule which encodes a human hepatitis C virus wherein the hepatitis C virus is derived from genotype 4a.

The present inventors have identified a wide variety of isolates that generated different virus viability.

These isolates are described in the examples of the present application and are disclosed in the sequence listing as SEQ ID NO: 2-5 (amino acid sequences) and SEQ ID NO: 7-10 (nucleic acid sequences).

In an embodiment of the present invention, these sequences are isolated nucleic acid sequences and amino acid sequence, respectively.

In one embodiment, the molecule as described herein is strain ED43cc (SEQ ID NO: 7), strain ED43-31m opt (SEQ ID NO: 8), strain ED43-31m (SEQ ID NO: 9) or strain ED43-20m (SEQ ID NO: 10).

Another aspect of the present invention relates to an isolated nucleic acid molecule being ED43cc (SEQ ID NO: 7).

Another aspect of the present invention relates to an isolated nucleic acid molecule encoding the amino acid sequence according to SEQ ID NO: 2. Another aspect relates to an isolated amino acid molecule ED43cc (SEQ ID NO: 2). In a further embodiment, the nucleic acid molecule encodes an amino acid sequence according to SEQ ID NO: 2 and further comprises an adaptive mutation being G38A according to SEQ ID NO: 6. Another embodiment relates to a nucleic acid molecule encoding an amino acid sequence with a sequence identity of at least 80% to that of SEQ ID NO 2.

In another embodiment, the nucleic acid molecule encoding an amino acid sequence with a sequence sharing at least 85% identity with that set forth in SEQ ID NO: 2, such as 90% identity, 91% identity, 92% identity, 93% identity, 94% identity, 95% identity, 96% identity, 97% identity, 98% identity, or 99% identity.

Another aspect of the present invention relates to an isolated nucleic acid molecule being ED43-31m opt (SEQ ID NO: 8). The strain ED43-31m opt is also named strain ED43-31m/+A1973T/-Q2931R.

Another aspect of the present invention relates to the isolated amino acid molecule ED43-31m/+A1973T/-Q2931R (SEQ ID NO: 3). The strain ED43-31m opt is also named strain ED43-31m/+A1973T/-Q2931R.

Another aspect of the present invention relates to an isolated nucleic acid molecule encoding the amino acid sequence according to SEQ ID NO: 3. Another embodiment relates to a nucleic acid molecule encoding an amino acid sequence with a sequence identity of at least 80% to that of SEQ ID NO: 3.

In another embodiment, the nucleic acid molecule encoding an amino acid sequence with a sequence sharing at least 85% identity with that set forth in SEQ ID NO: 3, such as 90% identity, 91% identity, 92% identity, 93% identity, 94% identity, 95% identity, 96% identity, 97% identity, 98% identity, or 99% identity.

Another aspect of the present invention relates to an isolated nucleic acid molecule being ED43-31m (SEQ ID NO: 9).

Another aspect of the present invention relates to an isolated nucleic acid molecule encoding the amino acid sequence according to SEQ ID NO: 4. Another aspect of the present invention relates to the isolated amino acid molecule ED43-31m (SEQ ID NO: 4).

Another embodiment relates to a nucleic acid molecule encoding an amino acid sequence with a sequence identity of at least 80% to that of SEQ ID NO: 4.

In another embodiment, the nucleic acid molecule encoding an amino acid sequence with a sequence sharing at least 85% identity with that set forth in SEQ ID NO: 4, such as 90% identity, 91% identity, 92% identity, 93% identity, 94% identity, 95% identity, 96% identity, 97% identity, 98% identity, or 99% identity.

Another aspect of the present invention relates to an isolated nucleic acid molecule being ED43-20m (SEQ ID NO: 10).

Another aspect of the present invention relates to an isolated nucleic acid molecule encoding the amino acid sequence according to SEQ ID NO: 5. Another aspect of the present invention relates to the isolated amino acid molecule ED43-20m (SEQ ID NO: 5).

Another embodiment relates to a nucleic acid molecule encoding an amino acid sequence with a sequence identity of at least 80% to that of SEQ ID NO: 5.

In another embodiment, the nucleic acid molecule encoding an amino acid sequence with a sequence sharing at least 85% identity with that set forth in SEQ ID NO: 5, such as 90% identity, 91% identity, 92% identity, 93% identity, 94% identity, 95% identity, 96% identity, 97% identity, 98% identity, or 99% identity.

As commonly defined “identity” is here defined as sequence identity between genes or proteins at the nucleotide or amino acid level, respectively. Thus, in the present context “sequence identity” is a measure of identity between proteins at the amino acid level and a measure of identity between nucleic acid at nucleotide level. The protein sequence identity may be determined by comparing the amino acid sequence in a given position in each sequence when the sequences are aligned. Similarly, the nucleic acid sequence identity may be determined by comparing the nucleotide sequence in a given position in each sequence when the sequences are aligned.

To determine the percent identity of two amino acid sequences or of two nucleic acids, the sequences are aligned for optimal comparison purposes (e.g. gaps may be introduced in the sequence of a first amino or nucleic acid sequence for optimal alignment with a second amino or nucleic acid sequence). The amino acid residues or nucleotides at corresponding amino acid or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=#of identical positions/total #of positions (e.g., overlapping positions)×100).

In one embodiment, the two sequences are the same length.

In another embodiment, the two sequences are of different length and gaps are seen as different positions.

One may manually align the sequences and count the number of identical amino acids. Alternatively, alignment of two sequences for the determination of percent identity may be accomplished using a mathematical algorithm. Such an algorithm is incorporated into the NBLAST and XBLAST programs of (Altschul et al. 1997; Altschul et al. 2005). BLAST nucleotide searches may be performed with the NBLAST program, score=100, wordlength=12, to obtain nucleotide sequences homologous to a nucleic acid molecules of the invention. BLAST protein searches may be performed with the XBLAST program, score=50, wordlength=3 to obtain amino acid sequences homologous to a protein molecule of the invention. To obtain gapped alignments for comparison purposes, Gapped BLAST may be utilised. Alternatively, PSI-Blast may be used to perform an iterated search which detects distant relationships between molecules. When utilising the NBLAST, XBLAST, and Gapped BLAST programs, the default parameters of the respective programs may be used. See http://www.ncbi.nlm.nih.gov. Alternatively, sequence identity may be calculated after the sequences have been aligned e.g. by the BLAST program in the EMBL database (www.ncbi.nlm.gov/cgi-bin/BLAST). Generally, the default settings with respect to e.g. “scoring matrix” and “gap penalty” may be used for alignment. In the context of the present invention, the BLASTN and PSI BLAST default settings may be advantageous.

The percent identity between two sequences may be determined using techniques similar to those described above, with or without allowing gaps. In calculating percent identity, only exact matches are counted.

An embodiment of the present invention thus relates to sequences of the present invention that has some degree of sequence variation.

Several of the sequences of the present invention have been submitted to genbank: ED43 (SEQ ID NOs: 1 and 6) and corresponds to GenBank accession number GU814265. ED43cc (SEQ ID NOs: 2 and 7) and corresponds to GenBank accession number MW531222.

It should be noted that while several of the sequences in the present application (SEQ ID NOS: 6-10) are DNA sequences, the present invention contemplates the corresponding RNA sequence, and DNA and RNA complementary sequences as well. Thus, “nucleic acid molecule” is to be understood as including both DNA and RNA.

Thus, in cases where a DNA sequence is mentioned such DNA sequence refers also to the RNA equivalent i.e. with Ts exchanged with Us as well as their complimentary sequences.

In another embodiment, the HCV nucleic acid is a DNA that codes on expression or after in vitro transcription for a replication-competent HCV RNA genome, or is itself a replication-competent HCV RNA genome.

In one embodiment, the HCV nucleic acid of the invention has a full-length sequence as depicted in or corresponding to the sequences of the present invention.

In another embodiment, the HCV nucleic acid molecule according to the present invention is a fragment of SEQ ID NO: 1 or a fragment having at least 95% sequence identity with SEQ ID NO: 1.

In the present context, “fragment” is to be understood as a part of the encoded amino acid sequence or as part of the nucleic acid sequence i.e. fragments are not full-length sequences. Thus, these amino acid sequences or nucleic acid sequences are shorter than full-length amino acid sequence or nucleic acid sequence, respectively by virtue of truncation of the N-terminus or C-terminus of the amino acid sequence or both or by virtue of deletion of an internal portion or region or more internal portions or regions of the amino acid sequence or nucleic acid sequence. These fragments only comprise some of the structural or non-structural genes or part hereof. The at least 95% sequence identity is to be understood that the fragment would show at least 95% sequence identity to the corresponding fragment of SEQ ID NO: 1. Fragments of an amino acid sequence or a nucleic acid sequence may be generated by methods known in the art.

The different regions of strain ED43 is as follows: Core (1-191), E1 (192-383), E2 (384-746), p7 (747-809), NS2 (810-1026), NS3 (1027-1657), NS4A (1658-1711), NS4B (1712-1972), NS5A (1973-2417), and NS5B (2418-3008), where the numbers in the parentheses indicates amino acids according to SEQ ID NO: 1.

The different regions of strain ED43 is as follows: 5′UTR (1-340), Core (341-913), E1 (914-1489), E2 (1490-2578), p7 (2579-2767), NS2 (2768-3418), NS3 (3419-5311), NS4A (5312-5473), NS4B (5474-6256), NS5A (6257-7591), NS5B (7592-9364), and 3′UTR (9368-9577) where the numbers in the parentheses indicates nucleic acids according to SEQ ID NO: 6.

In one embodiment, the nucleic acid molecule encodes an amino acid sequence corresponding to NS3-NS5B between amino acids 1027-3008 of SEQ ID NO: 1 or an amino acid sequence with a sequence identity of at least 95% to the amino acids 1027-3008 of SEQ ID NO: 1. Thus, the fragment comprises the sequence encoding the genes NS3-NS5B or a part hereof.

In a further embodiment, the nucleic acid molecule encodes an amino acid sequence corresponding to NS3-NS5B between amino acids 1027-3008 of SEQ ID NO: 2 or an amino acid sequence with a sequence identity of at least 95% to the amino acids 1027-3008 of SEQ ID NO: 2. Thus, the fragment comprises the sequence encoding the genes NS3-NS5B or a part hereof.

In a further embodiment, the nucleic acid molecule encodes an amino acid sequence corresponding to NS2-NS5B between amino acids 810-3008 of SEQ ID NO: 1 or an amino acid sequence with a sequence identity of at least 95% to the amino acids 810-3008 of SEQ ID NO: 1. Thus, the fragment comprises the sequence encoding the genes NS2-NS5B or a part hereof.

In a further embodiment, the nucleic acid molecule encodes an amino acid sequence corresponding to NS2-NS5B between amino acids 810-3008 of SEQ ID NO: 2 or an amino acid sequence with a sequence identity of at least 95% to the amino acids 810-3008 of SEQ ID NO: 2. Thus, the fragment comprises the sequence encoding the genes NS2-NS5B or a part hereof.

In a further embodiment, the nucleic acid molecule further comprises the 5′UTR region between nucleic acids 1-340 of SEQ ID NO: 6 or a nucleic acid sequence with a sequence identity of at least 95% to nucleic acids 1-340 of SEQ ID NO: 6.

In a further embodiment, the nucleic acid molecule further comprises the 5′UTR region between nucleic acids 1-340 of SEQ ID NO: 7 or a nucleic acid sequence with a sequence identity of at least 95% to nucleic acids 1-340 of SEQ ID NO: 7.

In an even further embodiment, the nucleic acid molecule further comprises the 3′UTR region between nucleic acids 9368-9577 of SEQ ID NO: 6 or a nucleic acid sequence with a sequence identity of at least 95% to nucleic acids 9368-9577 of SEQ ID NO: 6.

In an even further embodiment, the nucleic acid molecule further comprises the 3′UTR region between nucleic acids 9368-9577 of SEQ ID NO: 7 or a nucleic acid sequence with a sequence identity of at least 95% to nucleic acids 9368-9577 of SEQ ID NO: 7.

Various modifications for example of the 5′ and 3′ UTR are also contemplated by the invention.

In one embodiment, the nucleic acid molecule comprises the sequence encoding the genes NS2-NS5B or part hereof as disclosed above and a 5′UTR region such as a 5′UTR region as disclosed herein. In a further embodiment, the nucleic acid molecule comprises the sequence encoding the genes NS2-NS5B or part hereof as disclosed above and a 3′UTR region such as a 3′UTR region as disclosed herein. In a still further embodiment, the nucleic acid molecule comprises the sequence encoding the genes NS2-NS5B or part hereof as disclosed above, a 3′UTR region and a 5′UTR region such as a 5′UTR region and 3′UTR region as disclosed herein.

In one embodiment, the nucleic acid molecule comprises the sequence encoding the genes NS3-NS5B or part hereof as disclosed above and a 5′UTR region such as a 5′UTR region as disclosed herein. In a further embodiment, the nucleic acid molecule comprises the sequence encoding the genes NS3-NS5B or part hereof as disclosed above and a 3′UTR region such as a 3′UTR region as disclosed herein. In a still further embodiment, the nucleic acid molecule comprises the sequence encoding the genes NS3-NS5B or part hereof as disclosed above, a 3′UTR region and a 5′UTR region such as a 5′UTR region and a 3′UTR region as disclosed herein.

In another embodiment, the nucleic acid further comprises a reporter gene, which, in one embodiment, is a gene encoding neomycin phosphotransferase, Renilla luciferase, secreted alkaline phosphatase (SEAP), Gaussia luciferase or the green fluorescent protein.

Naturally, as noted above, the HCV nucleic acid sequence of the invention is selected from the group consisting of double stranded DNA, positive-sense cDNA, or negative-sense cDNA, or positive-sense RNA or negative-sense RNA or double stranded RNA.

Thus, where particular sequences of nucleic acids of the invention are set forth, both DNA and corresponding RNA are intended, including positive and negative strands thereof.

In a further embodiment, the nucleic acid sequences or the nucleic acid sequences with any mutation described in this document is obtained by any other means than what is described above.

Nucleic acid molecules according to the present invention may be inserted in a plasmid vector for translation of the corresponding HCV RNA. Thus, the HCV DNA may comprise a promoter 5′ of the 5′-UTR on positive-sense DNA, whereby transcription of template DNA from the promoter produces replication-competent RNA. The promoter can be selected from the group consisting of a eukaryotic promoter, yeast promoter, plant promoter, bacterial promoter, or viral promoter.

Thus, in one embodiment the present invention provides a cassette vector for cloning viral genomes, comprising, inserted therein, the nucleic acid sequence according to the invention and having an active promoter upstream thereof.

### Adaptive Mutations

Adapted mutants of a HCV-cDNA construct or HCV-RNA full-length genome with improved abilities to generate infectious viral particles in cell culture compared to the original HCV-cDNA construct or the original HCV-RNA full-length genome are characterized in that they are obtainable by a method in which the type and number of mutations in a cell culture adapted HCV-RNA genome are determined through sequence analysis and sequence comparison and these mutations are introduced into a HCV-cDNA construct, particularly a HCV-cDNA construct according to the present invention, or into an (isolated) HCV-RNA full-length genome, either by site-directed mutagenesis, or by exchange of DNA fragments containing the relevant mutations.

The present inventors here report adaptive mutations, which allow efficient formation and release of viral particles in cell culture, and thus the present invention relates to these adaptive mutations in the present use as well as use in other strains by changing equivalent positions of such genomes to the adapted nucleotide or amino acid described.

A group of preferred HCV-cDNA constructs, HCV-RNA full-length genomes with the ability to release viral particles in cell culture, which are consequently highly suitable for practical use, is characterized in that it contains one, several or all of the nucleic acid exchanges listed below and/or one or several or all of the following amino acid exchanges.

Another group of preferred HCV-cDNA constructs, HCV-RNA replication genomes with the ability to replicate in cell culture, which are consequently highly suitable for practical use, is characterized in that it contains one, several or all of the nucleic acid exchanges listed below and/or one or several or all of the following amino acid exchanges.

One embodiment of the present invention relates to adaptive mutations, wherein the adaptive mutation is a mutation that can be observed by clonal or direct sequencing of recovered replicating genomes of the sequences of the present invention.

Thus in a further embodiment, the present invention relates to nucleic acid molecules according to the present invention, wherein said molecule comprises one or more adaptive mutations in E1, E2, p7, NS2, NS3, NS4A, NS4B, NS5A or NS5B singly or in combination.

In the context of the present invention the term “adaptive mutation” is meant to cover mutations identified in passaged viruses that provide the original and any other HCV sequence the ability to grow efficiently in culture. Furthermore, all introductions of mutations into the sequences described, whether or not yielding better growth abilities, and the introduction of these mutations into any HCV sequence should be considered.

Thus the described mutations enable the HCV-RNA genome (e.g. derived from a HCV-cDNA clone) to form viral particles in and release these from suitable cell lines. In addition some of the described mutations might change the function of the concerned proteins in favourable ways, which might be exploited in other experimental systems employing these proteins.

This also includes other HCV genomes with adaptive mutations, all of them, combinations of them or individual mutations that grow in culture.

It should be understood that any feature and/or aspect discussed above in connection with the mutations according to the invention apply by analogy to both single mutations and any combination of the mutations.

In another embodiment all the amino acid changes observed herein are provided by the present application. The skilled addressee can easily obtain the same amino acid change by mutating another base of the codon and hence all means of obtaining the given amino acid sequence is intended. In one embodiment, the adaptive mutation may be described according to the amino acid sequence and the mutation/change in amino acid observed i.e. the substitution of one amino acid with another.

### Titer

To determine the efficiency of the developed system, HCV RNA titers are determined in IU/ml (international units/ml) with Taq-Man Real-Time-PCR and infectious titers are determined with a focus forming unit assay.

The infectious titers are determined as TCID50/ml (median tissue culture infectious dose/ml) or FFU/ml (focus forming unites/ml); in such method, infectivity titers are determined by infection of cell culture replicates with serial dilutions of virus containing supernatants and, following immuno-stainings for HCV antigens, counting of HCV-antigen positive cell foci.

HCV RNA titers and infectivity titers can be determined extracellularly, in cell culture supernatant (given as IU and TCID₅₀ or FFU per ml, respectively) or intracellularly, in lysates of pelleted cells (given as IU and TCID₅₀ or FFU related to the given cell number or culture plate wells, which was lysed).

In one embodiment, said molecule is capable of generating a HCV infectivity titer of 2 log₁₀ FFU/ml (focus forming unites)/ml or above following transfection and/or subsequent viral passage.

In another embodiment, the present invention relates to a nucleic acid molecule according to the invention, wherein said molecule is capable of generating a HCV infectivity titer of at least 10² FFU/ml or above following transfection and/or subsequent viral passage, such as a titer of at least 10³ FFU/ml, such as a titer of at least 10⁴ FFU/ml, such as a titer of at least 10⁵ FFU/ml.

It is of course evident to the skilled addressee that the titers described here are obtained using the assay described in this text. Any similar or equivalent titer determined by any method is thus evidently within the scope of the present invention.

### Compositions

One embodiment of the present invention relates to a composition comprising a nucleic acid molecule according to the invention suspended in a suitable amount of a pharmaceutical acceptable diluent or excipient.

In another embodiment, this invention provides for compositions comprising an isolated nucleic acid, vector or cell of this invention, or an isolated nucleic acid obtained via the methods of this invention.

In one embodiment, the term “composition” refers to any such composition suitable for administration to a subject, and such compositions may comprise a pharmaceutically acceptable carrier or diluent, for any of the indications or modes of administration as described. The active materials in the compositions of this invention can be administered by any appropriate route, for example, orally, parenterally, intravenously, intradermally, subcutaneously, or topically, in liquid or solid form.

It is to be understood that any applicable drug delivery system may be used with the compositions and/or agents/vectors/cells/nucleic acids of this invention, for administration to a subject, and is to be considered as part of this invention.

The compositions of the invention can be administered as conventional HCV therapeutics. The compositions of the invention may include more than one active ingredient which interrupts or otherwise alters groove formation, or occupancy by RNA or other cellular host factors, in one embodiment, or replicase components, in another embodiment, or zinc incorporation, in another embodiment.

The precise formulations and modes of administration of the compositions of the invention will depend on the nature of the anti-HCV agent, the condition of the subject, and the judgment of the practitioner. Design of such administration and formulation is routine optimization generally carried out without difficulty by the practitioner.

It is to be understood that any of the methods of this invention, whereby a nucleic acid, vector or cell of this invention is used, may also employ a composition comprising the same as herein described, and is to be considered as part of this invention.

“Pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopoeia or other generally recognized pharmacopoeia for use in animals, and more particularly in humans.

The term “excipient” refers to a diluent, adjuvant, carrier, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

The term “adjuvant” refers to a compound or mixture that enhances the immune response to an antigen. An adjuvant can serve as a tissue depot that slowly releases the antigen and also as a lymphoid system activator that non-specifically enhances the immune response. Often, a primary challenge with an antigen alone, in the absence of an adjuvant, will fail to elicit a humoral or cellular immune response.

Adjuvants include, but are not limited to, complete Freund's adjuvant, incomplete Freund's adjuvant, saponin, mineral gels such as aluminum hydroxide, surface active substances such as lysolecithin, pluronicpolyols, polyanions, peptides, oil or hydrocarbon emulsions, keyhole limpet hemocyanins, dinitrophenol, and potentially useful human adjuvants such as BCG (bacilleCalmette-Guerin) and Corynebacteriumparvmm.

Preferably, the adjuvant is pharmaceutically acceptable.

Thus, one embodiment of the present invention relates to a composition comprising a nucleic acid molecule according to the present invention suspended in a suitable amount of a pharmaceutical acceptable diluent or excipient.

### Cells

The nucleotides of the present invention may be used to provide a method for identifying additional cell lines that are permissive for infection with HCV, comprising contacting (e.g. transfecting) a cell line in tissue culture with an infectious amount of HCV RNA of the present invention, e.g., as produced from the plasmid clones, and detecting replication and/or formation and release of viral particles of HCV in cells of the cell line.

Naturally, the invention extends as well to a method for identifying an animal that is permissive for infection with HCV, comprising introducing an infectious amount of the HCV RNA, e.g., as produced by the plasmids, to the animal, and detecting replication and/or formation and release of viral particles of HCV in the animal. By providing infectious HCV, e.g. comprising a dominant selectable marker, the invention further provides a method for selecting for HCV with further adaptive mutations that permit higher levels of HCV replication in a permissive cell line or animal comprising contacting (e.g. transfecting) a cell line in culture, or introducing into an animal, an infectious amount of the HCV RNA, and detecting progressively increasing levels of HCV RNA and infectious HCV viral particles in the cell line or the animal.

In a specific embodiment, the adaptive mutation permits modification of HCV tropism. An immediate implication of this aspect of the invention is creation of new valid cell culture and animal models for HCV infection.

The permissive cell lines or animals that are identified using the nucleic acids of the invention are very useful, inter alia, for studying the natural history of HCV infection, isolating functional components of HCV, and for sensitive, fast diagnostic applications, in addition to producing authentic HCV virus or components thereof.

Because the HCV DNA, e.g., plasmid vectors, of the invention encode HCV components, expression of such vectors in a host cell line transfected, transformed, or transduced with the HCV DNA can be effected.

For example, a baculovirus or plant expression system can be used to express HCV virus particles or components thereof. Thus, a host cell line may be selected from the group consisting of a bacterial cell, a yeast cell, a plant cell, an insect cell, and a mammalian cell.

In one embodiment, the cell is a hepatocyte, or in another embodiment, the cell is the Huh-7 hepatoma cell line or a derived cell line such as Huh7.5 or Huh7.5.1 cell line.

In one embodiment, the cell, or in another embodiment, cell systems of this invention comprise primary cultures or other, also non hepatic cell lines. “Primary cultures” refers, in one embodiment, to a culture of cells that is directly derived from cells or tissues from an individual, as well as cells derived by passage from these cells, or immortalized cells.

In one embodiment, “cell line” refers to a population of cells capable of continuous or prolonged growth and division in vitro. The term “cell lines” also includes immortalized cells. Often, cell lines are clonal populations derived from a single progenitor cell. Such cell lines are also termed “cell clones”. It is further known in the art that spontaneous or induced changes can occur in karyotype during storage or transfer of such clonal populations. Therefore, cells derived from the cell clones referred to may not be precisely identical to the ancestral cells or cultures. According to the present invention, such cell clones may be capable of supporting replication of a vector, virus, viral particle, etc., of this invention, without a significant decrease in their growth properties, and are to be considered as part of this invention.

It is to be understood that any cell of any organism that is susceptible to infection by or propagation of an HCV construct, virus or viral particle of this invention is to be considered as part of this invention, and may be used in any method of this invention, such as for screening or other assays, as described herein.

Thus, one embodiment of the present invention relates to a cell comprising the nucleic acid according to the present invention, the composition of present invention or the cassette vector of the present invention.

Another embodiment of the present invention relates to a method for producing a cell, which replicates human hepatitis C virus and optionally produces a virus particle comprising introducing a nucleic acid molecule of the present invention into a cell. In a further embodiment, the present invention relates to a method for producing a cell, which replicates human hepatitis C virus and produces a virus particle comprising introducing a nucleic acid molecule of the present invention into a cell.

In a preferred embodiment is the cell is a Huh7.5 cell.

Another embodiment of the present invention relates to a cell obtainable by the methods of the present invention.

Also, a method for in vitro producing a hepatitis C virus-infected cell is described comprising culturing the cell which produces virus particles of the present invention and infecting other cells with the produced virus particle in the culture. Another embodiment relates to a method for in vitro producing a hepatitis C virus-infected cell is described comprising culturing a cell and infecting other cells with the produced virus particle in the culture.

Naturally, the invention extends to any cell obtainable by such methods, for example any in vitro cell line infected with HCV, wherein the HCV has a genomic RNA sequence as described herein such as a hepatitis C virus infected cell obtainable by any of the methods described.

In one embodiment, the cell line is a hepatocyte cell line such as Huh7 or derived cell lines e.g. Huh7.5 or Huh7.5.1. In another embodiment, the cell is Huh7.5.

In another embodiment the cell is any cell expressing the genes necessary for HCV infection and replication, such as but not limited to CD81, SR-BI, Claudin-1, -4, -6 or -9, Occludin, and the low-density lipid receptor.

The invention further provides various methods for producing HCV virus particles, including by isolating HCV virus particles from the HCV-infected non-human animal of invention; culturing a cell line of the invention under conditions that permit HCV replication and virus particle formation; or culturing a host expression cell line transfected with HCV DNA under conditions that permit expression of HCV particle proteins; and isolating HCV particles or particle proteins from the cell culture. The present invention extends to an HCV virus particle comprising a replication-competent HCV genome RNA, or a replication-defective HCV genome RNA, corresponding to an HCV nucleic acid of the invention as well.

A further aspect of the present invention relates to a method for producing a hepatitis C virus replication system, comprising culturing a cell according to the present invention to allow the cell to replicate the virus genome.

HCV replication systems using sub-genomic or full-length genomes have been valuable and useful tools for development and preclinical testing of drugs targeting HCV replication. These models provide fundamental tools for testing of drug efficacy in the context of viral replication, and the infectious genotype 4a genomes developed here can be applied to develop such systems.

In one embodiment, the cell being cultured for the replication system only comprises a fragment of the amino acid sequence allowing the HCV to replicate but not to form viruses.

### Virus Particle

The production of authentic virus proteins (antigens) may be used for the development and/or evaluation of diagnostics. The cell culture system according to the invention also allows the expression of HCV antigens in cell cultures. In principle these antigens can be used as the basis for diagnostic detection methods.

The production of HCV viruses and virus-like particles, in particular for the development or production of therapeutics and vaccines as well as for diagnostic purposes is an embodiment of the present invention. Especially cell culture adapted complete HCV genomes, which could be produced by using the cell culture system according to the invention, are able to replicate and form viral particles in cell culture with high efficiency. These genomes have the complete functions of HCV and in consequence they are able to produce infectious viruses.

Thus in one embodiment the present invention relates to a method for producing a hepatitis C virus particle of the present invention or parts thereof, comprising culturing a cell or an animal to allow either to produce the virus. In a further embodiment, the present invention relates to a method for producing a hepatitis C virus particle of the present invention comprising culturing a cell to allow to produce the virus.

In another embodiment, the invention provides a hepatitis C virus particle obtainable by the method described.

In an even further embodiment, the invention relates to a method for producing a hepatitis C virus particle, comprising culturing a cell as described herein to allow the cell to produce the virus.

Because the invention provides, inter alia, infectious HCV RNA, the invention provides a method for infecting an animal with HCV, which comprises administering an infectious dose of HCV RNA, such as the HCV RNA transcribed from the plasmids described above, to the animal. Naturally, the invention provides a non-human animal infected with HCV of the invention, which non-human animal can be prepared by the foregoing methods.

In one embodiment the introduced mutations attenuate the virus in vivo.

A further advantage of the present invention is that, by providing a complete functional HCV genome, authentic HCV viral particles or components thereof, which may be produced with native HCV proteins or RNA in a way that is not possible in subunit expression systems, can be prepared.

In addition, since each component of HCV of the invention is functional (thus yielding the authentic HCV), any specific HCV component is an authentic component, i.e., lacking any errors that may, at least in part, affect the clones of the prior art. Indeed, a further advantage of the invention is the ability to generate HCV virus particles or virus particle proteins that are structurally identical to or closely related to natural HCV virions or proteins. Thus, in a further embodiment, the invention provides a method for propagating HCV in vitro comprising culturing a cell line contacted with an infectious amount of HCV RNA of the invention, e.g., HCV RNA translated from the plasmids described above, under conditions that permit replication of the HCV RNA.

In one embodiment, the method further comprises isolating infectious HCV. In another embodiment, the method further comprises freezing aliquots of said infectious HCV.

According to this aspect of the invention, and in one embodiment, the HCV is infectious following thawing of said aliquots, and in another embodiment, the HCV is infectious following repeated freeze-thaw cycles of said aliquots.

A further embodiment of the present invention relates to a method for in vitro producing a hepatitis C virus-infected cell comprising culturing a cell according to the present invention and infecting other cells with the produced virus particle in the culture.

### Screening for Anti-Viral Drugs and the Determination of Drug Resistance

It can be assumed that resistance to therapy occurs due to the high mutation rate of the HCV genome. This resistance, which is very important for the clinical approval of a substance, can be detected with the cell culture system according to the invention. Cell lines, in which the HCV-RNA construct or the HCV genome or subgenome replicates and produces infectious viral particles, are incubated with increasing concentrations of the relevant substance and the replication of the viral RNA is either determined by means of an introduced reporter gene or through the qualitative or quantitative detection of the viral nucleic acids or proteins. The release of viral particles is determined by measuring HCV RNA and infectivity titers in the cell culture supernatant. Alternatively, the number of antigen-expressing cells is determined. Resistance is given if no or a reduced inhibition of the replication and release of viral particles can be observed with the normal concentration of the active substance. The nucleotide and amino acid replacements responsible for the therapy resistance can be determined by recloning the HCV-RNA (for example by the means of RT-PCR) and sequence analysis. By cloning the relevant replacement(s) into the original construct its causality for the resistance to therapy can be proven.

The systems developed in this invention are ideal candidates for specific testing of therapeutics in general and therapeutics targeting viral entry, assembly and release.

Genomes with the sequences of the present invention are valuable for testing of neutralizing antibodies and other drugs acting on entry level, such as fusion inhibitors.

In one embodiment the present invention relates to a method for identifying neutralizing antibodies.

In another one embodiment the present invention relates to a method for identifying cross-genotype neutralizing antibodies.

In one embodiment the present invention relates to a method of raising neutralizing antibodies.

In another embodiment the present invention relates to a method of raising cross neutralizing antibodies.

In one embodiment the present invention related to a method for screening new HCV genotype 4 inhibitors or neutralizing antibodies, comprising
a) culturing at least one selected from the group consisting of a cell according to the present invention, a hepatitis C virus infected cell according to the present invention and a hepatitis C virus particle obtainable by the present invention together with a hepatitis C virus permissive cell, and
b) subjecting said virus or virus infected cell culture to a blood sample or derivatives thereof from a HCV genotype 4 infected patient
c) detecting the amount of replicating RNA and/or the virus particles.

Inhibitors targeting the HCV non-structural proteins NS3/4A, NS5A and NS5B have been developed, and clinical studies show promising results for these inhibitors. The present invention offers novel culture systems where additional HCV isolates can be tested to generate efficient cross-reactive inhibitors.

The p7 peptide features two transmembrane domains (TM1 and TM2), and p7 monomers multimerize to form a putative ion channel. Additionally p7 has been shown to contain genotype specific sequences required for genotype specific interactions between p7 and other HCV proteins. Hence, new compounds targeting the putative p7 ion-channel and autoprotease inhibitors interfering with NS2, or drugs targeting the viral NS3 helicase region, and drugs targeting cellular proteins involved in the described processes can be tested.

Thus, one embodiment of the present invention relates to a method for screening an anti-hepatitis C virus substance, comprising
a) culturing at least one selected from the group consisting of a cell according to the present invention, a hepatitis C virus infected cell according to the present invention and a hepatitis C virus particle obtainable by the present invention together with a hepatitis C virus permissive cell,
b) subjecting said virus or virus infected cell culture to the anti-hepatitis C virus substance, and
c) detecting the replicating RNA and/or the virus particles in the resulting culture.

Another embodiment of the present invention relates to a method for screening an anti-hepatitis C virus substance, comprising
a) culturing at least one selected from the group consisting of a cell comprising a nucleic acid molecule according to the present invention, a cell as described herein, a hepatitis C virus particle obtainable from a method as described herein and a hepatitis C virus replication obtainable from a method as described herein together with a hepatitis C virus permissive cell, and
b) detecting the replicating RNA or the virus particles in the resulting culture.

Yet another embodiment of the present invention relates to a hepatitis C vaccine comprising a hepatitis C virus particle of the present invention or a part thereof.

In another embodiment, the inhibition of HCV replication and/or infection and/or pathogenesis includes inhibition of downstream effects of HCV. In one embodiment, downstream effects include neoplastic disease, including, in one embodiment, the development of hepatocellular carcinoma.

In one embodiment, the invention provides a method of screening for anti-HCV therapeutics, the method comprising contacting a cell with an isolated nucleic acid molecule encoding an infectious recombinant HCV genome, comprising a chimeric HCV genome or a replicating subunit and contacting the cell with a candidate molecule, independently contacting the cell with a placebo and determining the effects of the candidate molecule on HCV infection, replication, or cell-to-cell spread, versus the effects of the placebo, wherein a decrease in the level of HCV infection, replication, or cell-to-cell spread indicates the candidate molecule is an anti-HCV therapeutic.

In one embodiment, the method may be conducted in vitro or in vivo. In one embodiment, the cells as described may be in an animal model, or a human subject, entered in a clinical trial to evaluate the efficacy of a candidate molecule. In one embodiment, the molecule is labelled for easier detection, including radio-labelled, antibody labelled for fluorescently labelled molecules, which may be detected by any means well known to one skilled in the art.

In one embodiment, the candidate molecule is an antibody.

Another embodiment of the present invention relates to an antibody against the hepatitis C virus particle of the present invention.

In one embodiment, the term “antibody” refers to intact molecules as well as functional fragments thereof, such as Fab, F(ab′)2, and Fv. In one embodiment, the term “Fab” refers to a fragment, which contains a monovalent antigen-binding fragment of an antibody molecule, and in one embodiment, can be produced by digestion of whole antibody with the enzyme papain to yield an intact light chain and a portion of one heavy chain, or in another embodiment can be obtained by treating whole antibody with pepsin, followed by reduction, to yield an intact light chain and a portion of the heavy chain. In one embodiment, the term “F(ab′)2”, refers to the fragment of the antibody that can be obtained by treating whole antibody with the enzyme pepsin without subsequent reduction, F(ab′)2 is a dimer of two Fab′ fragments held together by two disulfide bonds. In another embodiment, the term “Fv” refers to a genetically engineered fragment containing the variable region of the light chain and the variable region of the heavy chain expressed as two chains, and in another embodiment, the term “single chain antibody” or “SCA” refers to a genetically engineered molecule containing the variable region of the light chain and the variable region of the heavy chain, linked by a suitable polypeptide linker as a genetically fused single chain molecule. Also included are chimeric antibodies, for example, monoclonal antibodies or fragments thereof. Further included are camelid antibodies or nanobodies. Methods of producing these functional fragments, chimeric antibodies, camelid antibodies or nanobodies are known in the art.

In another embodiment, the candidate molecule is a small molecule. In one embodiment, the phrase “small molecule” refers to, inter-alia, synthetic organic structures typical of pharmaceuticals, peptides, nucleic acids, peptide nucleic acids, carbohydrates, lipids, and others, as will be appreciated by one skilled in the art. In another embodiment, small molecules, may refer to chemically synthesized peptidomimetics of the 6-mer to 9-mer peptides of the invention.

In another embodiment, the candidate molecule is a nucleic acid. Numerous nucleic acid molecules can be envisioned for use in such applications, including antisense, siRNA, ribozymes, etc., as will be appreciated by one skilled in the art.

It is to be understood that the candidate molecule identified and/or evaluated by the methods of this invention, may be any compound, including, inter-alia, a crystal, protein, peptide or nucleic acid, and may comprise an HCV viral product or derivative thereof, of a cellular product or derivative thereof. The candidate molecule in other embodiments may be isolated, generated synthetically, obtained via translation of sequences subjected to any mutagenesis technique, or obtained via protein evolution techniques, well known to those skilled in the art, each of which represents an embodiment of this invention, and may be used in the methods of this invention, as well.

In one embodiment, the compound identified in the screening methods as described, may be identified by computer modelling techniques, and others, as described herein. Verification of the activity of these compounds may be accomplished by the methods described herein, where, in one embodiment, the test compound demonstrably affects HCV infection, replication and/or pathogenesis in an assay, as described. In one embodiment, the assay is a cell-based assay, which, in one embodiment, makes use of primary isolates, or in another embodiment, cell lines, etc. In one embodiment, the cell is within a homogenate, or in another embodiment, a tissue slice, or in another embodiment, an organ culture. In one embodiment, the cell or tissue is hepatic in origin, or is a derivative thereof. In another embodiment, the cell is a commonly used mammalian cell line, which has been engineered to express key molecules known to be, or in another embodiment, thought to be involved in HCV infection, replication and/or pathogenesis.

In another embodiment, protein, or in another embodiment, peptide or in another embodiment, other inhibitors of the present invention cause inhibition of infection, replication, or pathogenesis of HCV in vitro or, in another embodiment, in vivo when introduced into a host cell containing the virus, and may exhibit, in another embodiment, an EC50 in the range of from about 0.0001 nM to 100 μM in an in vitro assay for at least one step in infection, replication, or pathogenesis of HCV, more preferably from about 0.0001 nM to 75 μM, more preferably from about 0.0001 nM to 50 μM, more preferably from about 0.0001 nM to 25 μM, more preferably from about 0.0001 nM to 10 μM, and even more preferably from about 0.0001 nM to 1 μM.

In another embodiment, the inhibitors of HCV infection, or in another embodiment, replication, or in another embodiment, pathogenesis, may be used, in another embodiment, in ex vivo scenarios, such as, for example, in routine treatment of blood products wherein a possibility of HCV infection exists, when serology shows a lack of HCV infection.

In another embodiment, the anti-HCV therapeutic compounds identified via any of the methods of the present invention can be further characterized using secondary screens in cell cultures and/or susceptible animal models. In one embodiment, a small animal model may be used, such as, for example, urokinase-type plasminogen activator-severe combined immunodeficiency (uPA-SCID) mice with human liver xenografts (human liver chimeric mice) or a tree shrew Tupaia belangeri chinensis. In another embodiment, an animal model may make use of a chimpanzee. Test animals may be treated with the candidate compounds that produced the strongest inhibitory effects in any of the assays/methods of this invention. In another embodiment, the animal models provide a platform for pharmacokinetic and toxicology studies.

### Vaccines

The construct according to the invention by itself can also be used for various purposes in all its embodiments. This includes the construction of hepatitis C viruses or HCV-like particles and their production in cell cultures as described.

HCV or HCV-like particles, as well as deduced peptides or expressed recombinant proteins, can be used in particular as vaccine. Thus, one embodiment of the present invention relates to a hepatitis C vaccine comprising a hepatitis C virus particle according to the invention or a part thereof.

In another embodiment, the nucleic acids, vectors, viruses, or viral particles may be further engineered to express a heterologous protein, which, in another embodiment, is mammalian or a derivative thereof, which is useful in combating HCV infection or disease progression. Such proteins may comprise cytokines, growth factors, tumor suppressors, or in one embodiment, may following infection, be expressed predominantly or exclusively on an infected cell surface. According to this aspect of the invention, and in one embodiment, such molecules may include costimulatory molecules, which may serve to enhance immune response to infected cells, or preneoplastic cells, or neoplastic cells, which may have become preneoplastic or neoplastic as a result of HCV infection. In one embodiment, the heterologous sequence encoded in the nucleic acids, vectors, viruses, or viral particles of this invention may be involved in enhanced uptake of a nucleic acids, vectors, viruses, or viral particles, and may specifically target receptors thought to mediate HCV infection.

Further, the present invention relates to a method for producing a hepatitis C virus vaccine comprising using a hepatitis C virus particle according to the invention as an antigen, and naturally any antibody against such hepatitis C virus particle.

### Uses

The cell culture system developed of the present invention will be a valuable tool to address different research topics.

It will allow the isolate, subtype and genotype specific study of functions of all HCV genome regions and proteins using reverse genetics.

Accordingly, the developed cell culture systems allow individual patient targeting. This means that when a new potential therapeutic candidate is discovered it is possible to test this particular candidate or combination of candidates on novel HCV isolates grown in culture.

Knowing which specific genotype the candidate is functioning towards, it allows an individual treatment of each patient dependent on which specific genotype the patient is infected with. Furthermore, these cell culture systems allow the development of antibodies and vaccines targeting individual patients.

The replication level of a virus can be determined, in other embodiments, using techniques known in the art, and in other embodiments, as exemplified herein. For example, the genome level can be determined using RT-PCR, and northern blot. To determine the level of a viral protein, one can use techniques including ELISA, immunoprecipitation, immunofluorescence, EIA, RIA, and Western blotting analysis.

In one embodiment, the invention provides a method of identifying sequences in HCV associated with HCV pathogenicity, comprising contacting cells with an isolated nucleic acid molecule encoding an infectious recombinant HCV genome, contacting cells with an isolated nucleic acid molecule comprising at least one mutation, independently culturing the cells and determining HCV infection, replication, or cell-to-cell spread, in cells contacted with the mutant, versus the recombinant HCV, whereby changes in HCV infection, replication, or cell-to-cell spread in cells contacted with the mutant virus shows the mutation is in an HCV sequence associated with HCV pathogenicity.

In one embodiment, the invention provides a method of identifying HCV variants with improved growth in cell culture, the method comprising contacting cells with an isolated nucleic acid molecule encoding an infectious recombinant HCV genome contacting cells with an isolated nucleic acid molecule comprising at least one mutation, independently culturing the cells and determining HCV infection, replication, or cell-to-cell spread, in cells contacted with the recombinant HCV or the mutated virus, whereby enhanced HCV infection, replication, or cell-to-cell spread in cells contacted with the mutated virus shows that the HCV variant has improved growth in cell culture.

In some embodiments, HCV variants are selected for enhanced replication, over a long course of time, in vitro culture systems. According to this aspect of the invention, and in some embodiments, cells contacted with the variants are characterized by reduced infection, as compared to cells contacted with the recombinant HCV.

### Kits

In a related aspect, the invention also provides a test kit for HCV comprising HCV virus components, and a diagnostic test kit for HCV comprising components derived from an HCV virus as described herein.

Furthermore, the invention also provides test kits, for screening for new HCV inhibitors, neutralizing and cross neutralizing antibodies, comprising HCV virus components.

A further aspect of the present invention relates to a method for obtaining an isolated nucleic acid molecule encoding a human hepatitis C virus with adaptive mutations, comprising identification of one or more adaptive mutations as described in the above method, incorporation of said one or more adaptive mutations into a nucleic acid molecule encoding a full length human hepatitis C virus or a fragment hereof, and isolating the nucleic acid molecule encoding a human hepatitis C virus with adaptive mutations.

One embodiment of the present invention relates to an isolated nucleic acid molecule obtained from the above method.

Another embodiment of the present invention relates to an isolated nucleic acid molecule according to the present invention, wherein the human hepatitis C virus is of genotype 4.

### General

Reference to any prior art in this specification is not, and should not be taken as, an acknowledgment or any form of suggestion that this prior art forms part of the common general knowledge in any country.

All patent and non-patent references cited in the present application, are hereby incorporated by reference in their entirety.

As will be apparent, preferred features and characteristics of one aspect of the invention may be applicable to other aspects of the invention. The invention may be embodied in other specific forms without departing from the spirit or essential characteristics thereof. The foregoing embodiments are therefore to be considered in all respects illustrative rather than limiting on the invention described herein. Scope of the invention is thus showed be the appended claims rather than by the foregoing description, and all changes that come within the meaning and range of equivalency of the claims are intended to be embraced by reference therein.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In addition, singular reference does not exclude a plurality. Thus, references to “a”, “an”, “first”, “second” etc. do not preclude a plurality.

It should be noted that embodiments and features described in the context of one of the aspects of the present invention also apply to the other aspects of the invention.

The invention will hereinafter be described by way of the following non-limiting Figures and Examples.

### Sequences

| SEQ ID | | AA/ | |
|---|---|---|---|
| NO: | Name | NA | GenBank |
| 1 | ED43 | AA | GU814265 |
| 2 | ED43cc | AA | MW531222 |
| 3 | ED43-31m opt | AA | |
| 4 | ED43-31m | AA | |
| 5 | ED43-20m | AA | |
| 6 | ED43 | NA | GU814265 |
| 7 | ED43cc | NA | MW531222 |
| 8 | ED43-31m opt | NA | |
| 9 | ED43-31m | NA | |
| 10 | ED43-20m | NA | |
| 11 | CTAAGGTCGGAGTGTTAAGC | NA | |
| 12 | TGCCTGATAGGGTGCTTGCG | NA | |
| 13 | AGGTCGGAGTGTTAAGCTGCC | NA | |

### EXAMPLES

### Materials and methods

### Construction of HCV Genotype 4a Clones

The in vivo infectious strain ED43 clone was previously described (Gottwein et al. 2010). The chimeric genome comprising ED43 Core-NS5A (C5A) and JFH1-NS5B and-UTRs was generated by replacing the 5′UTR from ED43 5′UTR-NS5A recombinant (Li et al. 2014) with the corresponding JFH1-sequence. Mutations were introduced by QuikChange site-directed mutagenesis kit (Agilent) or by fusion PCR. The HCV sequences of final plasmid preparations were confirmed by Sanger sequencing (Macrogen). The nucleotide (nt) and amino acid (aa) numbers refer to the ED43 full-length recombinant sequence.

### Production and Analysis of Culture Viruses

Viability of HCV recombinants was tested by transfection of RNA-transcripts into Huh7.5 cells using Lipofectamine 2000 (ThermoFisher) (Li et al. 2012b). Cells were sub-cultured every 2-3 days and viral passage was performed as described (Pham et al. 2018). Harvested cellular pellets were centrifuged at 2000 rpm for 5 minutes, washed 2-3 times with sterile PBS (Sigma-Aldrich) and stored in 1 ml of Trizol (ThermoFisher). Infectivity titers were determined in triplicate and reported as log₁₀ focus-forming units (log₁₀ FFU/mL) (Li et al. 2012a).

### Genome Analysis of Recovered Viruses

Next-generation sequencing (NGS) was performed as described (Pham et al. 2018; Jensen et al. 2019; Pham et al. 2019; Fahnøe et al. 2019). Briefly, RNA was extracted, and reverse transcription (RT)-PCR performed to obtain complete HCV open reading frame (ORF) amplicons (Fahnøe et al. 2019). For RT, primer 5′-CTAAGGTCGGAGTGTTAAGC-3′, (SEQ ID NO: 11) and for PCR, primers 5′-TGCCTGATAGGGTGCTTGCG-3′ (SEQ ID NO: 12) and 5′-AGGTCGGAGTGTTAAGCTGCC-3′ (SEQ ID NO: 13) were used. PCR amplicons were processed with NEBNext Ultra II FS DNA Library Prep Kit (New England Biolabs). In order to sequence up to 500 bp to cover NS3P, NS5A domain I or the NS5B-palm domain (up to 167 aa), we performed size-selection. Sequencing was carried out in-house by Illumina Miseq using 500 cycles v2 kit. Data were analyzed for single-nucleotide polymorphism (SNP) (Jensen et al. 2019; Pham et al. 2019). The linkage analysis was done with LinkGE on coding SNPs with frequencies ≥2% (Jensen et al. 2019; Pham et al. 2019). The haplotypes were reconstructed and plotted using GraphPad Prism 6.

For ORF analysis, the linkage and haplotype reconstruction could not be applied in one read pair. Therefore, the frequency development of SNP variants over time was used. For specific samples, PCR amplicons were sub-cloned into TOPO-XL2 vector (ThemoFisher), allowing ORF linkage analyses. Each clone was sequenced by Sanger and aligned to build phylogeny, and ancestral reconstruction (Jensen et al. 2019).

For ED43 (C5A) recombinants, recovered viruses were analyzed by Sanger (Pham et al. 2018). For viral 5′UTR sequences, we used a 5′RACE procedure on culture supernatants (Pham et al. 2018; Li et al. 2012b). The PCR products were analyzed by Sanger (Pham et al. 2018).

### Virus Stocks and Treatment Assays

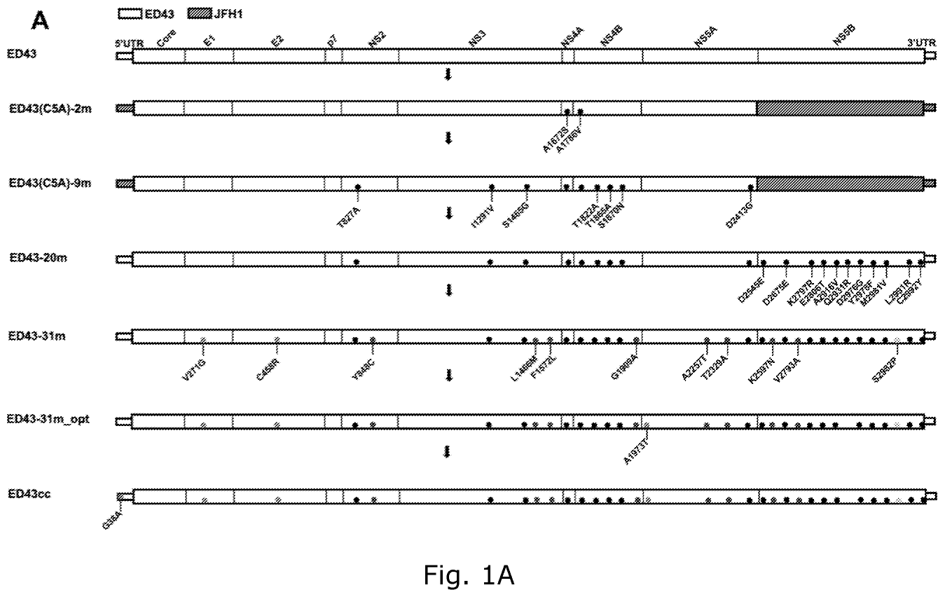

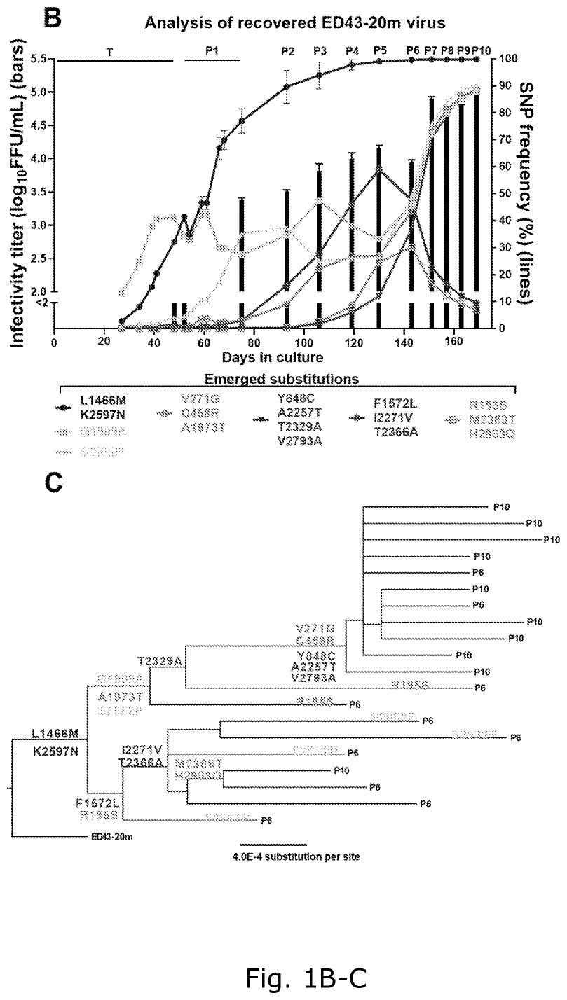

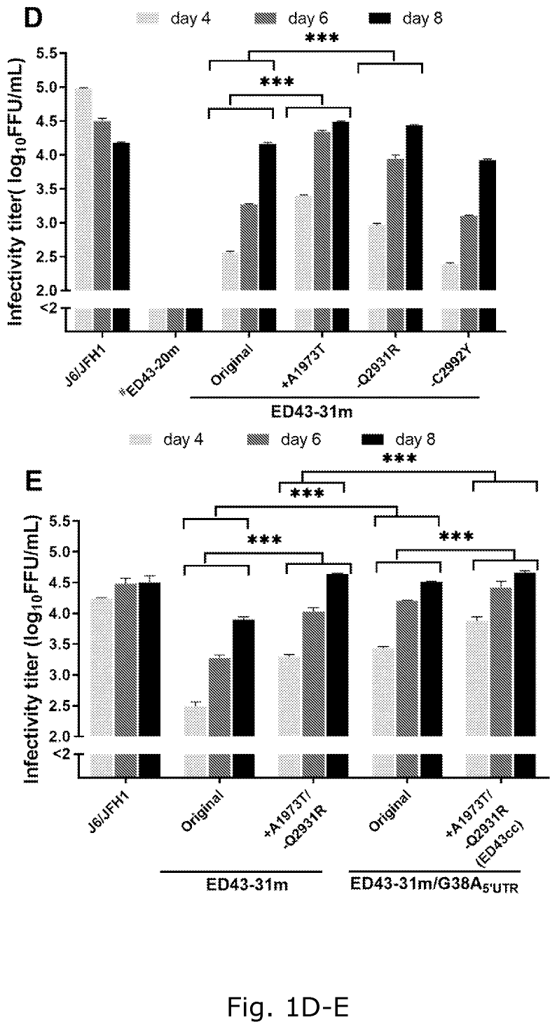

Inhibitors (Acme Bioscience) were diluted in dimethyl sulfoxide (Pham et al. 2018; Ramirez et al. 2016; Ramirez et al. 2014; Gottwein et al. 2018). Escape and treatment assays were conducted using the fifth-, seventh-, and tenth-passages of ED43-20m (FIG. 1A,B), and not the final ED43cc virus, in order to optimize the timing of the different experiments. For the reverse-genetics testing of RAS, the ED43−31m/+A1973T/−Q2931R (named ED43-31m_opt) recombinant was used (FIG. 1A).

Treatments were initiated upon virus spread (≥90% HCV-antigen positive cells) and the inhibitors were added every 2-3 days when cells were sub-cultured (Pham et al. 2018). For concentration-response assays using described methods (Pham et al. 2018; Ramirez et al. 2016; Ramirez et al. 2014), stocks of non-treated and of single-treatment escape viruses were prepared. To prepare virus stocks from escape viruses, supernatants recovered from the treatment experiments were used to infect naïve Huh7.5 cells, which were then cultured without inhibitors until viruses spread. Sequences of virus stocks were confirmed by NGS. The half-maximal effective concentration (EC₅₀) value was calculated using GraphPad Prism 6.

For combination treatments with indicated DAA concentrations, escape stock viruses were used to infect naïve Huh7.5 cells and followed until virus spread (Pham et al. 2018). Viral supernatants were collected at treatment initiation (day 0), and HCV sequences were confirmed by NGS. Unless otherwise stated, the viruses were treated 28-30 days. Afterwards, cultures were followed without drugs for 14 days (Pham et al. 2018); infection was defined as eradicated, if no HCV-antigen positive cells were detected (Pham et al. 2018; Ramirez et al. 2016).

### Results

### Development of HCV Genotype 4a Full-Length Infectious Cell-Culture Systems

Like most prototype HCV clones, the full-length clone of genotype 4a prototype strain ED43 (pED43) is infectious in chimpanzees, but not in Huh7.5 cells (Gottwein et al. 2010). Therefore, we aimed at identifying adaptive substitutions permitting culture of this full-length clone. It has been reported that the JFH1-NS5B has high replication activity in cell culture. Taking advantage of this, we showed that for various genotypes, cell culture adaptation of recombinants with genotype specific Core-NS5A (C5A), and JFH1-NS5B and-UTRs, resulted in identification of mutations that permitted replication of the corresponding full-length genome (Ramirez et al. 2018). Thus, we first generated JFH1-based ED43-C5A recombinants for this same purpose.

### Culture-Efficient ED43 (C5A)-Adapted Recombinant

RNA transcripts from ED43 (C5A)-clones with or without A1672S (NS4A), required for culture of genotype 1a, 2a, and 2b strains (Li et al. 2012a; Li et al. 2015; Li et al. 2012b; Ramirez 2014), yielded no HCV-antigen positive Huh7.5-cells during 30 days follow-up. However, ED43(C5A)-2m (FIG. 1A) with A1786V (NS4B), previously used for adaptation of the ED43 5′UTR-NS5A recombinant (Li et al. 2014), and A1672S(NS4A) spread at day 52 (4.0 log₁₀ FFU/mL) and acquired 5 additional substitutions (Table 3), which were added into ED43(C5A)-2m. The resulting ED43(C5A)-7m spread at day 11 post-transfection with 4.1 log₁₀ FFU/mL in 2^{nd} passage. Adding 2 previously identified substitutions (Pham et al. 2018; Ramirez et al. 2016; Li et al. 2015), led to ED43(C5A)-9m (FIG. 1A, Table 3) spreading at day 6 post-transfection, producing 4.2 log₁₀ FFU/mL in 2^{nd} passage (Table 3).

### Development of High Titer Culture-Infectious-ED43 Full-Length Recombinants

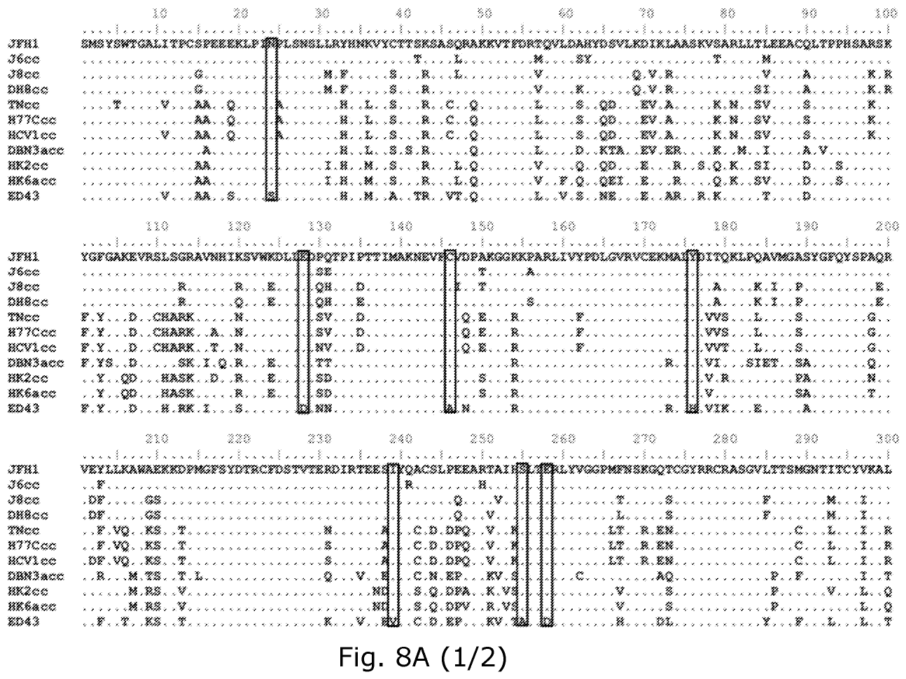

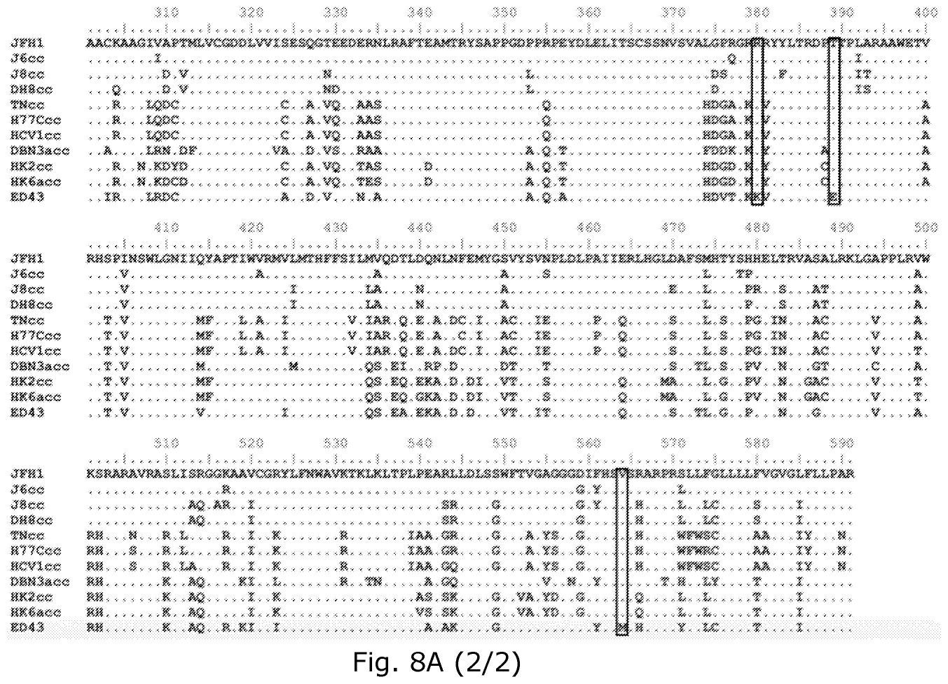

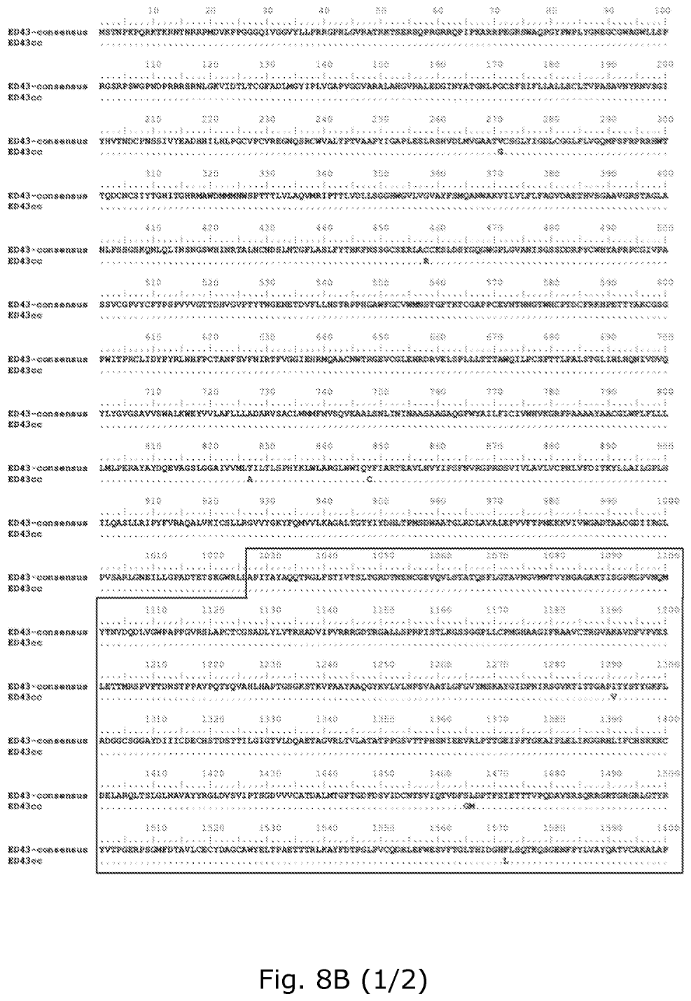

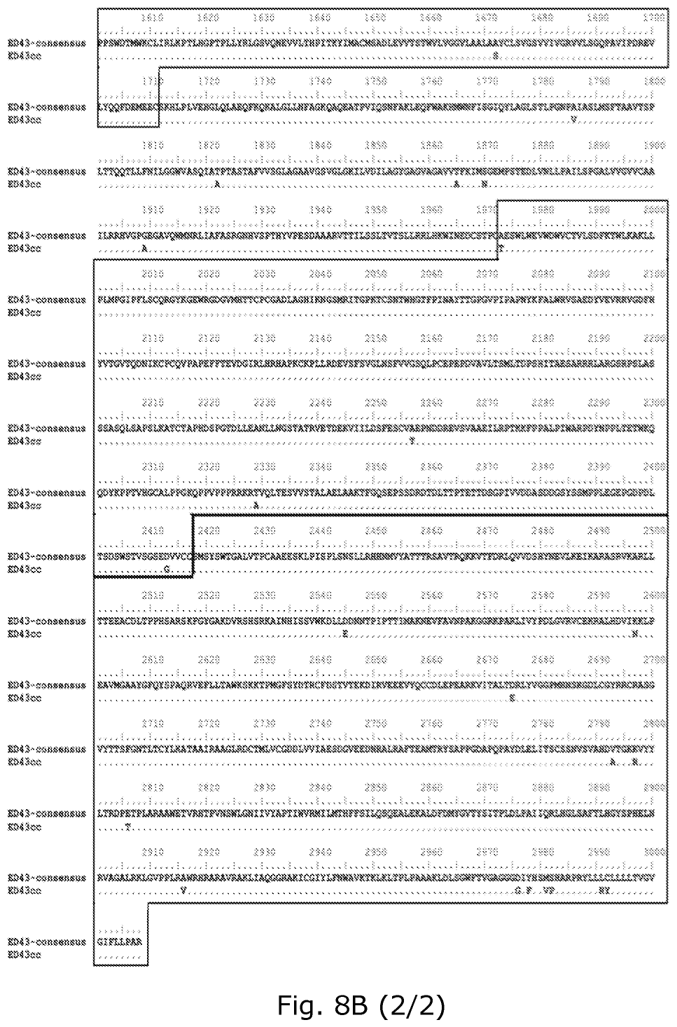

For adaptation of pED43 (Gottwein et al. 2010), we generated recombinants harboring 9 substitutions from ED43(C5A)-9m, and 6 NS5B-substitutions (A499V, Q514R, D559G, Y561F, L574R, C575Y), previously used for culture adaptation of genotypes 1a, 2a, 2b, 3a, and 6a (FIG. 1A) (Ramirez et al. 2018). In addition to these 15 changes, we used additional NS5B substitutions and produced two different recombinants: one with three NS5B changes (D128E, D258E, and M564V; ED43-18m) or with five NS5B changes (adding K380R and E389T creating ED43-20m) (FIG. 1A). These NS5B changes were selected based on the comparative analysis of polymerase sequences from ED43 and other strains for which infectious cultures were developed. This analysis led to the identification of residues that were different, which we hypothesized might be important for viral viability in cell culture (FIG. 8A). Following transfection, ED43-18m was non-viable, but ED43-20m spread at day 41 (2.6 log₁₀ FFU/mL). The infectivity titers increased during consecutive passages, reaching ˜5.0 log₁₀ FFU/mL at passage 10 (FIG. 1B; Table 1).

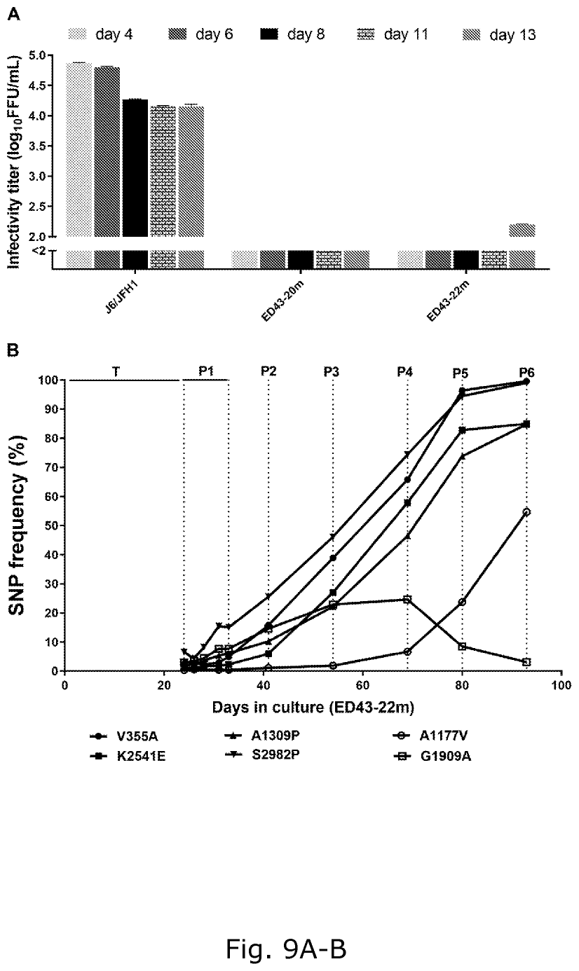

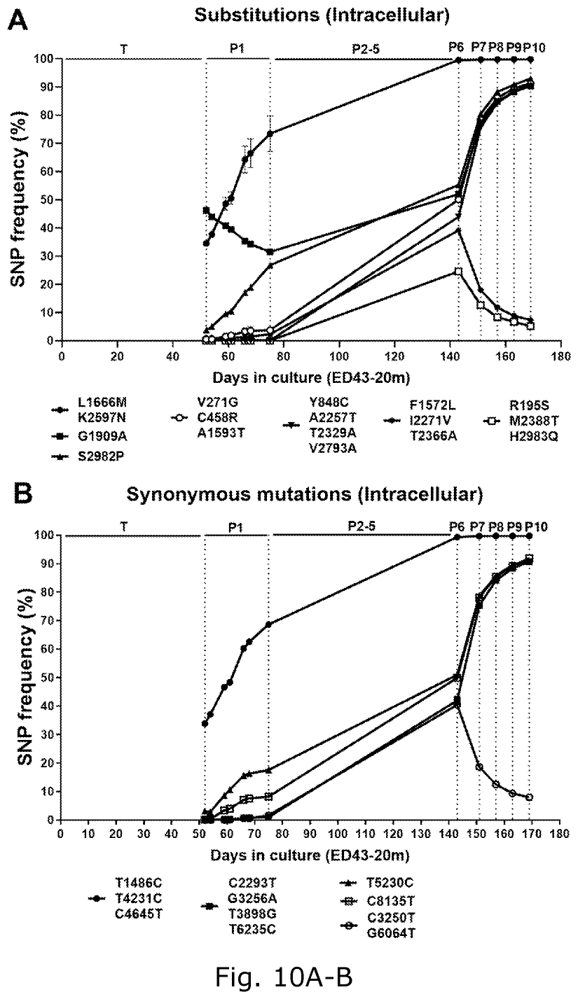

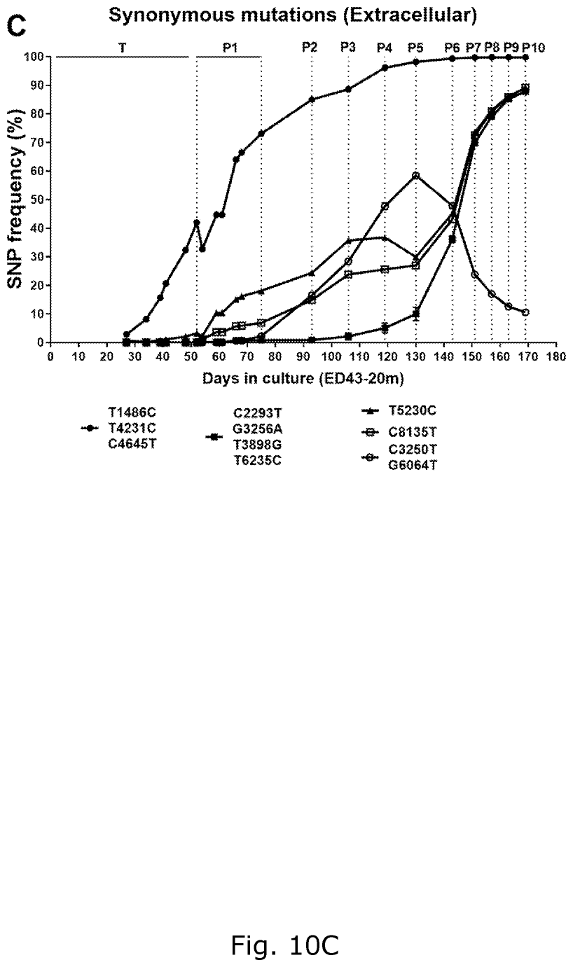

To identify substitutions evolving during serial passage of ED43-20m in Huh7.5 cells, we analyzed extracellular viral RNAs using NGS. The substitutions that emerged with similar patterns were grouped as shown in FIG. 1B. L1466M (NS3) and K2597N (NS5B) were found in >80% of the viral population at 2^{nd} passage (FIG. 1B). However, the fitness of a recombinant with these substitutions (ED43-22m) remained low and the virus acquired additional changes (FIG. 9). Thus, we investigated other substitution patterns (FIG. 1B). As shown in FIG. 1B, two different viral populations evolved that showed relatively equal prevalence in passage 6. However, one population became dominant in passage 10. The step-wise-acquisition of substitutions was confirmed by further analysis of 6^{th}- and 10^{th} passage viruses (FIG. 1C), by Sanger sequencing analysis of TA clones of amplicons. Clones were analyzed by linkage and ancestral reconstruction based on phylogeny. L1466M and K2597N were observed in all clones, following by the emergence of two different viral populations. Interestingly, we found that the mutation patterns were similar between cell-free and cell-associated viruses (FIG. 1B; FIG. 10).

Based on the evolution during ED43-20m adaptation, we constructed a recombinant harboring all dominant 10^{th} passage substitutions, except for A1973T (NS5A) (FIG. 1A, B). We also introduced F1572L, detected in the ED43(C5A)-7m recovered virus and emerging during the first 5 passages of ED43-20m (FIG. 1A,B; Table 3). The resulting ED43-31m spread fast and produced ˜4.3 log₁₀ FFU/mL (FIG. 1A, D). We did not detect other substitutions at >5% of the viral population in the 2^{nd} passage (Table 1). However, the titers of this virus did not match those observed at passage 10 of ED43-20m, and thus there was room for further optimization.

Among the original 20m substitutions, Q2931R and C2992Y had partly reverted (Table 1), and R2931Q was found to increase ED43-31m titers after transfection (FIG. 1D). In addition, titers of ED43-31m further increased after introducing A1973T (NS5A) (detected in the ED43-20m 10^{th} passage virus, but not initially included in ED43-31m) (FIG. 1D). Thus, we generated an ED43-31m recombinant containing A1973T and the reversion of Q2931R (ED43-31m/+A1973T/-Q2931R; or ED43-31m_opt), which showed fast spread kinetics and slightly higher ˜4.6 log₁₀ FFU/mL post-transfection titers (FIG. 1E).

Finally, we found that a recombinant carrying the G38A mutation in the 5′UTR (ED43-31m_opt with 5′UTR G38A, designated ED43cc), which was detected in the 10^{th} passage of ED43-20m, produced the highest titers mimicking the ED43-20m virus 10^{th} passage, ˜4.7 and 5.1 log₁₀ FFU/mL in transfection and 2^{nd} passage, respectively. We did not detect additional substitutions emerging at >5% of the viral population in the 2^{nd} passage virus (Table 1).

Overall, we revealed unique evolutionary details on HCV culture adaptation and developed full-length 4a recombinants that propagated robustly in Huh7.5 cells. The ED43cc harbored 32 changes compared to the consensus ED43 clone, including 5, 4, and 13 coding changes in NS3/4A, NS5A, and NS5B, respectively (FIG. 8B).

HCV evolutionary Genetic Networks Resulting in DAA-Resistance for Genotype 4a

The recommended DAA-based regimens for patients with chronic genotype 4 infection include NS3-protease (grazoprevir, paritaprevir, and glecaprevir), NS5A (elbasvir, ledipasvir, ombitasvir, velpatasvir, and pibrentasvir) and NS5B-polymerase (sofosbuvir) inhibitors. The evolutionary features underlying the emergence of viral resistance are not well characterized. The HCV genotype 4a infectious culture system can serve as a valuable model to explore determinants of virus-escape from DAAs. We thus performed NGS and linkage analysis permitting detailed investigation of emerging RASs during culture escape experiments, and examined the evolution of putative fitness compensating substitutions throughout the HCV genome using reverse genetics (Pham et al. 2018; Jensen et al. 2019; Pham et al. 2019).

### Evolutionary Pathways Underlying Emergence of RASs During Treatments With Protease-Inhibitors (PIs)

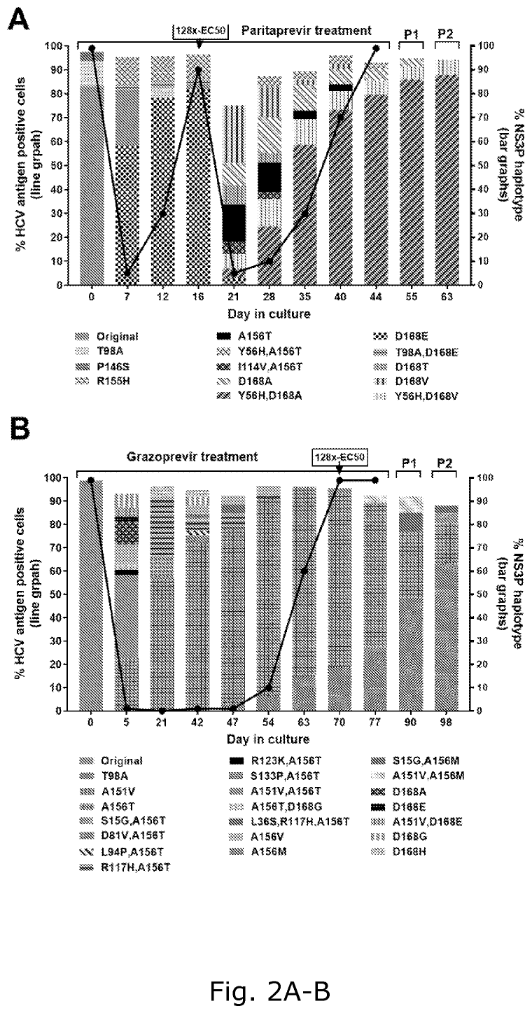

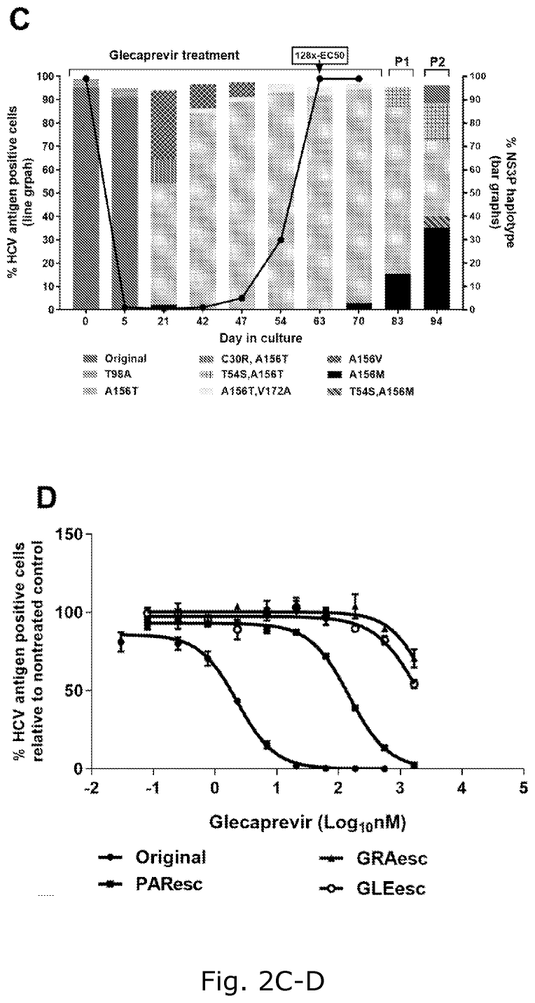

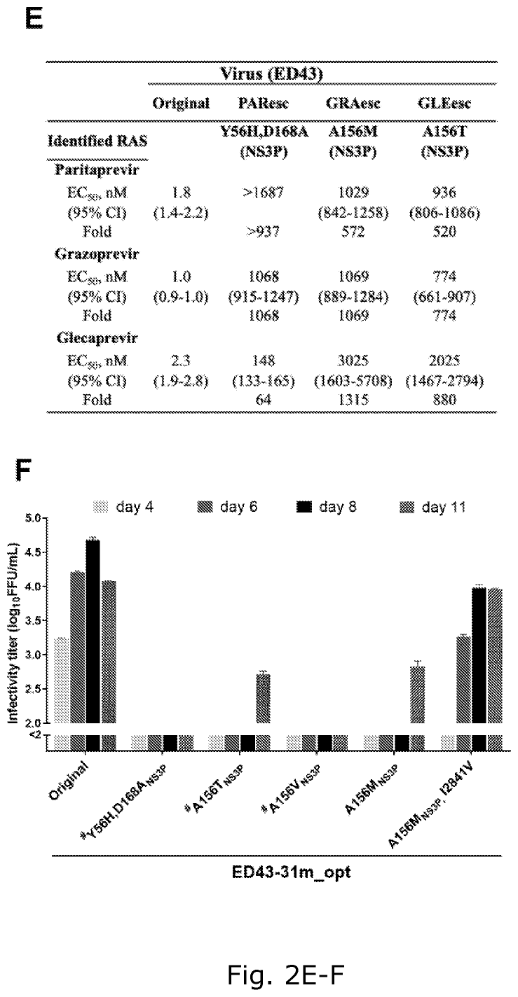

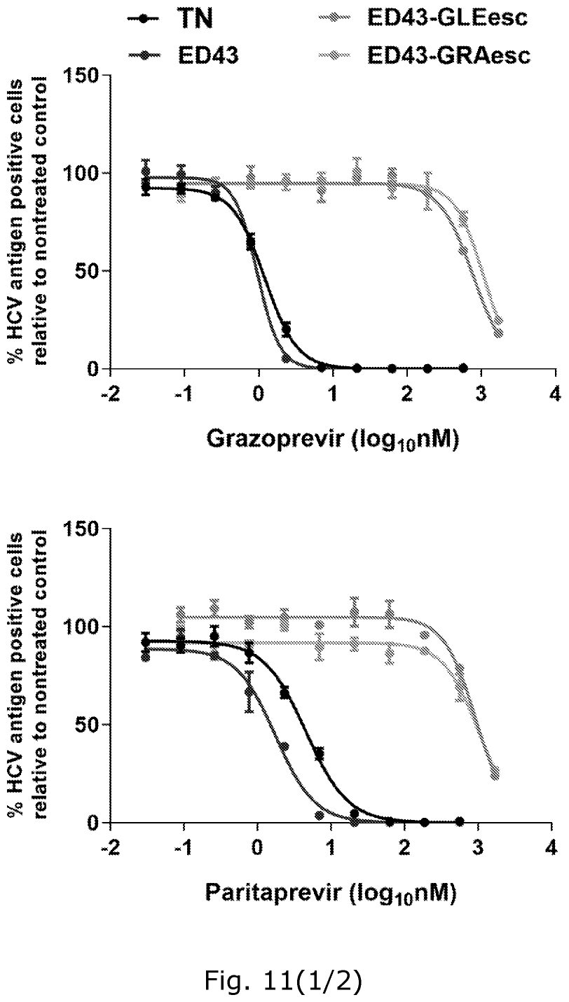

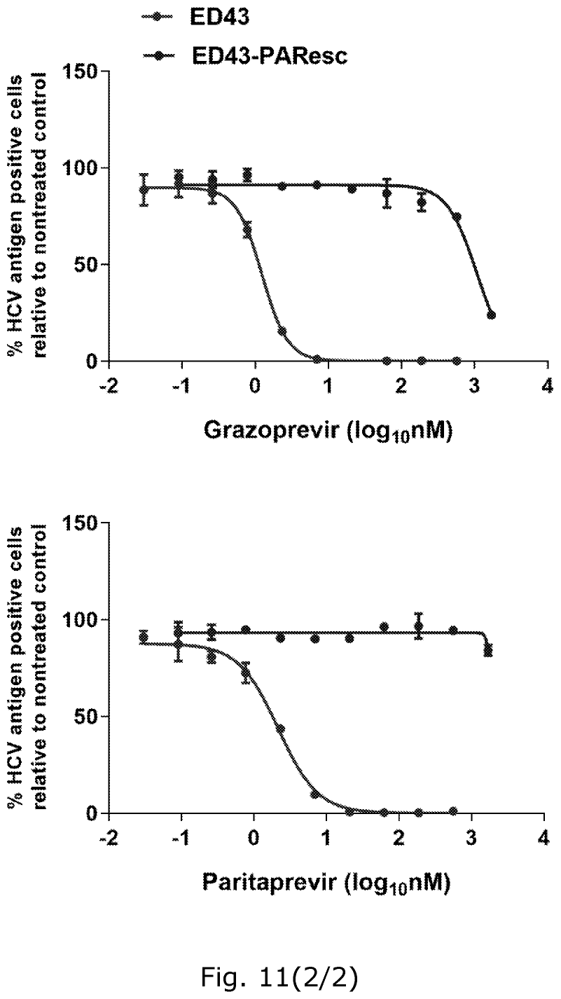

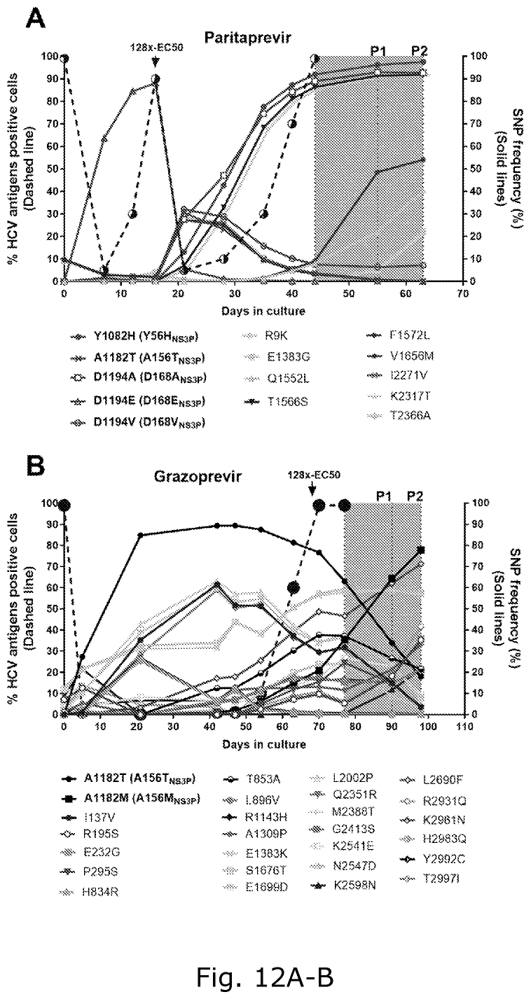

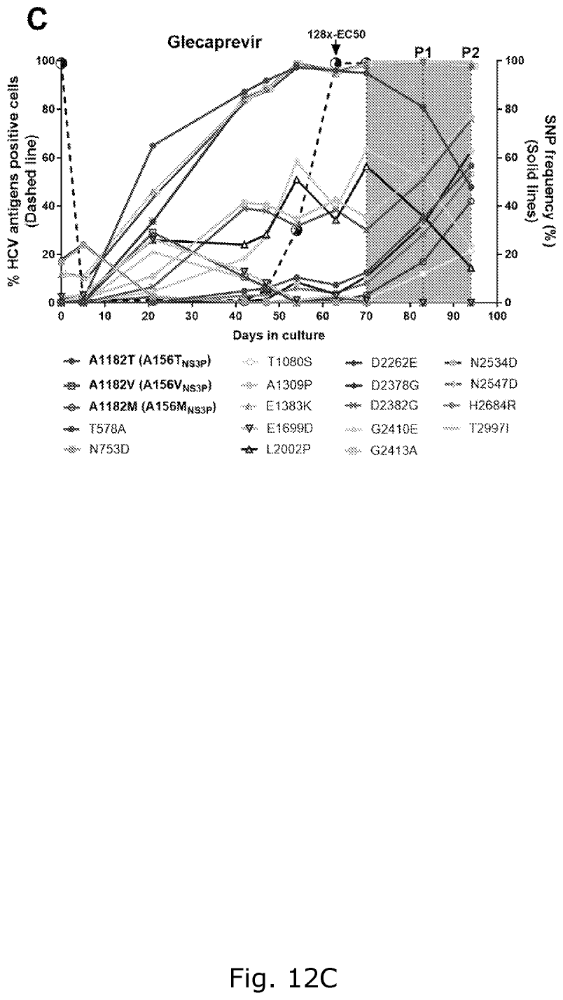

To induce viral escape from PIs, we performed long-term treatments of ED43-virus with paritaprevir, grazoprevir or glecaprevir at concentrations equivalent to 8×EC₅₀ (Jensen et al. 2019, Pham et al. 2019) (FIG. 2A-C). For paritaprevir, NS3P-D168E emerged at day 7 after treatment initiation and became a major RAS when the virus escaped at day 16. Under a higher inhibitor concentration (128×EC₅₀), D168E decreased in prevalence and different RASs singly or in combination emerged. Y56H+D168A appeared at low frequency at day 21 but became dominant following viral escape. The escape-virus (PAResc) showed cross-resistance to all tested PIs compared to the original virus with ≥64-fold increase in EC₅₀ (FIG. 2E; FIG. 11). However, an ED43 recombinant harboring Y56H+D168A was highly attenuated (FIG. 2F) and engineered RASs reverted after a 2^{nd} drug-free passage (Table 2). Therefore, compensatory substitutions might be required for stability of these RASs, which were maintained during 2^{nd} passage of the escape virus (FIG. 2A). NGS analysis of the complete viral ORF during treatment showed complex patterns of substitutions (FIG. 12A). T1566S[NS3-helicase (NS3H)] and K2317T(NS5A) emerged together with Y56H/D168A, suggesting linkage (FIG. 12A). In addition, Q1552L(NS3H) and V1656M(NS3H) evolved in drug-free passages suggesting a role in viral fitness (FIG. 12A).

Similarly, we investigated patterns of RASs under grazoprevir and glecaprevir treatments. After treatment initiation, different RASs developed, including A156T/V (FIG. 2B,C). However, viral spread did not occur until after day 53, even though A156T emerged to >50% by day 21. Viral escape was associated with T156 being replaced by M156. The grazoprevir (GRAesc) and glecaprevir (GLEesc) escape viruses were highly resistant to PIs with >500-fold increases in EC₅₀ (FIG. 2D,E; FIG. 11). However, ED43 recombinants harboring engineered A156T/V/M were highly attenuated (FIG. 2F), and the A156T/V reverted after transfection (Jensen et al. 2019). A156M was maintained, but the virus acquired additional coding ORF changes, including I2841V(NS5B; >50%), G2413D(NS5A; >5%) and D2689G(NS5B; >5%), in 2^{nd} passage (Table 2). Further, during inhibitor treatments, we found complex dynamic networks of substitutions outside NS3P (FIG. 12B,C). We detected 12841V, seen in the recombinant A156M virus, at 5% frequency in the GRAesc virus 2^{nd} passage. Interestingly, the A156M recombinant virus harboring I2841V was efficient and genetically stable (FIG. 2F, Table 2).

### Evolution Induced by NS5A-Inhibitors

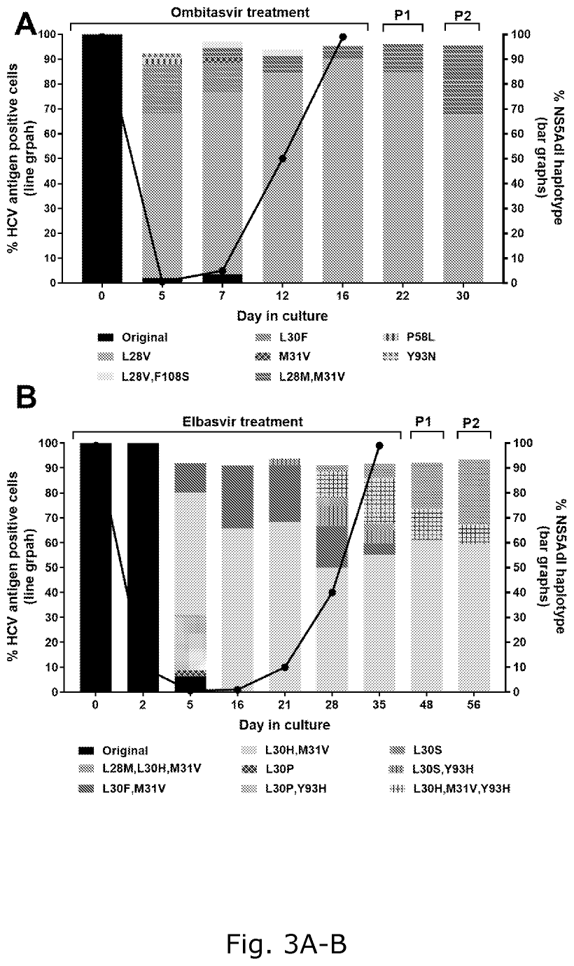

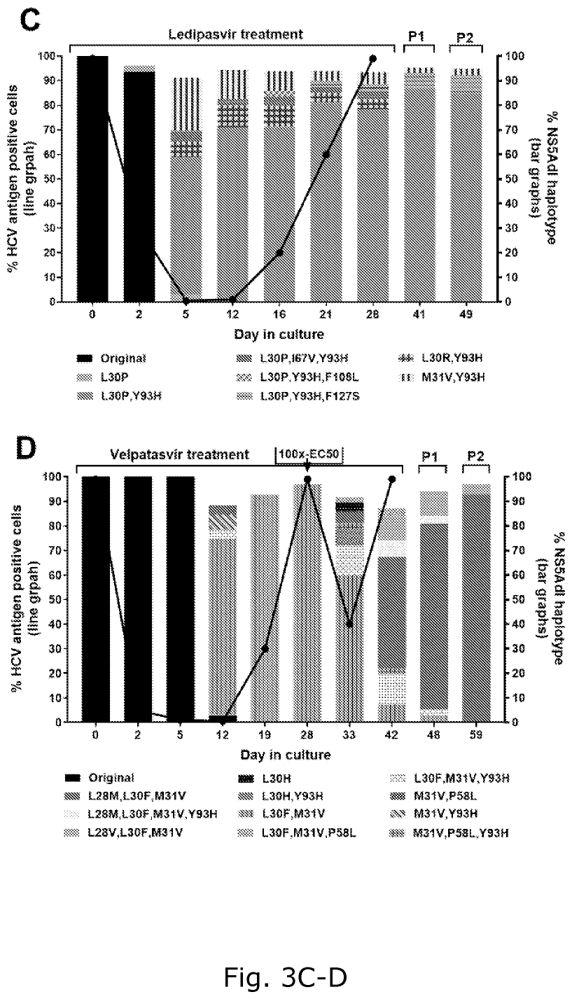

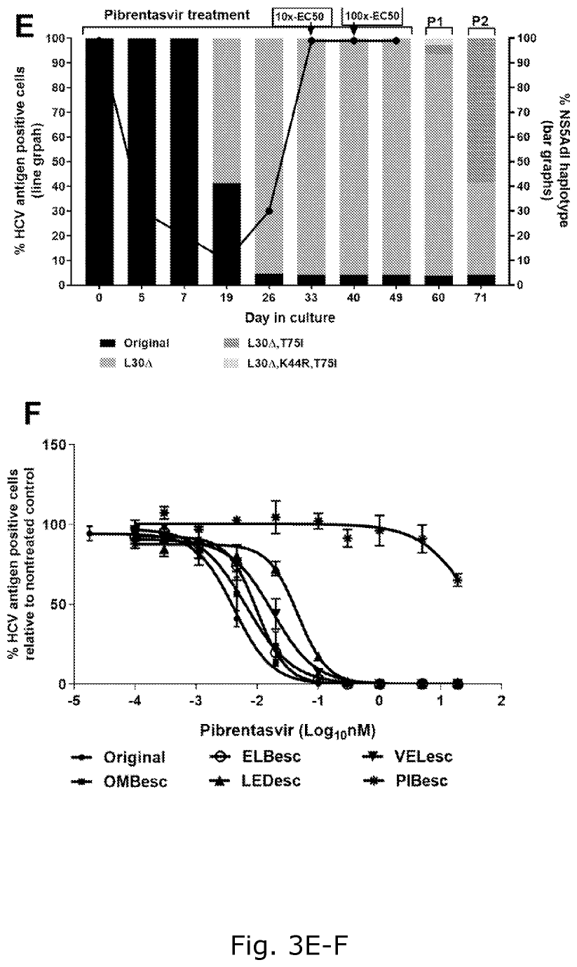

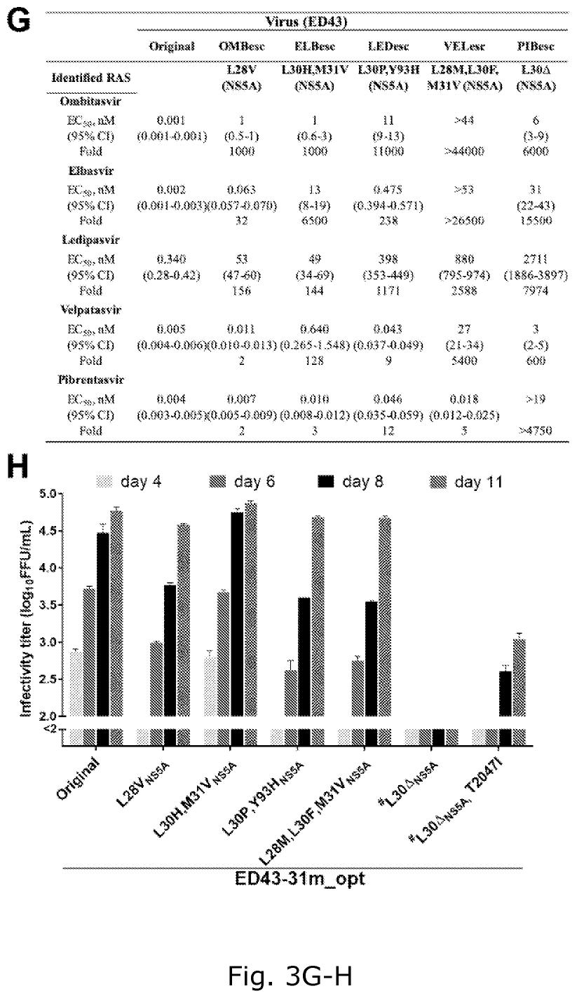

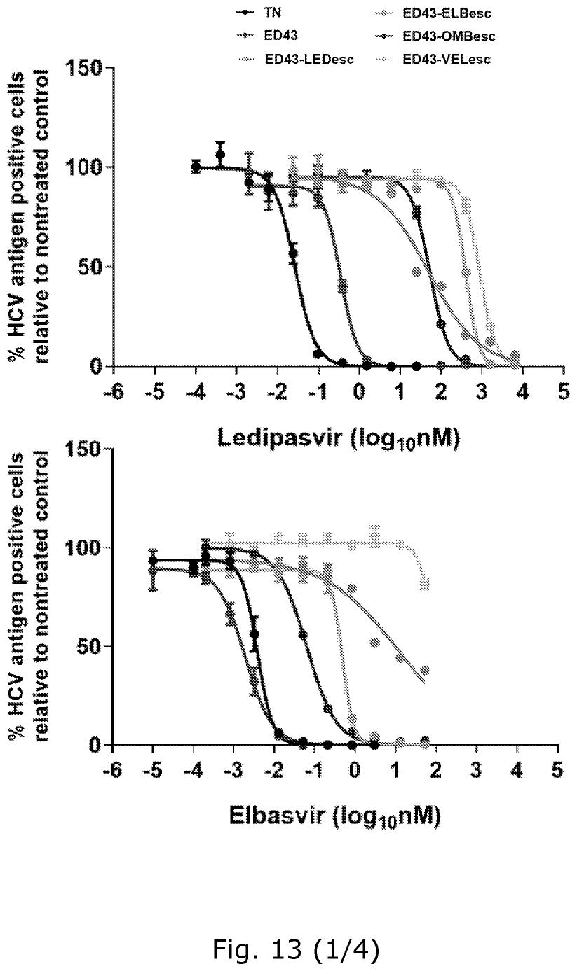

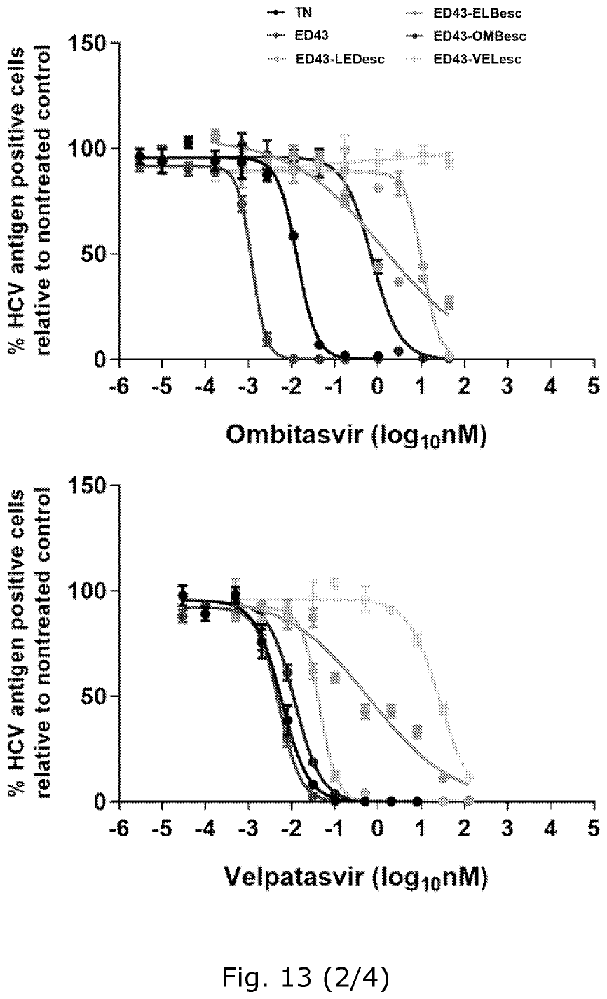

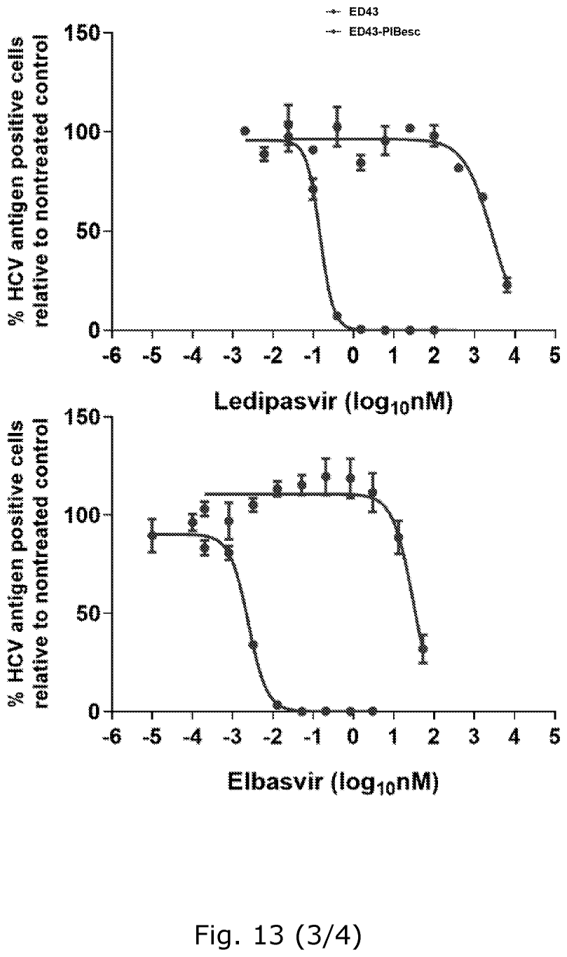

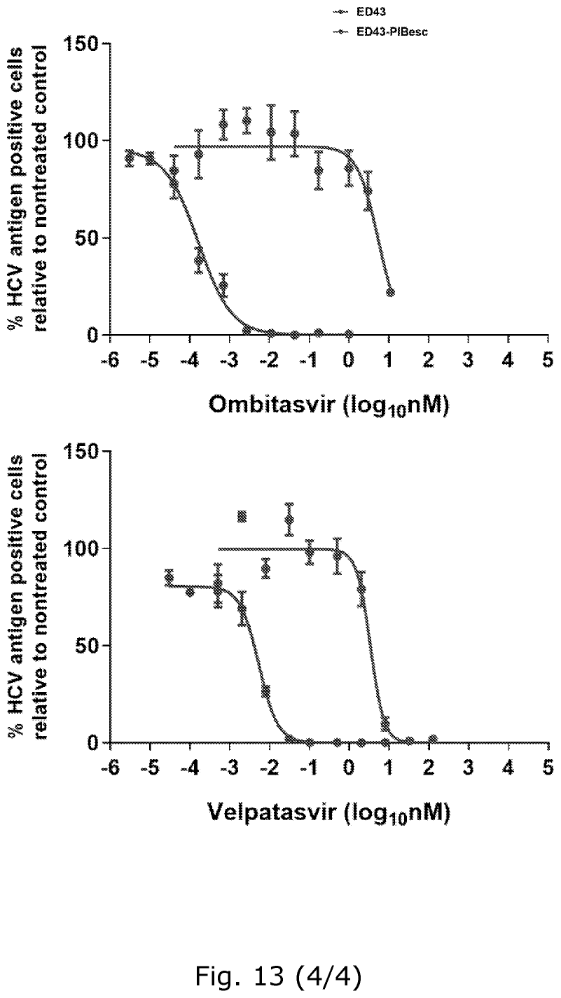

Under ombitasvir, elbasvir, and ledipasvir treatments (concentrations equivalent to 100×EC₅₀), the main RASs responsible for ED43 escape emerged at day 5 (FIG. 3A-C) (Pham et al. 2018). Following viral escape from ombitasvir (OMBesc), elbasvir (ELBesc), and ledipasvir (LEDesc), RASs conferring high-level resistance (FIG. 3G; FIG. 13) became dominant (FIG. 3A-C). The main NS5A RASs L28V (ombitasvir), L30H+M31V (elbasvir), and L30P+Y93H (ledipasvir) did not induce high fitness-cost when introduced in the ED43 recombinant and were maintained after 2^{nd} passage (FIG. 3H; Table 2).

For velpatasvir, we did not observe viral escape with 100×EC₅₀, and performed an experiment at 10×EC₅₀. The major RASs L30F+M31V was not acquired as rapidly as for ombitasvir, elbasvir, and ledipasvir, suggesting higher barrier to resistance (FIG. 3D).

These RASs were dominant in escape viruses until day 28. Here, the concentration was increased to 100×EC₅₀, resulting in diversification of the viral population, associated with increased viral suppression (FIG. 3D). L28M+L30F+M31V emerged as a major population, which led to viral escape (VELesc) and high resistance levels to NS5A inhibitors (except pibrentasvir) (FIG. 3F,G; FIG. 13). The ED43 L28M+L30F+M31V recombinant was highly fit and maintained the RASs after 2^{nd} passage (FIG. 3H, Table 2).

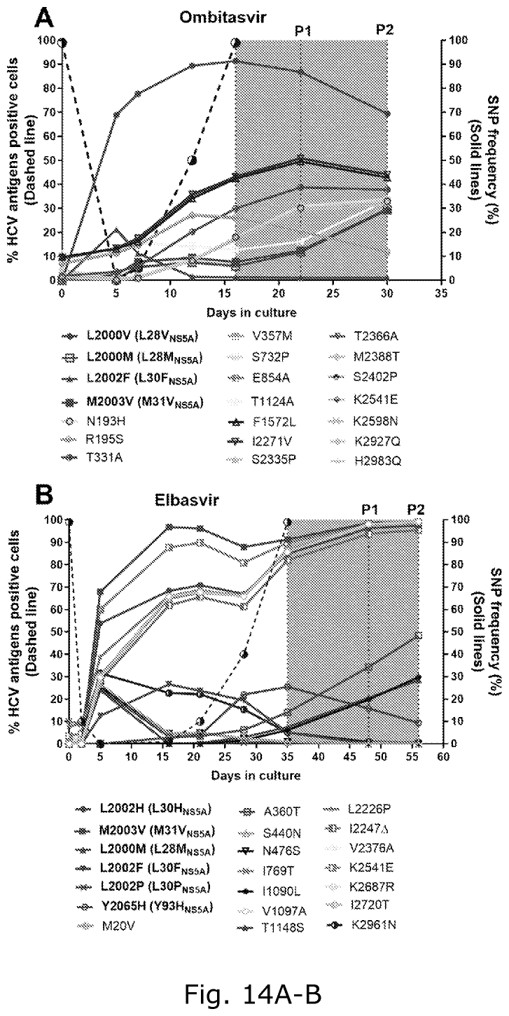

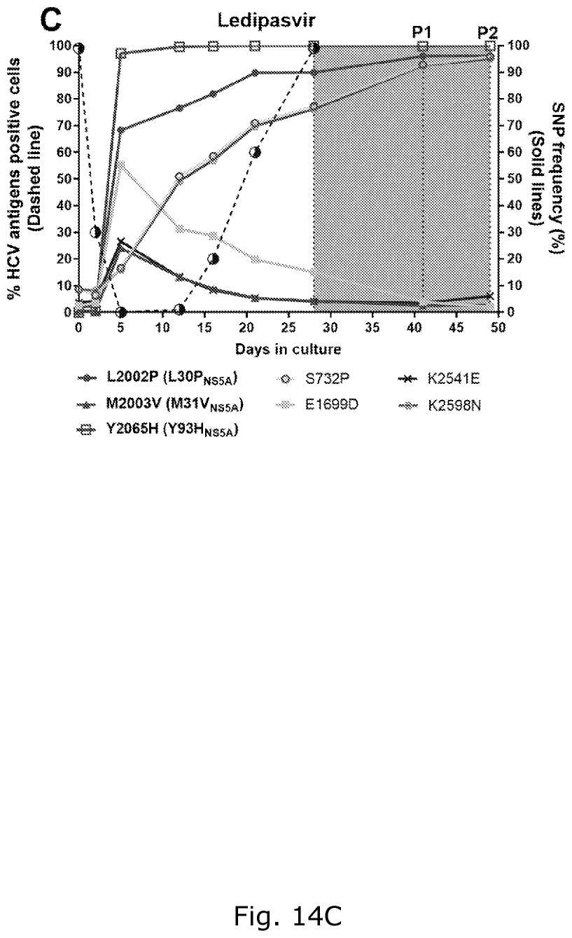

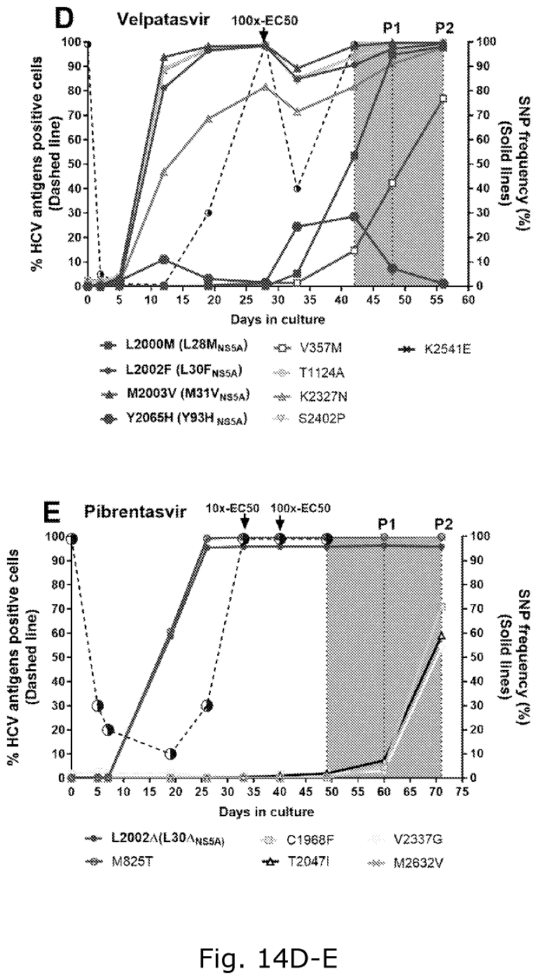

Genome-wide NGS showed that substitutions outside NS5A-domain I emerged in viruses escaping ombitasvir, elbasvir, ledipasvir and velpatasvir (FIG. 14A,B,C,D).

Pibrentasvir 10×EC₅₀-treatment resulted in viral eradication. However, 5×EC₅₀-treatment led to escape by day 33 (FIG. 3E). Interestingly, we primarily observed NS5A RAS L30Δ(deletion) (FIG. 3E), and no viral suppression was observed at increased concentrations (10×- and 100×EC₅₀), indicating that L30Δ conferred high resistance. Indeed, the escape virus (PIBesc) showed high levels of resistance to all NS5A-inhibitors (FIG. 3F,G; FIG. 13). In contrast to other NS5A-RASs, the ED43-L30Δ recombinant was highly attenuated, and acquired additional substitutions after 2^{nd} passage (FIG. 3H; Table 2). One of these substitutions, T2047I (T75I, NS5A-numbering), also found in the 2^{nd} drug-free passage of PIBesc, increased viability of the L30Δ recombinant (FIG. 3E,H; FIG. 14E).

### Evolution Induced by Sofosbuvir

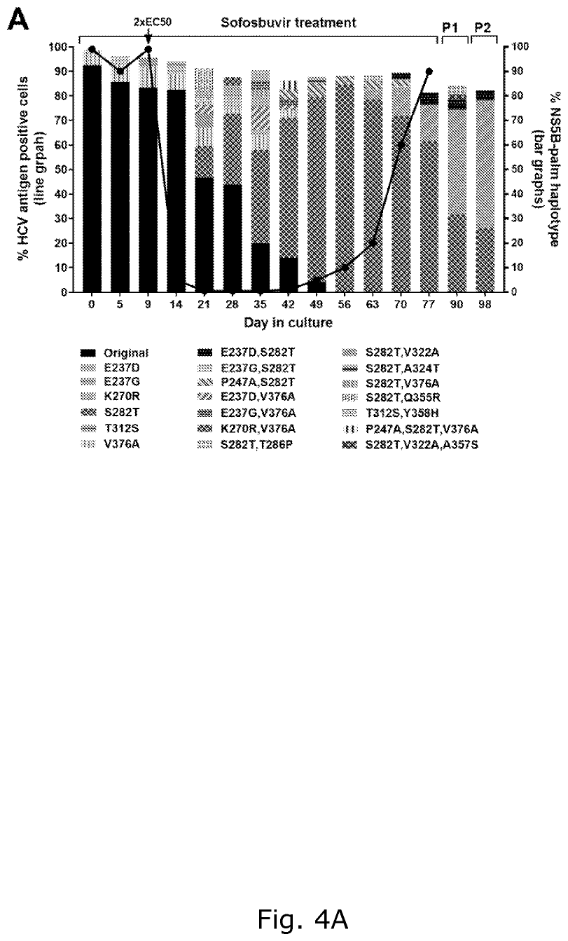

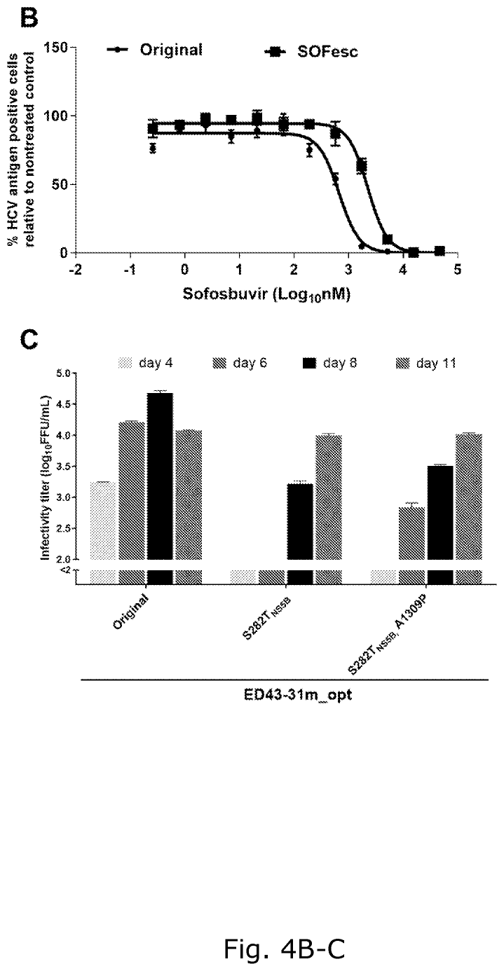

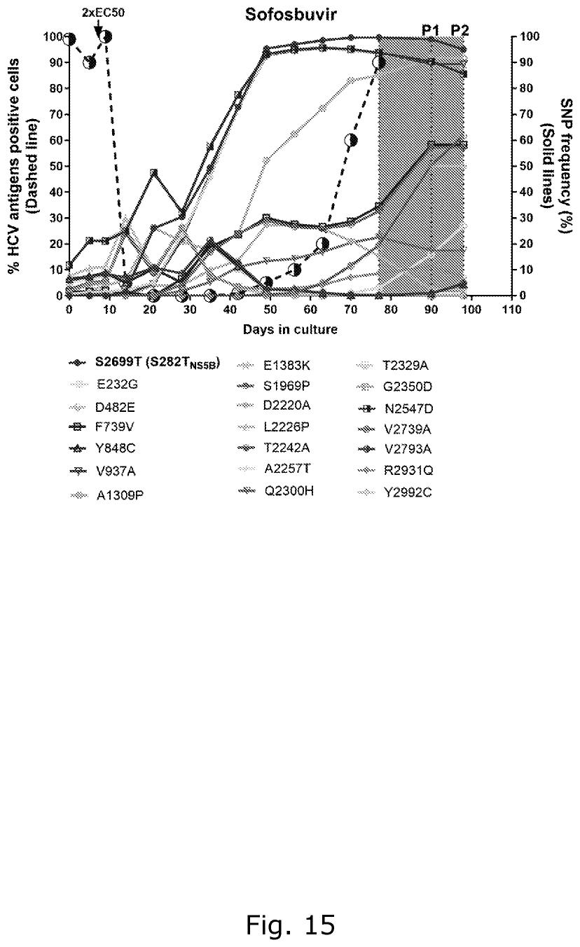

The ED43-virus treated with sofosbuvir was initially suppressed at 2×EC₅₀ but escaped at day 77 (FIG. 4A). NGS analysis of HCV-ORF during treatment highlighted a complex network of co-selection of substitutions underlying the gradual emergence of NS5B-RAS S282T (FIG. 15), co-existing with S282T+V322A (FIG. 4A). Interestingly, the S282T+V322A population increased during drug-free passages (FIG. 4A). The SOFesc virus had decreased sensitivity to sofosbuvir compared with original virus (EC₅₀ (nM [95% CI]): 2253 (1951-2622) versus 668 (545-806)) (FIG. 4B). Strikingly, the ED43 recombinants harboring engineered S282T with or without A1309P(NS3H), which developed following the emergence of S282T (FIG. 15), were relatively fit (FIG. 4C) and S282T was maintained after 2 consecutive drug-free passages (Table 2).

### Efficacy of Clinically Relevant DAA Combinations Against Genotype 4a

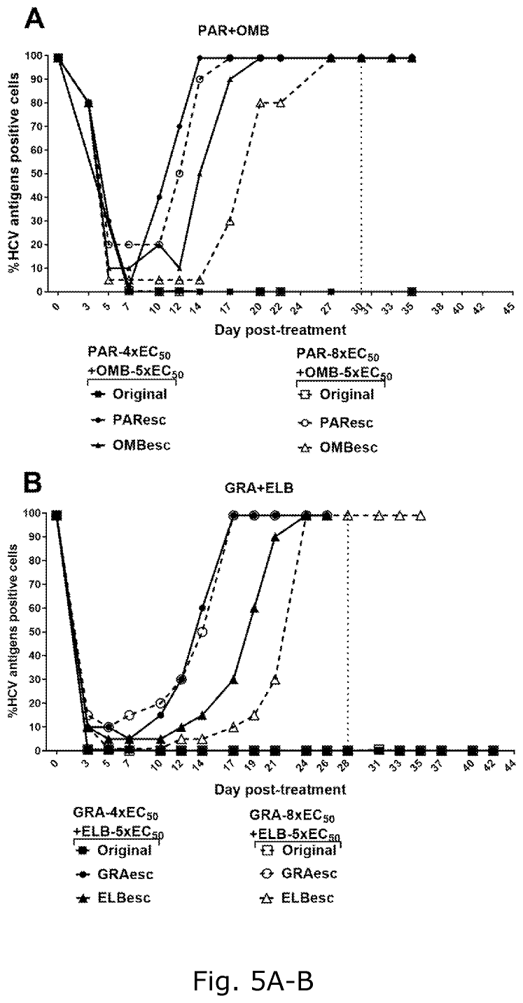

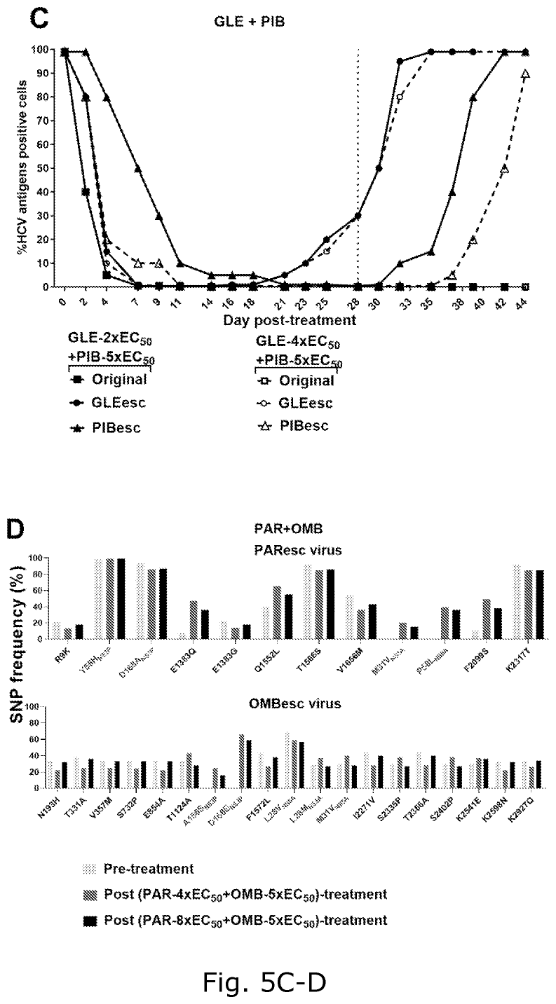

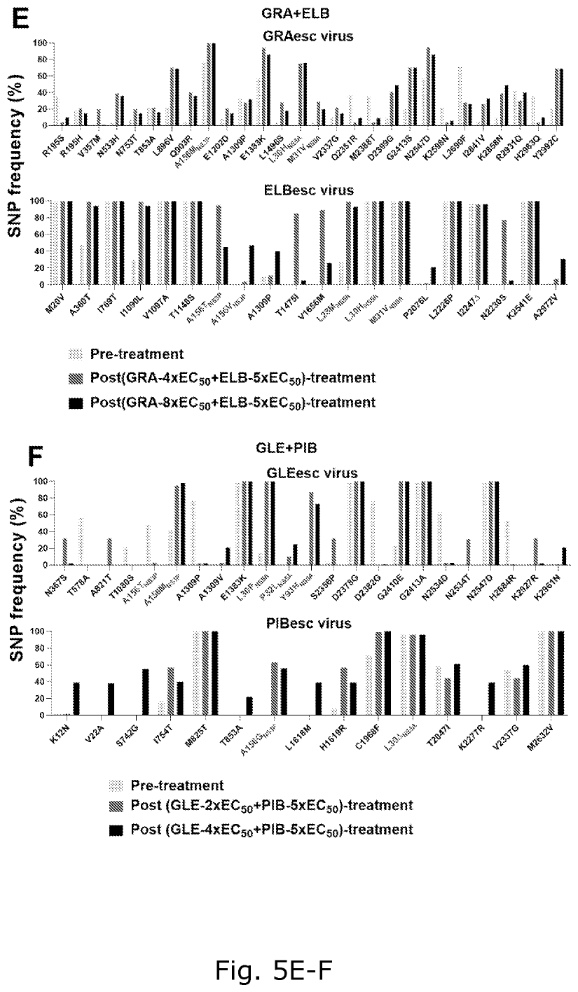

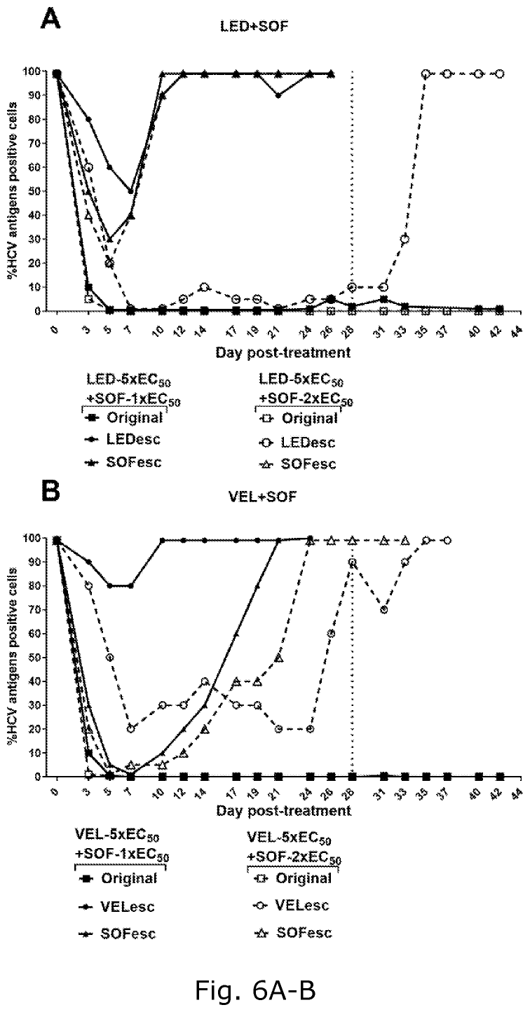

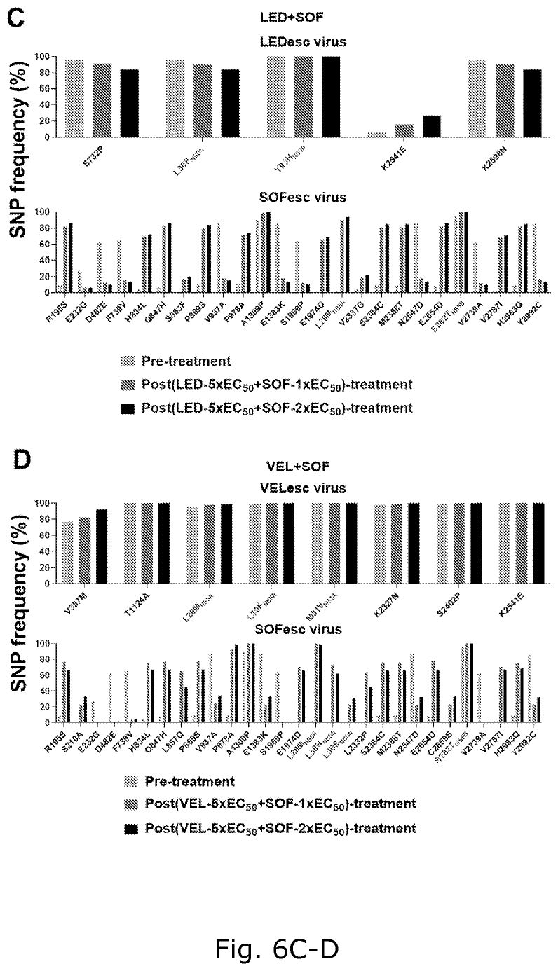

Recommended DAA combinations include paritaprevir/ombitasvir, grazoprevir/elbasvir, ledipasvir/sofosbuvir, velpatasvir/sofosbuvir, and glecaprevir/pibrentasvir. These regimens were efficient in suppressing the original ED43-virus (FIGS. 5 and 6). Except for treatment with LED (5×EC₅₀)/SOF (1×EC₅₀), where we consistently detected HCV-positive cells, the original virus was eradicated in all treatments. Next, we tested whether DAA combinations remained efficient against their corresponding single drug escape ED43-variants.

DAA regimens based on PIs (paritaprevir, grazoprevir, or glecaprevir) and NS5A inhibitors (ombitasvir, elbasvir or pibrentasvir) were inefficient against viruses that had escaped one of the included drugs, and thus harbored RASs at baseline (FIG. 5A,B,C) (Pham et al. 2018; Ramirez et al. 2016; Jensen et al. 2019). Only glecaprevir/pibrentasvir was able to control the GLEesc and PIBesc viral infections during treatment (FIG. 5C). In contrast, paritaprevir/ombitasvir and grazoprevir/elbasvir combinations were inefficient to suppress infections with PI or NS5A resistant viruses (FIG. 5A, B), which escaped after 2 weeks of treatment.

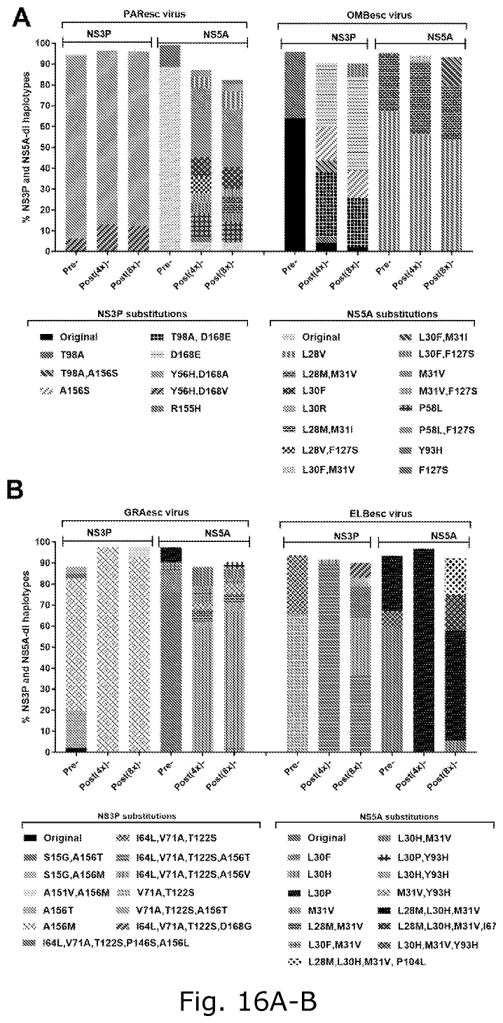

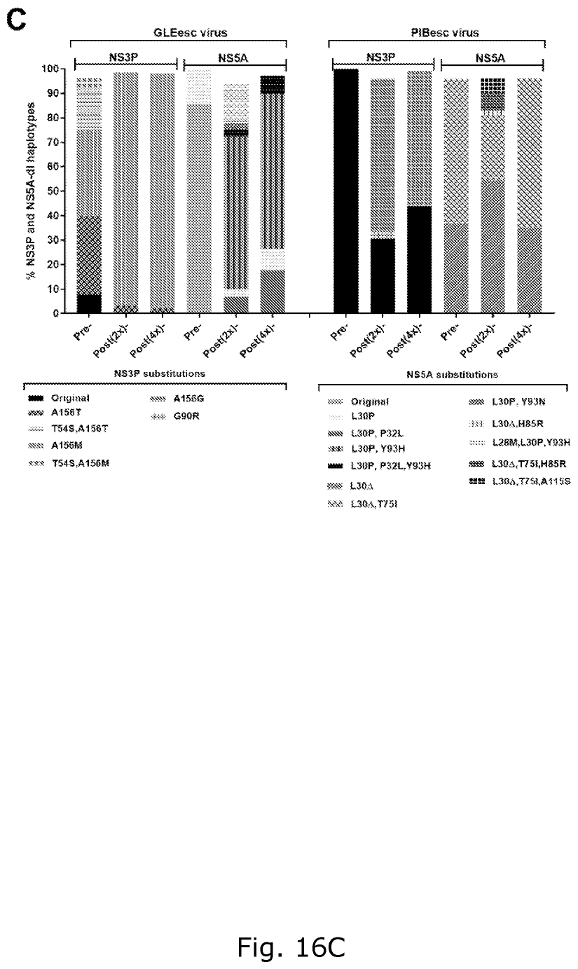

NGS and linkage analysis of viruses escaping from these combination treatments showed that in addition to the RAS at baseline, they all acquired RASs in the new target (FIG. 5D,E,F; FIG. 16). Moreover, additional substitutions outside the drug targets emerged after combination treatments (FIG. 5D,E,F). Particularly, 12841V emerged in the GRAesc virus containing NS3P-A156M.

Similarly, we tested the DAA combinations of NS5A (ledipasvir or velpatasvir) and NS5B (sofosbuvir) inhibitors against the respective LEDesc, VELesc and SOFesc viruses and found that those viral infections were not eradicated (FIG. 6A,B). The treatment with velpatasvir/sofosbuvir resulted in high suppression and delayed spread of the SOFesc virus as compared with ledipasvir/sofosbuvir.

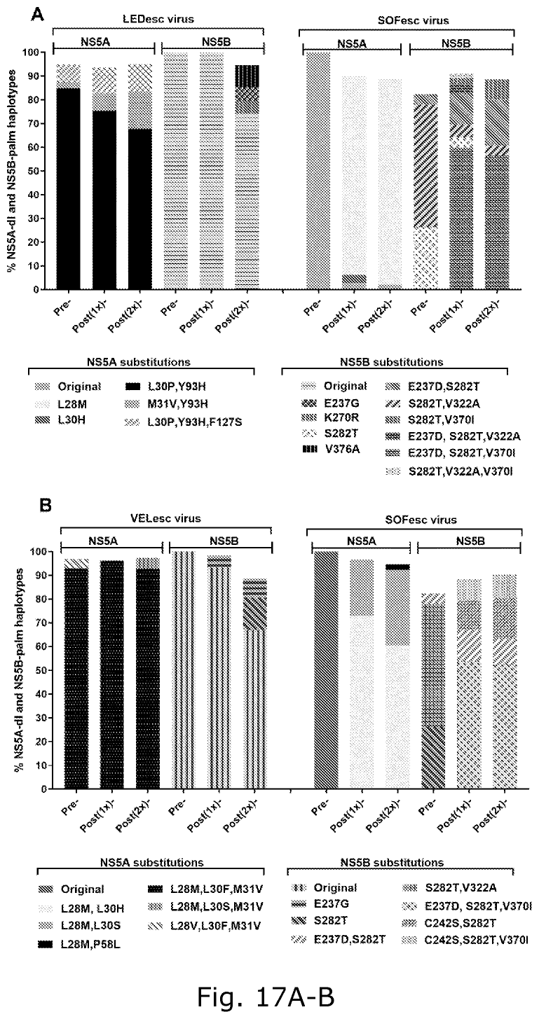

After combination treatments, the original LEDesc and VELesc viruses maintained the NS5A RASs (FIG. 6C,D; FIG. 17). The original SOFesc virus acquired NS5A L28M, L30H, and L30S, while maintaining NS5B-S282T, and acquiring additional substitutions throughout the ORF (FIG. 6C,D).

### Glecaprevir/Pibrentasvir as a Re-Treatment Option for HCV Genotype 4a With Baseline Resistance in Culture

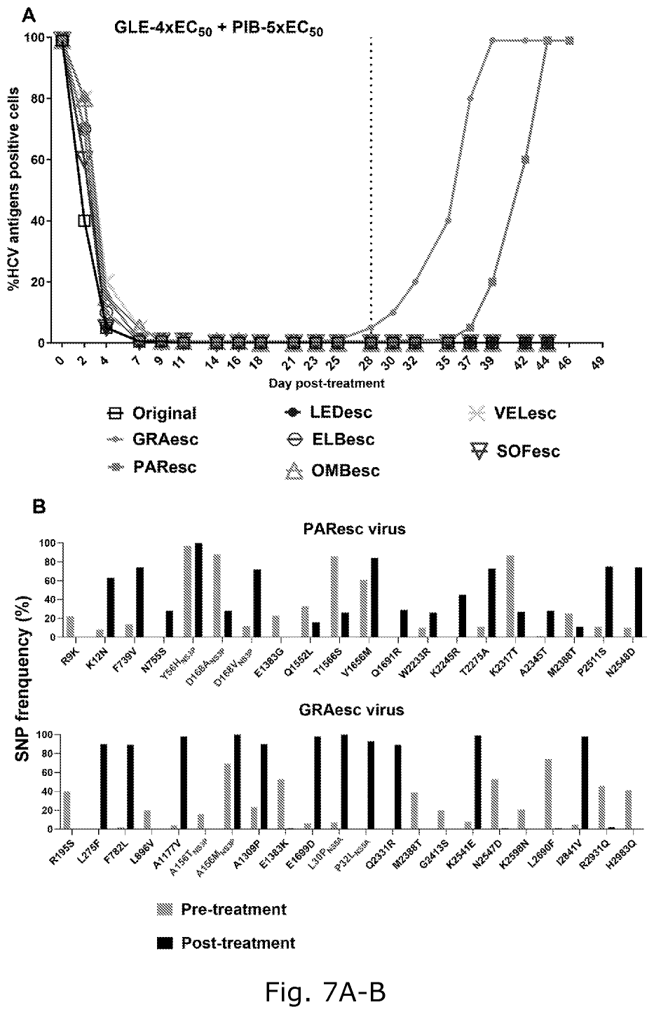

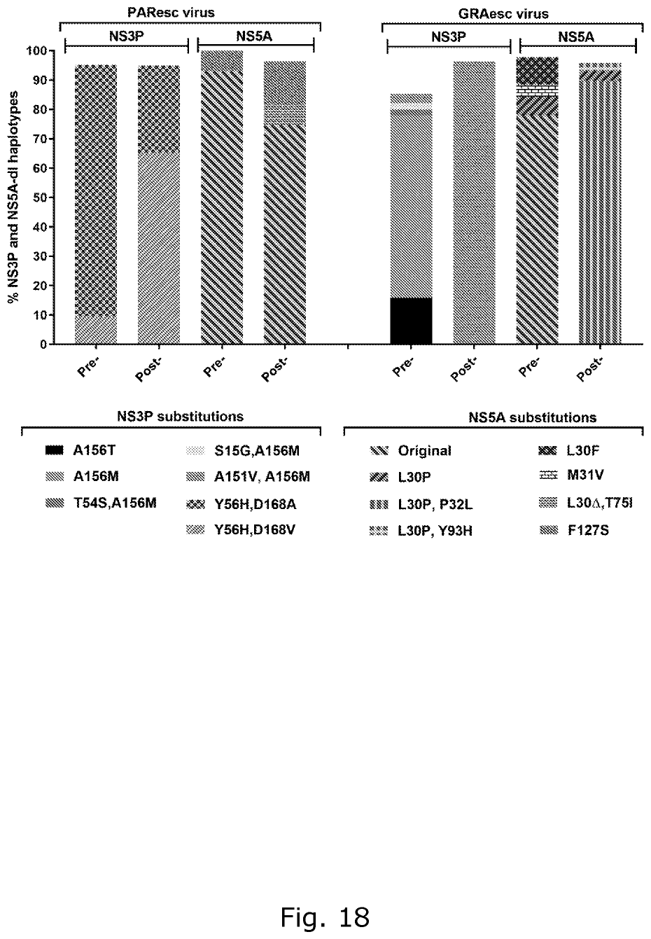

Our data demonstrated that DAA combinations could not eradicate infections with viruses resistant to one of the included inhibitors. Clinically, glecaprevir/pibrentasvir has been investigated as a re-treatment option for HCV-infected patients who failed DAA-containing regimens. Thus, we investigated its effectiveness against PAResc, GRAesc, OMBesc, LEDesc, ELBesc, VELesc, and SOFesc viruses. Infected cultures were treated with 4×EC₅₀ glecaprevir/5×EC₅₀ pibrentasvir. The infection was suppressed by day 7 of treatment and all except for PAResc and GRAesc viruses were eradicated after 28 days of treatment (FIG. 7A). NGS analysis of glecaprevir/pibrentasvir escape viruses showed that the GRAesc virus mainly harbored NS3P-A156M (combined with NS3P-A151V) and NS5A−L30P+P32L (FIG. 7B). The PAResc virus maintained NS3P−Y56H+D168A/V and acquired NS5A−L30Δ+T75I (as a minor population) (FIG. 7B; FIG. 18). Among substitutions emerging outside NS3P and NS5A domain I, we also found 12841V (NS5B) in the GRAesc virus (FIG. 7B). Thus, baseline PI resistance compromised the effectiveness of glecaprevir/pibrentasvir.

### Discussion

We unraveled adaptation leading to the development of a high-titer full-length infectious culture system for HCV genotype 4, a major cause of chronic liver diseases in the Middle East and North/Central Africa. Using cell-culture adapted ED43(4a) viruses and detailed NGS combined with haplotype re-construction analysis, we showed that complex networks of RASs and other substitutions outside the drug targets evolved under DAA treatments, which resulted in positive selection of RASs inducing high levels of resistance. We further demonstrated that glecaprevir/pibrentasvir remained efficient as a re-treatment option against viruses that had escaped NS5A inhibitors or sofosbuvir, in culture. This is highly relevant, since most genotype 4 patients have been treated with an NS5A inhibitor combined with sofosbuvir.

Recently, an ED43 infectious system was reported by Watanabe et al. (Watanabe et al. 2020). This system was developed by using substitutions previously identified in ED43 replicons, and the final ED43 virus could produce infectivity titers of ˜3.5 log₁₀ FFU/mL after 39 days of infection (Watanabe et al. 2020). Here, we report a different strategy, which is based on the use of substitutions conferring both viral replication and propagation combined with high infectivity titers. Previously, we had succeeded in adapting genotypes 3a and 6a to efficiently grow in culture by initially combining substitutions identified in their corresponding C5A recombinants with changes in NS5B, which consisted in modifications of the sequence to reflect the consensus as compared with other genotype 3a and 6a sequences, respectively (Pham et al. 2018; Ramirez et al. 2016). However, for the original ED43, the NS5B sequence in the clone already reflected the consensus when compared to other deposited genotypes 4a sequences. Instead, we focused on the 10 amino acids of ED43-NS5B that differ from conserved sequences of other HCV genotype strains for which efficient infectious culture systems had been developed (FIG. 8A) (Ramirez et al. 2018), and found that a subset of these changes facilitated adaptation of the full-length ED43 clone. Our ED43cc virus was highly efficient and produced titers of ˜5.1 log₁₀ FFU/mL after 6 days of infection (Table 1). It should be noted that the ED43cc virus contains numerous substitutions identified through an extensive adaptation process. Thus, we cannot rule out that these adaptive substitutions could impact selection of viral resistance to DAAs. Among substitutions identified in our study and in Watanabe et al. (Watanabe et al. 2020), substitutions at positions 271 (V271F/G) and 2413 (D2413G) were commonly observed, suggesting that these changes might be important for the culture viability of this particular virus strain. It is worth mentioning that selection of viral resistance to DAAs and development of whole-virus vaccine candidates requires highly efficient viruses.

Genotype 4a ED43 replicon systems have been developed. Nevertheless, the cell culture adaptive mutations identified in this study could provide an alternative source to generate even more efficient genotype 4 sub-genomic replicons, as recently demonstrated for strain DBN3a of genotype 3a (Ramirez et al. 2016). Such replicons with or without RAS, recapitulating only the intracellular replication of the virus, are useful tools to study the effect of antivirals on replication but cannot be used to understand genomic-wide mutation networks.

Infectious cell culture systems can be important tools for vaccine development. The efficient growth of the ED43cc virus, with high infectivity titers, might permit the production of enough virus to generate inactivated whole virus vaccine candidates for pre-clinical testing. Alternatively, further cell-culture adaptation can be achieved through serial passage of ED43cc, as described previously for another recombinant (Mathiesen et al. 2015). Thus, the highly adapted ED43cc virus could contribute to the production of HCV virions needed in whole virus particle vaccine studies. Nevertheless, we must acknowledge a putative influence of the cell culture adaptive substitutions needed to grow ED43 in culture in the overall viral sensitivity to neutralizing antibodies, which could influence vaccine induced immune responses. Particularly, C458R (E2) has been shown to induce viral escape from host-immune responses. Furthermore, adaptive substitutions might also influence on viral sensitivity to DAAs that subsequently confer viral escape, however as the study of HCV in culture is dependent on adaptive mutations this is a universal limitation of cell culture systems.

We showed that heterologous ED43 viral populations containing different RASs evolved under various DAA treatments, which resulted in positive selection of RASs conferring high levels of resistance (FIGS. 2A-C; 3A-E; 4A). Also, the emergence and number of RASs did not depend only on the initial potency of the drug (EC₅₀). At a concentration of 8×EC₅₀, A156T/V/M emerged during treatments with grazoprevir and glecaprevir, but not with paritaprevir, suggesting higher selection pressure of glecaprevir and grazoprevir during long-term treatment despite similar potency (FIGS. 2A versus 2B and C). For NS5A-inhibitors a similar effect was observed, since despite exhibiting similar potency, ombitasvir selected a single RAS, while ledipasvir and elbasvir selected double RAS during long-term treatment (FIG. 3A versus 3C). The observed RASs L28V and L30R+Y93H also developed in genotype 4 infected patients after treatment failures with ombitasvir and ledipasvir containing regimens, respectively.

Additionally, our data suggests that viral escape heavily relied on the fitness of the corresponding RAS-containing ED43 variant. Most NS3P RASs were detrimental for viral fitness and reverted to wildtype, as also shown previously for other genotypes in culture (Jensen et al. 2019). For A156M, although it was maintained when introduced singly in ED43, compensatory substitutions were required to improve fitness (FIG. 2F). Clinically, this RAS has been detected in HCV-infected patients failing grazoprevir/elbasvir, including in genotype 4 infections.

The only NS5A RAS with a high fitness cost was NS5A-L30Δ, which could partly be compensated by NS5A-T75I (FIG. 3H). In line with the loss of fitness of L30Δ, we previously showed that an NS5A-P32 deletion had low fitness, but it conferred high-levels of resistance to all clinically relevant NS5A-inhibitors in genotype 1 viruses (Gottwein et al. 2018). In genotype 1 infected patients failing glecaprevir/pibrentasvir, NS5A-P32 deletion is also observed.

The NS5B-S282T is usually associated with high fitness-cost, thus it is rarely detected at baseline in HCV infected patients. However, it was reported that this RAS could be selected in culture under sofosbuvir treatment (Pham et al. 2018; Ramirez et al. 2016). We showed that S282T was gradually selected under sofosbuvir treatment of genotype 4a and maintained without drug pressure (FIG. 4A). In fact, compared to other genotypes, S282T is more frequently found in genotype 4 infected patients after DAA failures. In previous cell culture studies, we demonstrated that a genotype 6a recombinant harboring S282T exhibited severely impaired fitness, in contrast to the relatively fit 4a recombinant (FIG. 4C), suggesting differential effect of S282T among genotypes (Pham et al. 2018). This relative fitness advantage observed for genotype 4 could enable the S282T virus to accumulate additional substitutions for facilitating its long-term persistence after treatment failures. If the same occurs in patients, it could consequently decrease the barrier of resistance of sofosbuvir-containing regimens.

DAA combination treatments for genotype 4 have not been investigated in detail in culture. Our data showed that recommended DAA regimens were highly efficient against the original genotype 4 virus (FIGS. 5A-C and 6A-B). Similarly, these regimens are highly efficient in the clinic. Nonetheless, viral resistance to DAA combinations remains an issue, which could hamper treatment. In Egypt, treatment failures occur in 3-5% of genotype 4 infected patients. As treatment failure due to antiviral resistance is universally linked to NS5A-inhibitor resistance, a valid option for a salvage DAA regimen should include the pan-genotypic NS5A inhibitor pibrentasvir, which exhibits higher potency against most NS5A-resistant variants (Pham et al. 2019; Gottwein et al. 2018). Indeed, we showed that glecaprevir/pibrentasvir remained efficient against the 4a viruses harboring NS5A RASs (FIG. 7A). This effectiveness was likely due to the high potency of pibrentasvir, as shown by resistance profile testing (FIG. 3F) (Gottwein et al. 2018). Moreover, the virus harboring NS5B-S282T was also eradicated by this combination. This finding has important implications for patients failing regimens containing an NS5A-inhibitor combined with sofosbuvir, which have been used for the treatment of a high number of infected individuals in Egypt. In addition, since in this study the biggest loss of fitness in the ED43 virus was associated with the introduction of substitutions at NS3-156 and a deletion at NS5A-30, glecaprevir/pibrentasvir exhibits a high barrier to resistance. In fact, it was reported that patients failing treatment with an NS5A inhibitor and sofosbuvir were re-treated with glecaprevir/pibrentasvir, resulting in >90% SVR. Importantly, most patients had baseline NS5A RASs before re-treatment.

In our study, the viruses with NS3P RASs conferring high-level glecaprevir resistance, could not be eradicated by glecaprevir/pibrentasvir (FIG. 7A). Possible re-treatment options for these viruses could include velpatasvir/sofosbuvir, triple combinations of velpatasvir/sofosbuvir/voxilaprevir, or glecaprevir/pibrentasvir with the addition of sofosbuvir and/or ribavirin, which have shown great efficacy in patients. Therefore, it would be relevant to test these combinations against PI escape viruses in future studies.

In summary, we developed a highly efficient full-length HCV genotype 4a infectious culture system. Besides its use to improve our understanding about DAA resistance, this system could serve as a useful tool for the development of an HCV vaccine, which is urgently needed for control of HCV worldwide (Mathiesen et al. 2015). Here, we performed an extensive analysis of all clinically relevant DAAs that are currently being used for the treatment of genotype 4 infections. NGS and linkage analysis revealed complex dynamics operating in the selection of different RASs during treatments. The relatively high fitness and stability of NS5B-S282T observed in ED43 recombinants could have implications for the persistence of this RAS in genotype 4 infections after treatment with sofosbuvir-containing regimens. However, we showed that glecaprevir/pibrentasvir might be a promising salvage DAA regimen for the re-treatment of genotype 4 after failure with sofosbuvir/NS5A inhibitor-containing regimens. The detailed understanding of the evolutionary mechanisms underlying emergence of RASs generated here can contribute to efforts directed at avoiding the emergence and transmission of DAA-resistant viruses and thus to prevent treatment failure in the future.

### Tables:

TABLE 1
Non-synonymous ORF mutations in ED43 recombinant viruses during culture adaptation

| | | | | | | | | SNP frequencies (%) | | | | |
|---|---|---|---|---|---|---|---|---|---|---|---|---|
| | ED43 | | | | | | H77 | ED43-20m | | ED43-31m | ED43-31m_opt | ED43cc^{a} |
| HCV | NT | NT | | AA | AA | | AA | 4.1^{b} (4^{th}, | 5.0^{b} | 4.3^{b} | 5.1^{b} | 5.1^{b} |
| gene | pos | change | | pos | change | | pos | 13) | (10^{th}, 6) | (2^{nd}, 8) | (2^{nd}, 6) | (2^{nd}, 6) |
| E1 | 923 | C | A | 195 | R | S | 195 | 7.1 | 6.9 | — | — | — |
| E1 | 1152 | T | G | 271 | V | G | 271 | 28.1 | 89.1 | 99.9 | 100 | 100 |
| E2 | 1553 | T | C | 405 | S | P | 405 | 7.2 | — | — | | |
| E2 | 1712 | T | C | 458 | C | R | 458 | 26.2 | 88.9 | 99.9 | 100 | 100 |
| E2 | 2534 | T | C | 732 | S | P | 732 | 8.6 | — | — | — | — |
| p7 | 2597 | A | G | 753 | N | D | 753 | 16.8 | — | — | — | — |
| p7 | 2646 | T | C | 769 | I | T | 769 | 10.2 | — | — | — | — |
| p7 | 2730 | T | C | 797 | F | S | 797 | 11.5 | — | — | — | — |
| NS2 | 2819 | A | G | 827 | T | A | 827 | 100 | 100 | 100 | 100 | 100 |
| NS2 | 2883 | A | G | 848 | Y | C | 848 | 6.5 | 88.2 | 99.6 | 100 | 100 |
| NS3 | 3710 | A | G | 1124 | T | A | 1124 | — | 11.3 | — | — | — |
| NS3 | 4211 | A | G | 1291 | I | V | 1291 | 100 | 100 | 100 | 100 | 100 |
| NS3 | 4487 | G | A | 1383 | E | K | 1383 | 11.7 | — | — | — | — |
| NS3 | 4733 | A | G | 1465 | S | G | 1465 | 100 | 100 | 100 | 100 | 100 |
| NS3 | 4736 | T | A | 1466 | L | M | 1466 | 99.8 | 100 | 100 | 100 | 100 |
| NS3 | 5054 | T | C | 1572 | F | L | 1572 | 45.8 | 9.8 | 100 | 100 | 100 |
| NS4A | 5354 | G | T | 1672 | A | S | 1672 | 100 | 100 | 100 | 100 | 100 |
| NS4B | 5697 | C | T | 1786 | A | V | 1786 | 100 | 100 | 100 | 100 | 100 |
| NS4B | 5804 | A | G | 1822 | T | A | 1822 | 100 | 100 | 100 | 100 | 100 |
| NS4B | 5933 | A | G | 1865 | T | A | 1865 | 100 | 100 | 100 | 100 | 100 |
| NS4B | 5949 | G | A | 1870 | S | N | 1870 | 100 | 100 | 100 | 100 | 100 |
| NS4B | 6066 | G | C | 1909 | G | A | 1909 | 38.0 | 87.8 | 99.9 | 100 | 100 |
| NS5A | 6257 | G | A | 1973 | A | T | 1973 | 25.8 | 88.4 | — | 100 | 100 |
| NS5A | 7017 | T | C | 2226 | L | P | 2226 | 7.7 | — | — | — | — |
| NS5A | 7109 | G | A | 2257 | A | T | 2261 | 5.3 | 88.4 | 99.9 | 100 | 100 |
| NS5A | 7126 | C | A | 2262 | D | E | 2265 | 17.8 | — | — | — | — |
| NS5A | 7151 | A | G | 2271 | I | V | 2274 | 46.7 | 9.6 | — | — | — |
| NS5A | 7325 | A | G | 2329 | T | A | 2332 | 5.4 | 88.4 | 99.5 | 100 | 100 |
| NS5A | 7436 | A | G | 2366 | T | A | 2369 | 45.9 | 9.0 | — | — | — |
| NS5A | 7503 | T | C | 2388 | M | T | 2388 | 11.2 | 7.3 | — | — | — |
| NS5A | 7578 | A | G | 2413 | D | G | 2416 | 100 | 100 | 100 | 100 | 100 |
| NS5B | 7975 | C | A | 2545 | D | E | 2548 | 100 | 100 | 100 | 100 | 100 |
| NS5B | 7979 | A | G | 2547 | N | D | 2550 | 11.8 | — | — | — | — |
| NS5B | 8033 | A | G | 2565 | N | D | 2568 | 5.9 | — | — | — | — |
| NS5B | 8131 | A | T | 2597 | K | N | 2600 | 95.8 | 99.7 | 99.8 | 100 | 100 |
| NS5B | 8134 | A | C | 2598 | K | N | 2601 | 8.6 | — | — | — | — |
| NS5B | 8365 | T | G | 2675 | D | E | 2678 | 100 | 100 | 100 | 100 | 100 |
| NS5B | 8718 | T | C | 2793 | V | A | 2796 | 6.1 | 88.4 | 100 | 100 | 100 |
| NS5B | 8730 | A | G | 2797 | K | R | 2800 | 100 | 100 | 100 | 100 | 100 |
| NS5B | 8756 | G | A | 2806 | E | T | 2809 | 100 | 100 | 100 | 100 | 100 |
| NS5B | 8757 | A | C | | | | | 100 | 100 | 100 | 100 | 100 |
| NS5B | 9087 | C | T | 2916 | A | V | 2919 | 100 | 100 | 100 | 100 | 100 |
| NS5B | 9132 | A | G | 2931 | Q | R | 2934 | 98.1 | 97.3 | 100 | —^{c} | —^{c} |
| NS5B | 9267 | A | G | 2976 | D | G | 2979 | 100 | 100 | 100 | 100 | 100 |
| NS5B | 9273 | A | T | 2978 | Y | F | 2981 | 100 | 100 | 100 | 100 | 100 |
| NS5B | 9281 | A | G | 2981 | M | V | 2984 | 100 | 100 | 100 | 100 | 100 |
| NS5B | 9284 | T | C | 2982 | S | P | 2985 | 26.0 | 90.1 | 100 | 100 | 100 |
| NS5B | 9289 | T | G | 2983 | H | Q | 2986 | 6.3 | 7.0 | — | — | — |
| NS5B | 9312 | T | G | 2991 | L | R | 2994 | 100 | 100 | 100 | 100 | 100 |
| NS5B | 9315 | G | A | 2992 | C | Y | 2995 | 99.3 | 99.1 | 100 | 100 | 100 |

Note:
Only mutations with a frequency of >5% of the viral population in NGS analysis are shown. Shaded background indicates frequencies of engineered mutations. SNP, Single nucleotide polymorphism. NT, nucleotide. AA, amino acid. NT pos and AA pos, 5 nucleotide and amino acid positions, respectively.
^{a}Nucleotide G at position 38 in 5′UTR was changed to A.
^{b}HCV infectivity titers at indicated passage and day (parentheses) are shown as log₁₀FFU/mL.
^{c}These recombinant viruses had A at position 9132 instead of G, and this nt was maintained.

TABLE 2
NGS analysis of recombinant ED43 viruses harboring
RASs recovered from the 2^{nd} passage culture

| | | | | | | | | SNP frequencies (%) | | |
|---|---|---|---|---|---|---|---|---|---|---|
| | Polyprotein | | | | | | P# | NS3^{a} | | |
| HCV | NT | | | AA | | | AA | Y56H, | | A156M, |
| gene | pos | NT change | | pos | AA change | | pos | D168A | A156M | I2841V |
| NS3 | 3584 | T | C | 1082 | Y | H | 56 | — | — | — |
| NS3 | 3884 | G | A | 1182 | A | M | 156 | — | 99.6 | 99.8 |
| NS3 | 3885 | C | T | | | | | — | 99.6 | 99.7 |
| NS3 | 3921 | A | C | 1194 | D | A | 168 | — | — | — |
| NS3 | 4265 | G | C | 1309 | A | P | 283 | — | — | — |
| NS3 | 5268 | A | G | 1643 | K | R | 617 | — | — | — |
| NS4B | 5645 | A | G | 1769 | I | V | 58 | — | — | — |
| NS5A | 6338 | C | G | 2000 | L | V | 28 | — | — | — |
| NS5A | 6338 | C | A | 2000 | L | M | 28 | — | — | — |
| NS5A | 6339 | T | C | 2000 | L | P | 28 | — | — | — |
| NS5A | 6344 | C | T | 2002 | L | F | 30 | — | — | — |
| NS5A | 6344 | CTC | del^{c} | 2002 | L | del^{c} | 30 | — | — | — |
| NS5A | 6345 | T | C | 2002 | L | P | 30 | — | — | — |
| NS5A | 6345 | T | A | 2002 | L | H | 30 | — | — | — |
| NS5A | 6347 | A | G | 2003 | M | V | 31 | — | — | — |
| NS5A | 6480 | C | T | 2047 | T | I | 75 | — | — | — |
| NS5A | 6510 | A | G | 2057 | H | R | 85 | — | — | — |
| NS5A | 6533 | T | C | 2065 | Y | H | 93 | — | — | — |
| NS5A | 6637 | C | A | 2099 | F | L | 127 | — | — | — |
| NS5A | 6737 | C | T | 2133 | H | Y | 161 | — | — | — |
| NS5A | 7266 | A | T | 2309 | H | L | 337 | 97.2 | — | — |
| NS5A | 7578 | G | A | 2413 | G | D | 441 | — | 5.5 | — |
| NS5B | 8406 | A | G | 2689 | D | G | 272 | — | 5.3 | — |
| NS5B | 8435 | A | T^{d} | 2699 | S | C | 282 | — | — | — |
| NS5B | 8436 | G | C | 2699 | S | T | 282 | — | — | — |
| NS5B | 8861 | A | G | 2841 | I | V | 424 | — | 57.2 | 99.9 |

| | SNP frequencies (%) | | | | | | | |
|---|---|---|---|---|---|---|---|---|
| | NS5A^{a} | | | | | | | |
| | | | | L28M, | | | NS5B^{a} | |
| HCV | | L30P, | L30H, | L30F, | | L30Δ^{b}, | | S282T, |
| gene | L28V | Y93H | M31V | M31V | L30Δ^{b} | T2047I | S282T | A1309P |
| NS3 | — | — | — | — | — | — | — | — |
| NS3 | — | — | — | — | — | — | — | — |
| NS3 | — | — | — | — | — | — | — | — |
| NS3 | — | — | — | — | — | — | — | — |
| NS3 | — | — | — | — | — | — | — | 99.8 |
| NS3 | — | — | — | — | 13.9 | — | — | — |
| NS4B | — | — | — | — | 27.5 | — | — | — |
| NS5A | 99.6 | — | — | — | — | — | — | — |
| NS5A | — | — | — | 99.9 | — | — | — | — |
| NS5A | — | — | — | — | 7.2 | — | — | — |
| NS5A | — | — | — | 99.8 | — | — | — | — |
| NS5A | — | — | — | — | 97.0 | 97.0 | — | — |
| NS5A | — | 99.5 | — | — | — | — | — | — |
| NS5A | — | — | 99.6 | — | — | — | — | — |
| NS5A | — | — | 99.8 | 99.9 | — | — | — | — |
| NS5A | — | — | — | — | 7.1 | 99.8 | — | — |
| NS5A | — | — | — | — | — | 5.6 | — | — |
| NS5A | — | 99.5 | — | — | — | — | — | — |
| NS5A | — | — | — | — | 8.6 | — | — | — |
| NS5A | — | — | — | — | 23.7 | — | — | — |
| NS5A | — | — | — | — | — | — | — | — |
| NS5A | — | — | — | — | — | — | — | — |
| NS5B | — | — | — | — | — | — | — | — |
| NS5B | — | — | — | — | — | — | 16.9^{d} | — |
| NS5B | — | — | — | — | — | — | 97.4 | 99.6 |
| NS5B | — | — | — | — | — | — | — | — |

Note:
All recombinant viruses harboring RASs were generated using the ED43-31m_opt backbone as shown in FIGS. 2, 3 and 4.
Shaded background indicates frequencies of mutations that were introduced into this recombinant. Only non-synonymous (coding) mutations that represented >5% of the viral population, are shown. SNP, Single nucleotide polymorphism. P#, protein specific numbers. NT, nucleotide. AA, amino acid. NT pos and AA pos, nucleotide and amino acid positions, respectively.
^{a}Target proteins in which the recombinants harboring RASs were numbered.
^{b}This recombinant had a deletion at NS5A position 30.
^{c}Three nucleotides were deleted, resulting in an amino acid deletion.
^{d}The linkage analysis showed that this nucleotide was combined with C at position 8436, resulting in the reversion of S282T with a frequency of 16.9%. Therefore, the actual frequency of S282T was ~80%.

TABLE 3
Sanger sequencing analysis of recovered ED43(C5A) viruses.

| | | HCV gene | NS2 | NS3 | NS3 | NS3 | NS4A | NS4B | NS4B | NS4B | NS4B | NS5A |
|---|---|---|---|---|---|---|---|---|---|---|---|---|
| | | ED43 nucleotide position | 2819 | 4211 | 4733 | 5054 | 5354 | 5697 | 5804 | 5933 | 5949 | 7578 |
| | | Original nucleotide | A | A | A | T | G | C | A | A | G | A |
| Recombinant | Second passage | Synonymous mutation | | | | | | | | | | |
| virus | titer (day)^{a} | | | | | | | | | | | |
| ED43(C5A)- | 4.0 (13) | T547C, T4303T/C, | G/a | A/G | . | . | T | T | G | G | G/A | . |
| 2m | | T5365A, T7495T/C | | | | | | | | | | |
| | | T7255C, | | | | | | | | | | |
| ED43(C5A)- | 4.1 (13) | None | G | G | . | T/c/a | T | T | G | G | A | . |
| 7m | | | | | | | | | | | | |
| ED43(C5A)- | 4.2 (11) | None | G | G | G | . | T | T | G | G | A | G |
| 9m | | | | | | | | | | | | |
| | | ED43 amino acid position | 827 | 1291 | 1465 | 1572 | 1672 | 1786 | 1822 | 1865 | 1870 | 2413 |
| | | H77 amino acid position | 827 | 1291 | 1465 | 1572 | 1672 | 1786 | 1822 | 1865 | 1870 | 2416 |
| | | Amino acid change | T-A | I-V | S-G | F-L/I^{b} | A-S | A-V | T-A | T-A | S-N | D-G |

Note:
Nucleotide changes resulting in amino acid substitutions are shown. Letters with shaded background indicate the engineered mutations. Acquired mutations are indicated with only capital letters (complete nucleotide changes), or capital/capital letters (50/50 quasispecies), or capital/lower letters (major/minor change). “Dots” indicate identical nucleotides with the original sequence. “None”; no synonymous mutations were found in recovered viruses. See also FIG. 1A.
^{a}The infectivity titers are shown as log₁₀FFU/mL.
^{b}Nucleotide change T to C and T to A results in amino acid substitution F to L and F to I, respectively.

### REFERENCES

Altschul et al. (1997). Gapped BLAST and PSI-BLAST: a new generation of protein database search programs. Nucleic acids research 25, 3389-3402; 10.1093/nar/25.17.3389.
Altschul et al. (2005). Protein database searches using compositionally adjusted substitution matrices. The FEBS journal 272, 5101-5109; 10.1111/j.1742-4658.2005.04945.×.
Fahnøe et al. (2019). Full-Length Open Reading Frame Amplification of Hepatitis C Virus. Methods Mol Biol 1911:85-91.
Gottwein et al. (2010). Novel infectious cDNA clones of hepatitis C virus genotype 3a (strain S52) and 4a (strain ED43): genetic analyses and in vivo pathogenesis studies. J Virol 84(10): 5277-93.
Gottwein et al. (2011). Differential efficacy of protease inhibitors against HCV genotypes 2a, 3a, 5a, and 6a NS3/4A protease recombinant viruses. Gastroenterology 141(3):1067-79.
Gottwein et al. (2018). Efficacy of NS5A Inhibitors Against Hepatitis C Virus Genotypes 1-7 and Escape Variants. Gastroenterology 154(5):1435-48.
Jensen et al. (2019). Evolutionary Pathways to Persistence of Highly Fit and Resistant Hepatitis C Virus Protease Inhibitor Escape Variants. Hepatology 70(3):771-87.
Li et al. (2012a). Highly efficient full-length hepatitis C virus genotype 1 (strain TN) infectious culture system. Proc Natl Acad Sci USA 109(48):19757-62.
Li et al. (2012b). Robust full-length hepatitis C virus genotype 2a and 2b infectious cultures using mutations identified by a systematic approach applicable to patient strains. Proc Natl Acad Sci USA 109(18):E1101-E1110.
Li et al. (2014). Differential sensitivity of 5′UTR-NS5A recombinants of hepatitis C virus genotypes 1-6 to protease and NS5A inhibitors. Gastroenterology 146(3):812-21.
Li et al. (2015). Efficient infectious cell culture systems of the hepatitis C virus (HCV) prototype strains HCV-1 and H77. J Virol 89(1):811-23.
Mathiesen et al. (2015). Adaptive Mutations Enhance Assembly and Cell-to-Cell Transmission of a High-Titer Hepatitis C Virus Genotype 5a Core-NS2 JFH1-Based Recombinant. J Virol 89(15):7758-75.
Pham et al. (2018). HCV Genotype 6a Escape From and Resistance to Velpatasvir, Pibrentasvir, and Sofosbuvir in Robust Infectious Cell Culture Models. Gastroenterology 154(8):2194-208.
Pham et al. (2019) HCV genotype 1-6 NS3 residue 80 substitutions impact protease inhibitor activity and promote viral escape. J Hepatol 70(3):388-97.
Ramirez et al. (2014). Highly efficient infectious cell culture of three hepatitis C virus genotype 2b strains and sensitivity to lead protease, nonstructural protein 5A, and polymerase inhibitors. Hepatology 59(2):395-407.
Ramirez et al. (2016). Robust HCV Genotype 3a Infectious Cell Culture System Permits Identification of Escape Variants With Resistance to Sofosbuvir. Gastroenterology 151(5):973-85.
Ramirez et al. (2018). Current status and future development of infectious cell-culture models for the major genotypes of hepatitis C virus: Essential tools in testing of antivirals and emerging vaccine strategies. Antiviral Res 158:264-87.
Wakita et al. (2005). Production of infectious hepatitis C virus in tissue culture from a cloned viral genome. Nat Med 11(7):791-6.
Watanabe et al. (2020). Establishment of infectious genotype 4 cell culture-derived hepatitis C virus. J Gen Virol 101(2):188-97.

1. An isolated nucleic acid molecule which encodes human hepatitis C virus (HCV) or a HCV replication system of genotype 4a, strain ED43, wherein the said molecule comprises a nucleic acid sequence encoding an amino acid sequence with a sequence identity of at least 99% to SEQ ID NO: 1 or a fragment hereof encoding the said HCV replication system corresponding to NS3-NS5B between amino acids 1027-3008 of SEQ ID NO: 1 or SEQ ID NO: 2, or an amino acid sequence with a sequence identity of at least 99% to the amino acids 1027-3008 of SEQ ID NO: 1 or SEQ ID NO: 2, and wherein the amino acid sequence or the fragment hereof
comprises the following adaptive mutations: 11291V, S1465G, A1672S, A1786V, T1822A, T1865A, S1870N, D2413G, D2545E, D2675E, K2797R, E2806T, A2916V, D2976G, Y2978F, M2981V, L2991R and C2992Y according to SEQ ID NO: 1;
optionally, further comprises the following adaptive mutation T827A according to SEQ ID NO: 1; and
one of the following groups of additional adaptive mutations:
a) Q2931R according to SEQ ID NO: 1;
b) V271G, C458R, Y848C, L1466M, F1572L, G1909A, A2257T, T2329A, K2597N, V2793A, Q2931R and S2982P according to SEQ ID NO: 1; or
c) V271G, C458R, Y848C, L1466M, F1572L, G1909A, A1973T, A2257T, T2329A, K2597N, V2793A, and S2982P according to SEQ ID NO: 1.

2-25. (canceled)

26. The isolated nucleic acid molecule according to claim 1, wherein the nucleic acid molecule further comprises an adaptive mutation in the 5′UTR region, said adaptive mutation being G38A according to SEQ ID NO: 6.

27. The isolated nucleic acid molecule according to claim 1, wherein said molecule comprises said additional adaptive mutations of group c).

28. The isolated nucleic acid molecule according to claim 1, wherein said nucleic acid molecule is strain ED43cc (SEQ ID NO: 7), strain ED43-31m opt (SEQ ID NO: 8), strain ED43-31m (SEQ ID NO: 9) or strain ED43-20m (SEQ ID NO: 10).

29. The isolated nucleic acid molecule according to claim 1, wherein said nucleic acid molecule encodes an amino acid sequence according to SEQ ID NO: 2 and further comprises an adaptive mutation being G38A according to SEQ ID NO: 6.

30. The isolated nucleic acid molecule according to claim 1, wherein said nucleic acid molecule encodes the fragment encoding the said HCV replication system corresponding to NS2-NS5B between amino acids 810-3008 of SEQ ID NO: 1 or SEQ ID NO: 2, or an amino acid sequence with a sequence identity of at least 99% to the amino acids 810-3008 of SEQ ID NO: 1 or SEQ ID NO: 2.

31. The isolated nucleic acid molecule according to claim 1, wherein said nucleic acid molecule further comprises the 5′UTR region between nucleic acids 1-340 of SEQ ID NO: 6 or SEQ ID NO: 7, or a nucleic acid sequence with a sequence identity of at least 99% to nucleic acids 1-340 of SEQ ID NO: 6 or SEQ ID NO: 7.

32. The isolated nucleic acid molecule according to claim 1, wherein said nucleic acid molecule further comprises the 3′UTR region between nucleic acids 9368-9577 of SEQ ID NO: 6 or SEQ ID NO: 7, or a nucleic acid sequence with a sequence identity of at least 99% to nucleic acids 9368-9577 of SEQ ID NO: 6 or SEQ ID NO: 7.

33. The isolated nucleic acid molecule according to claim 1, wherein said molecule is capable of generating an HCV infectivity titer of 2 log₁₀ FFU/ml (focus forming units)/ml or above following transfection and/or subsequent viral passage.

34. A method for producing a hepatitis C virus particle or a HCV replication system, comprising culturing a cell comprising the nucleic acid molecule of claim 1 and producing hepatitis C virus particles or the HCV replication system therefrom.

35. The method according to claim 34, wherein the cell is Huh7.5.

36. An immunogenic composition comprising a hepatitis C virus particle produced by the method of claim 34 or an immunogenic part thereof.

37. A method for producing an immunogenic response in a subject comprising administering a hepatitis C virus particle produced by the method of claim 34 to a subject.

38. A method for producing a cell, which replicates human hepatitis C virus and produces a virus particle comprising introducing a nucleic acid molecule as described in claim 1 into a cell.

39. The method according to claim 38, further comprising culturing the cell to produce the human hepatitis C virus particle.

40. The method according to claim 38, further comprising infecting other cells with the produced human hepatitis C virus particle.

41. A method for producing a hepatitis C virus replication system, comprising culturing a cell according to claim 38 to allow the cell to replicate the virus genome.

42. A method for screening an anti-hepatitis C virus substance, comprising
a) culturing a cell comprising: a nucleic acid molecule which encodes human hepatitis C virus (HCV) or a HCV replication system of genotype 4a, strain ED43, wherein the said molecule comprises a nucleic acid sequence encoding an amino acid sequence with a sequence identity of at least 99% to SEQ ID NO: 1 or a fragment hereof encoding the said HCV replication system corresponding to NS3-NS5B between amino acids 1027-3008 of SEQ ID NO: 1 or SEQ ID NO: 2, or an amino acid sequence with a sequence identity of at least 99% to the amino acids 1027-3008 of SEQ ID NO: 1 or SEQ ID NO: 2, and wherein the amino acid sequence or the fragment hereof
comprises the following adaptive mutations: 11291V, S1465G, A1672S, A1786V, T1822A, T1865A, S1870N, D2413G, D2545E, D2675E, K2797R, E2806T, A2916V, D2976G, Y2978F, M2981V, L2991R and C2992Y according to SEQ ID NO: 1;
optionally, further comprises the following adaptive mutation T827A according to SEQ ID NO: 1; and
one of the following groups of additional adaptive mutations:
d) Q2931R according to SEQ ID NO: 1;
e) V271G, C458R, Y848C, L1466M, F1572L, G1909A, A2257T, T2329A, K2597N, V2793A, Q2931R and S2982P according to SEQ ID NO: 1; or V271G, C458R, Y848C, L1466M, F1572L, G1909A, A1973T, A2257T, T2329A, K2597N, V2793A, and S2982P according to SEQ ID NO: 1
b) detecting the replicating RNA or the virus particles in the resulting culture.
